(19) United States
(12) Patent Application Publication
(10) Pub. No.: US 2015/0072349 A1
(43) Pub. Date: Mar. 12, 2015

(54) Cancer Biomarkers and Methods of Use

(71) Applicant: University Health Network, Toronto (CA)

(72) Inventors: Eleftherios P. Diamandis, Toronto (CA); Ioannis Prassas, Toronto (CA); Shalini Makawita, Toronto (CA); Caitlin Chrystoja, Toronto (CA); Hari M. Kosanam, Maple (CA)

(21) Appl. No.: 14/385,449

(22) PCT Filed: Mar. 15, 2013
(86) PCT No.: PCT/CA2013/000248

§ 371 Date: Sep. 23, 2014

Related U.S. Application Data
(60) Provisional application No. 61/611,955, filed on Mar. 16, 2012.

(51) Int. Cl. G01N 33/574

(52) U.S. Cl. CPC G01N 33/57484; G01N 2333/705
USPC 435/6.12; 435/7.94

(57) ABSTRACT

A method of evaluating a probability a subject has a cancer, diagnosing a cancer and/or monitoring cancer progression comprising: a. measuring an amount of a biomarker selected from the group consisting of CUZD1 and/or LAMC2 and/or the group CUZD1, LAMC2, AQP8, CELA2B, CELA3B, CTRB1, CTRB2, GCG, IAPP, INS, KLK1, PNLIPRP1, PNLIPRP2, PPY, PRSS3, REG3G, SLC30A8, KLK3, NPY, PSCA, RLN1, SLC45A3, DSP, GP73, DSG2, CEACAM7, CLCA1, GPA33, LEFTY1, ZG16, IRX5, LAMP3, MFAP4, SCGB1A1, SFTPC, TMEM100, NPY, PSCA, RLN1 and/or SLC45A3 in a test sample from a subject with cancer; wherein the cancer is pancreas cancer if CUZD1, LAMC2, AQP8, CELA2B, CELA3B, CTRB1, CTRB2, GCG, LAPP, INS, KLK1, PNLIPRP1, PNLIPRP2, PPY, PRSS3, REG3G, SLC30A8, DSP, GP73 and/or DSG2 is selected; the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; or the cancer is prostate cancer if NPY, PSCA, RLN1 and/or SLC45A3 is selected; b. comparing the measured amount to a control and detecting an increase in the amount of the biomarker compared to control; and c. identifying the subject as having or having an increased probability of having the cancer when an increase in the biomarker compared to control is detected.

### RELATED APPLICATIONS

This application is a PCT application which claims priority from U.S. provisional 61/611,955 filed March 16, which is herein incorporated by reference in its entirety.

### FIELD

The disclosure relates to cancer biomarkers and more particularly to tissue specific serum cancer biomarkers and methods and uses thereof.

### INTRODUCTION

Serological biomarkers represent a non-invasive and cost-effective means to aid in clinical management of cancer patients, particularly in areas of disease detection, prognosis, monitoring and therapeutic stratification. For a serological biomarker to be useful for early detection, its presence in serum must be relatively low in healthy individuals and those with benign disease. The marker must be produced by the tumor or its microenvironment and enter circulation, giving rise to increased serum levels. Mechanisms that facilitate entry to circulation include secretion or shedding, angiogenesis, invasion, and destruction of tissue architecture [1]. The biomarker should preferably be tissue specific, such that a change in serum level can be directly attributed to disease (e.g., cancer) of that tissue [2]. The currently most widely-used serological biomarkers include carcinoembryonic antigen (CEA) and carbohydrate antigen 19.9 (CA19.9) for gastrointestinal cancer [3-5], CEA, CYFRA 21-1 (cytokeratin 19 fragment), neuron-specific enolase (NSE), tissue polypeptide antigen (TPA), progastrin-releasing peptide (pro-GRP), and SCC antigen for lung cancer [6], CA 125 for ovarian cancer [2], and prostate-specific antigen (PSA, also known as KLK3) in prostate cancer [7]. These current serological biomarkers lack the appropriate sensitivity and specificity to be suitable for early cancer detection.

An example of Serum PSA is commonly used for prostate cancer screening in men over 50, but its usage remains controversial due to serum elevation in benign disease as well as prostate cancer [8]. Nevertheless, PSA represents one of the most useful serological markers currently available. PSA is strongly expressed in only the prostate tissue of healthy men, with low levels in serum established by normal diffusion through various anatomical barriers. These anatomical barriers are disrupted upon development of prostate cancer, allowing increased amounts of PSA to enter circulation [1].

### SUMMARY

In an aspect, the disclosure includes a method of evaluating a probability a subject has a cancer and/or diagnosing the subject with cancer, the method comprising:
a. measuring an amount of a biomarker selected from the group consisting of CUZD1 and/or LAMC2 and/or the group CUZD1, LAMC2, AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, GP73, DSG2, CEACAM7, CLCA1, GPA33, LEFTY1, ZG16, IRX5, LAMP3, MFAP4, SCGB1A1, SFTPC, TMEM100, NPY, PSCA, RLN1 and/or SLC45A3 in a test sample from a subject with cancer; wherein the cancer is pancreas cancer if CUZD1, LAMC2, AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, GP73 and/or DSG2 is selected; the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; or the cancer is prostate cancer if NPY, PSCA, RLN1 and/or SLC45A3 is selected;
b. comparing the measured amount to a control and detecting an increase in the amount of the biomarker compared to control; and
c. identifying the subject as having or having an increased probability of having the cancer when an increase in the biomarker compared to control is detected.
   In another aspect, the disclosure includes a method of monitoring cancer progression, the method comprising:

In another aspect, the disclosure includes a method of monitoring cancer progression, the method comprising:
a. obtaining a test sample from the subject,
b. measuring an amount of biomarker according to the method described herein the test sample;
c. comparing the measured amount of biomarker in the test sample to the amount of biomarker in a base-line sample for the subject and/or a control; and
d. identifying a difference in the amount of the biomarker between the test sample and the base-line sample for the subject and/or the control;
wherein an increase in biomarker amount in the test sample compared to the base-line sample and/or the control is indicative of progression and a decrease in biomarker amount is indicative of lack of progression.

In an embodiment, the biomarkers comprise CUZD1 and/or LAMC2.

In yet another aspect, the disclosure includes a method of monitoring pancreatic cancer progression, the method comprising:
a. obtaining a test sample from the subject,
b. measuring an amount of CUZD1 and/or LAMC2 in the test sample;
c. comparing the amount of CUZD1 and/or LAMC2 in the test sample to amount of CUZD1 and/or LAMC2 in a base-line sample for the subject and/or control; and
d. identifying a difference in the amount of the CUZD1 and/or LAMC2 between the test sample and the base-line sample and/or control;
wherein an increase in CUZD1 and/or LAMC2 in the test sample compared to the base-line sample is indicative of progression and a decrease in CUZD1 and/or LAMC2 is indicative of lack of progression.

In a further aspect, the disclosure includes a method of validating a candidate biomarker as a cancer biomarker comprising:
a. selecting a candidate biomarker from the group consisting of AQP8, CELA2B, CELA3B, CTRB1, CTRB2, CUZD1, GCG, IAPP, INS, LAMC2, KLK1, PNLIPRP1, PNLIPRP2, PPY, PRSS3, REG3G, SLC30A8, DSP, GP73, DSG2, CEACAM7, CLCA1, GPA33, LEFTY1, ZG16, IRX5, LAMP3, MFAP4, SCGB1A1, SFTPC, TMEM100, NPY, PSCA, RLN1 and/or SLC45A3 in a test sample from a subject with cancer, wherein the cancer is pancreas cancer if AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, and/or GP73 is selected; the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; or the cancer is prostate cancer if NPY, PSCA, RLN1 and/or SLC45A3 is selected;
b. measuring an amount of the selected candidate biomarker according to the method described herein in a plurality of samples from a plurality of subjects with cancer;
c. comparing the measured amount of the selected candidate biomarker in the plurality of test samples to a control;
d. identifying an increase in the amount of the selected candidate biomarker in the plurality of test samples as compared to the control; and
e. identifying a statistically significant increase in the amount of the selected candidate biomarker in the plurality of test samples as compared to the control;
   wherein a statistically significant increased amount of the selected biomarker in the plurality of samples compared to the control is indicative the selected candidate biomarker is a cancer biomarker for the corresponding cancer.

In an embodiment the test sample is a biological fluid.

In another embodiment the biological fluid is blood or a fraction thereof selected from serum and plasma.

In an embodiment the biomarkers is selected from CEACAM7, CLCA1, GPA33, LEFTY1 and/or ZG16.

In an embodiment the biomarker is selected from IRX5, LAMP3, MFAP4, SCGB1A1, SFTPC, and/or TMEM100.

In a further embodiment the biomarker is selected from AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2.

In yet another embodiment the biomarker is selected from NPY, PSCA, RLN1 and SLC45A3.

In an embodiment the control is a cut-off for associated with a specificity and sensitivity and the specificity is selected to be at least 65%, at least 70%, at least 75%, at least 80%, at least 85% or at least 90%.

In an embodiment the sensitivity is selected to be at least 65%, at least 70%, at least 75%, at least 80%, at least 85% or at least 90%.

In another embodiment the amount of CUZD1 indicative the subject has pancreatic cancer or an increased probability of pancreatic cancer is greater than about 2 ng/ml, 2.2 ng/ml, 2.4 ng/ml, 2.6 ng/ml, 2.8 ng/ml, 3 ng/ml, 3.1 ng/ml, 3.2 ng/ml, 3.4 ng/ml, 3.6 ng/ml, 3.8 ng/ml, 4 ng/ml, 4.2 ng/ml, 4.4 ng/ml, 4.6 ng/ml, 4.8 ng/ml, 5 ng/ml.

In an embodiment the amount of LAMC2 indicative the subject has pancreatic cancer or an increased probability of pancreatic cancer is greater than about 100 ng/ml, 120 ng/ml, 140 ng/ml. 160 ng/ml, 170 ng/ml, 180 ng/ml, 200 ng/ml, 220 ng/ml, 240 ng/ml, 260 ng/ml, 280 ng/ml, 300 ng/ml, 320 ng/ml, 340 ng/ml, 360 ng/ml, 380 ng/ml or 400 ng/ml.

In an embodiment, the method further compress measuring the amount of an additional biomarker in the sample.

In a further embodiment the additional biomarker is selected from CA19.9 CEA, CYFRA-21-1 NSE TPA, proGRP, SCC, CA125 and PSA.

In an embodiment the additional biomarker is CA19.9

In an embodiment the biomarker is CUZD1, LAMC2 and/or DSG2 and the additional biomarker is CA19.9.

In another embodiment the measuring comprises an antibody based immunoassay.

In an embodiment the immunoassay is an ELISA.

In an aspect, this disclosure includes the use of a biomarker selected from the group consisting of CUZD1 and/or LAMC2 and/or the group consisting of CEACAM7, CLCA1, GPA33, LEFTY1, ZG16, IRX5, LAMP3, MFAP4, SCGB1A1, SFTPC, TMEM100, AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3, DSP, LAMC2, GP73 and/or DSG2 for evaluating if a subject has cancer according to the method described herein.

In another aspect, the disclosure includes a method of validating a candidate biomarker as a soluble tissue specific cancer biomarker comprising:
a. selecting a candidate biomarker according to the method described herein;
b. measuring an amount of the selected candidate biomarker in a plurality of biological fluid test samples from a plurality of subjects afflicted by the cancer for the candidate marker and comparing to a control;
c. identifying an increase in the amount of the selected biomarker in the plurality of test samples as compared to the control; and;
d. identifying a statistically significant increase in the amount of the selected candidate biomarker in the plurality of biological fluid test samples as compared to the control;
   wherein a statistically significant increased amount of the selected biomarker in the plurality of biological fluid test samples compared to the control is indicative the selected candidate biomarker is a soluble cancer biomarker for the corresponding cancer.

In an embodiment the biological fluid is selected from ascites, seminal plasma, peritoneal fluid, pancreatic juice and/or saliva.

In another embodiment 2, 3, 4, 5, 6, 7 or more biomarkers are measured.

In a further embodiment the biomarkers comprise CUZD1, LAMC2 and CA19.9.

In an aspect, the disclosure includes a kit comprising:
a. a biomarker specific reagent for a biomarker of the disclosure and optionally an additional biomarker; and
b. optionally one or more of
   i. a kit standard;
   ii. instructions for use and a vial housing the biomarker specific reagent and/or kit standard;
   iii. reagents for qRT-PCR, including buffers, reverse transcription and amplification primers for the target genes and endogenous control genes, and control RNA from normal oral tissue;
   iv. reagents for digital molecular barcoding technology, including for example buffers, hybridization solution, and/or one or more labeled probes;
   v. collection tubes and/or assay plates for conducting one or more assays; and
   vi. a sample collection vessel for example a vacutainer tube or other sterile tube for biological fluid.

In an embodiment two or more antibodies, optionally coupled to a solid surface.

In another embodiment the two or more antibodies comprise an antibody specific for CUDZ1 and an antibody specific for CA19.9.

In an embodiment the kit for use in the method described herein.

In an embodiment, the biomarker is CUZD1.

In an embodiment, the biomarker is LAMC2.

In an embodiment, the biomarker is selected from DSP and GP73

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1. Schematic outline of tissue-specific biomarker identification. Protein identification in seven publicly available gene and protein databases, grouped by the type of data each database is based on, followed by filtering criteria and integration of proteomic datasets to identify and prioritize candidates is outlined. ESTs, expressed sequence tags; TiGER, Tissue-specific and Gene Expression and Regulation; IHC, immunohistochemistry; HPA, Human Protein Atlas.

FIG. 2. Identification of tissue-specific proteins by each database. Venn diagrams depicting which database had initially identified the tissue-specific proteins that passed the filtering criteria (identified in ≧2 databases, designated as secreted or shed, and expression profiles verified in silico). Overlap of tissue-specific proteins identified in databases based off ESTs (a), microarray (b), and three databases that identified the most tissue-specific proteins (c) is also depicted. For details see text.

FIG. 3. Initial validation of CUZD1 and CA19.9 (for comparison) was performed using 20 benign, pancreatic cyst serum samples and 20 pancreatic cancer samples of mixed stages (no healthy individuals included). Receiver operating characteristic (ROC) curve for CA19.9 (A) and CUZD1 (B). At a cutoff of 37 IU/mL, CA19.9 showed 70% specificity and 80% sensitivity (identified six false positives, shown in Table 9 in squares, four false negatives, shown in circles). At a cutoff of 3.1 ng/mL, CUZD1 showed 85% specificity and 85% sensitivity (identified three false positives, shown in Table 9 in squares, and three false negatives, shown in circles). CUZD1 had a similar area under the curve (AUC) value to CA19.9 (Table 10, FIG. 3). Only two of the six samples which CA19.9 identified as false positives were also identified as false positives by CUZD1. None of the samples which CA19.9 identified as false negatives were identified as false negatives by CUZD1. Based on these data, CUZD1 represents a marker with increased sensitivity and specificity than CA19.9. Combination of both CA 19.9 and CUZD1 results in 100% sensitivity but specificity drops slightly from 70% with CA 19.9 alone, to 65%.

FIG. 4. Extended validation of CA19.9 (for comparison) and CUZD1 using 50 normal, 50 benign (e.g. pancreatitis, pancreatic cyst) and 50 pancreatic cancer samples of mixed stages. Scatter Plot: CUZD1 and CA19-9. In this larger dataset, CUZD1 out-performed CA19-9 in discriminating between benign and cancer patients. When the results of CA19-9 were examined found that 14 out of the 50 cancer patients were negative for CA19-9 (less than 37 IU/L). However, among these, 8 were positive for CUZD-1 (at a cutoff of 3.1 ng/mL). Notably, the patient in the benign group with high levels of CUZD-1 (˜60 ng/ml) is the same patient with very high levels of CA19-9 (˜3500 U/ml).

FIG. 5. ROC Curve Analysis of CUZD1 and CA19.9 in the extended dataset (50 normal, 50 benign, 50 pancreatic cancer samples). 5A. Normal vs Cancer; CA19-9 and CUZD-1 showed similar efficacies in discriminating between normal and cancer patients 5B. Benign vs Cancer; CUZD1 out-performed CA19-9 in discriminating between benign and cancer patients. 5C: Benign vs PDAC; the combination of CUZD1 and CA19-9 out-performed both CA19-9 and CUZD1 alone in discriminating between benign and cancer patients. Significant complementarity of CUZD1 with CA 19-9 were captured (CUZD1-cutoff used: 4.6 ng/ml).

FIG. 6. Scatter Plot Analysis of LAMC2, DSG2 and CA19-9 using 50 normal, 50 benign and 50 pancreatic cancer samples of mixed stages.

FIG. 7. ROC Curve Analysis of LAMC2, DSG2 and CA19-9 in the extended dataset (50 normal, 50 benign, 50 pancreatic cancer samples). 7A. Normal vs Cancer; LAMC2 out-performed CA19-9 in discriminating between normal and cancer patients. DSG2 has a similar potency to CA19-9 in discriminating between normal and cancer patients 7B. Benign vs Cancer; CA19-9 out-performed both LAMC2 and DSG2 in discriminating between benign and cancer patients.

FIG. 8. Scatter Plot Analysis and ROC Curve Analysis of CUZD1 and CA 19-9 using 50 normal, 50 benign, 50 PDAC-II and 50 PDAC-IV pancreatic cancer samples.

FIG. 9: Scatter Plot Analysis and ROC Curve Analysis of CUZD1 and CA 19-9 using 20 normal, 15 benign, 25 PDAC-II and 25 PDAC-IV pancreatic cancer samples.

FIG. 10: Scatter Plot Analysis of CA19.9, CUZD1, LAMC2 in the training and validation cohorts. 10A. CA19.9 for training cohort. 10B. CA19.9 for validation cohort. 10C. CUZD1 for training cohort. 10D. CUZD1 for validation cohort. 10E. LAMC2 for training cohort. 10F. LAMC2 for validation cohort. Black horizontal lines are medians. PDAC=pancreatic ductal adenocarcinoma.

FIG. 11: ROC Curves. 11A. Diagnostic performances of CA19.9, CUZD1 and LAMC2 for all PDAC patients versus benign patients as individual markers. (i) ROC curves for CA19.9, CUZD1 and LAMC2, for all patients with PDAC versus all benign patients as individual markers in the training cohort. (ii) ROC curves for CA19.9, CUZD1 and LAMC2, for all patients with PDAC versus all benign patients as individual markers in the validation cohort. ROC=receiver operating characteristics. PDAC=pancreatic ductal adenocarcinoma. 11B: Diagnostic performances of CA19.9, CUZD1 and LAMC2, for all PDAC patients versus benign patients as individual markers 11C. Complementarity of CA19.9, CUZD1 and LAMC2 in differentiating all patients with PDAC versus all benign patients A) ROC curves for CA19.9, CA19.9+CUZD1, CA19.9+LAMC2 and CA19.9+CUZD1+LAMC2 multiple markers models for all patients with PDAC versus all benign patients in the training cohort. (B) ROC curves for CA19.9, CA19.9+CUZD1, CA19.9+LAMC2 and CA19.9+CUZD1+LAMC2 multiple markers models for all patients with PDAC versus all benign patients in the validation cohort. ROC=receiver operating characteristics. PDAC=pancreatic ductal adenocarcinoma. 11D. Complementarity of CA19.9, CUZD1 and LAMC2 in differentiating all PDAC patents versus all benign patients 11E. Diagnostic performances of CA19.9, CUZD1 and LAMC2, for Stage IA IB and IIA PDAC patients versus benign patients as individual markers 11F: Complementarity of CA19.9, CUZD1 and LAMC2 in differentiating Stage IA, IB and IIA PDAC patients versus all benign patients. 11G: Diagnostic performances of CA19.9, CUZD1 and LAMC2, for Stage IA, IB, IIA, and IIB PDAC patients versus benign patients as individual markers. 11H: Complementarity of CA19.9, CUZD1 and LAMC2 in differentiating Stage IA, IB, IIA and IIB PDAC patients versus all benign patients.

FIG. 12: Specificity/sensitivity of CA19.9 vs CUZD1 vs LAMC2 and complementarity of CA19.9, CUZD1 and LAMC2 in differentiating Benign vs different stage cancers

FIG. 13: CA19-9 and CUZD1 quadrant plot: CUZD1 can discriminate better between early stage and late stage cancers than CA19.9.

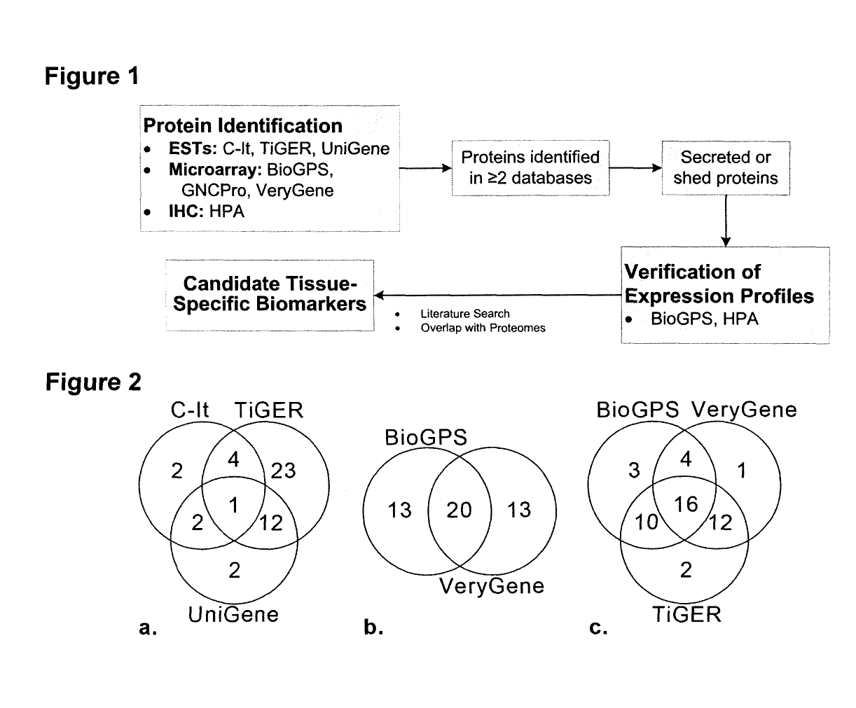

### DETAILED DESCRIPTION

### I. Definitions

Abbreviations used include: CEA, carcinoembryonic antigen; CA19.9, carbohydrate antigen 19.9; CYFRA 21-1, cytokeratin 19 fragment; NSE, neuron-specific enolase; TPA, tissue polypeptide antigen; pro-GRP, progastrin-releasing peptide; PSA, prostate-specific antigen; TiGER, Tissue-specific and Gene Expression and Regulation; ESTs, expressed sequence tags; HPA, Human Protein Atlas; IHC, immunohistochemistry; MeSH, Medical Subject Headings; CLCA4, chloride channel accessory 4; SFPTA2, surfactant protein A2; PNLIP, pancreatic lipase; KLK3, kallikrein-related peptidase 3 The full names of biomarkers are found in the Tables, and the associated sequences as indicated by the provided accession numbers, incorporated herein by reference.

The term “antibody” as used herein is intended to include monoclonal antibodies, polyclonal antibodies, and chimeric antibodies. The antibody may be from recombinant sources and/or produced in transgenic animals.

The term “antibody binding fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies may be monospecific, bispecific, trispecific or of greater multispecificity. Multispecific antibodies may immunospecifically bind to different epitopes of a NADPH oxidase polypeptide and/or or a solid support material. Antibodies may be from any animal origin including birds and mammals (e.g., human, murine, donkey, sheep, rabbit, goat, guinea pig, camel, horse, or chicken).

Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies.

In aspects, the antibody is a purified or isolated antibody. By “purified” or “isolated” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

The term “biomarker” or “biomarker of the disclosure” as used herein means a biomarker listed in Table 4 and/or 11 and/or the subset listed in Tables 5, 6, 7, 8 and/or 11, fragments and naturally occurring variants thereof. The biomarker can be for example used to aid in the evaluation of the presence of a cancer of a specific tissue type. For example, Table 5 lists proteins that are specific to colon tissue and they may represent colon cancer specific biomarkers; Table 6 lists proteins that are specific to lung tissue and they may represent lung cancer specific biomarkers; Table 7 and 11 list proteins that are specific to pancreas tissue and they may represent pancreas cancer specific biomarkers, for example as shown for CUZD1, LAMC2 and DSG2; Table 8 lists proteins that are specific to prostate tissue and they may represent prostate cancer specific biomarkers.

The term “CUZD1” as used herein refers to “CUB and zona pellucid-like domain-containing protein 1” which is also referred to a UO-44. The gene is located on chromosome 10q26.13 and encodes a 607 amino acid transmembrane protein. CZUD1 includes without limitation, all known CUZD1 molecules, including human, naturally occurring variants and those deposited in Genbank, for example, with accession number Q86UP6 and/or NP_{—}071317, and Swiss-Prot ID of Q86UP6, each of which is herein incorporated by reference.

The term “LAMC2” as used herein refers to laminin, gamma C2 and includes without limitation all known LAMC2 molecules, including human, naturally occurring variants and those deposited in publically available databases with different accession numbers, such as HGNC_{—}64931, Entrez Gene_{—}39182,Ensembl_ENSG000000580857,OMIM_{—}1502925,UniProtKB_Q137533 each of which is herein incorporated by reference.

The term “additional biomarker” as used herein means a biomarker not listed in Table 5, 6, 7, 8 or 11 and includes biomarkers used in clinic for example CA19.9 CEA, CYFRA-21-1 NSE TPA, proGRP, SCC, CA125 and PSA. Other additional biomarkers include for example, biomarkers listed in Table 4 as previously studied, for example SFTPA2, SFTPB, SFTPD, CEL, CELA2A, CPA1, CPA2, CPB1, PNLIP, PRSS1, SYCN, ACPP, FOLH1, KLK2 and/or KLK3.

The phrase “biomarker polypeptide”, “polypeptide biomarker” or “polypeptide product of a biomarker” refers to a proteinaceous biomarker gene product for example of a biomarker listed in Table 4 and/or 11.

The phrase “biomarker nucleic acid”, or “nucleic acid product of a biomarker” refers to a polynucleotide biomarker gene product of a biomarker for example a biomarker listed in tables 4 and/or 11.

The term “biomarker specific reagent” as used herein refers to a reagent that is a highly sensitive and specific, for example exhibiting at least 2×, at least 3×, at least 4× at least 5 or at least 10× greater specificity for its cognate antigen compared to another antigen, for quantifying levels of a biomarker expression product, for example a polypeptide biomarker level or a nucleic acid biomarker product and can include antibodies which can for example be used with immunohistochemistry (IHC), ELISA and protein microarray or polynucleotides such as primers and probes which can for example be used with quantitative RT-PCR techniques, to detect the expression level of a biomarker associated with a cancer.

The term “control” as used herein refers to any sample or samples from a subject without cancer or not having the cancer being tested, of a similar type to the test sample which can be used for measuring control biomarker expression levels and/or predetermined value or reference standard which corresponds to and/or is derived from biomarker levels expressed for example as a numerical value (e.g. cut-off) corresponding to the biomarker levels in such a control sample or samples. For example the control can be an average, median, normalized level or cut-off value (e.g. threshold) for a biomarker above or below which a subject can be classified as likely having or not having a cancer.

The cut-off or threshold can for example be a median level or value comprising the median expression level or levels in a population of subjects, e.g. below which are likely not to have cancer and above which are likely to have cancer. For example following a clinical study which can be similar to the study described in Example 2 or Example 8, a cut-off or threshold can be determined to optimize the trade-off between false negative and false positive discoveries, for example by optimizing the area under the ROC curve. The optimized threshold will for example vary with the number of biomarkers being assessed (e.g. CUZD1 vs CUZD1 and CA19.9) The threshold(s) may be set at a desired sensitivity or specificity and/or to correspond to a selected level based on the study sample, for example corresponding to the lowest 90%, 80%, 70%, 60%, 50%, 40%, 30%, 20% or 10% of in a population of subjects. The expression levels compared, can be normalized levels wherein the expression level for example in the test sample is compared to an internal standard and used to calculate a ratio. For example an internal standard is a non-biomarker gene (transcript or protein) that is suitable for comparison (e.g. expected to be expressed at relatively the same level in different samples) that is used to quantify the relative amount of biomarker transcript for comparison purposes. The ratio is then compared to a similar ratio in a control sample and/or a predetermined ratio corresponding to control samples.

As an example, an optimized cutoff for each marker can be obtained by minimizing the total prediction error, using for example the following formula: \sqrt{(1−sensitivity)²+(1−specificity)²}. Cutoffs can be chosen based on the shortest distance of the ROC curve to the top-left corner. Multi-parametric models for combinations of markers can be used to obtain estimated coefficients. The estimated coefficients of the model can be used to construct a combined score for each observation which is then used for the evaluation of the multi-parametric model. Typically, both a training and a validation set of samples is used. Analysis of the results from the training dataset can identify the optimized cut-offs that are subsequently verified in a validation set.

The term “measuring an expression level” as used in reference to a biomarker means the application of a biomarker specific reagent such as a probe, primer or antibody and/or a method to a sample, for example a sample of the subject and/or a control sample, for ascertaining or measuring quantitatively, semi-quantitatively or qualitatively the amount of a biomarker or biomarkers, for example the amount of biomarker polypeptide or mRNA. For example, a level of a biomarker can be determined by a number of methods including for example immunoassays including for example immunohistochemistry, ELISA, Western blot, immunoprecipation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “difference in the level” as used herein in comparison to a control refers to a measurable difference in the level or quantity of a biomarker or biomarkers associated in a test sample, compared to the control that is of sufficient magnitude to allow assessment of predicted outcome, for example a significant difference or a statistically significant difference. The magnitude of the difference is sufficient for example to determine that the subject falls within a class of subjects likely to have disease and/or not have disease. For example, a difference in a level of biomarker level is detected if a ratio of the level in a test sample as compared with a control is greater than 1.5 for example, a ratio of greater than 1.7, 2, 3, 3, 5, 10, 12, 15, 20 or more.

The term “digital molecular barcoding technology” as used herein refers to a digital technology that is based on direct multiplexed measurement of gene expression that utilizes color-coded molecular barcodes, and can include for example Nanostring nCounter™. For example, in such a method each color-coded barcode is attached to a target-specific probe, for example about 50 bases to about 100 bases or any number between 50 and 100 in length that hybridizes to a gene of interest. Two probes are used to hybridize to mRNA transcripts of interest: a reporter probe that carries the color signal and a capture probe that allows the probe-target complex to be immobilized for data collection. Once the probes are hybridized, excess probes are removed and detected. For example, probe-target complexes can be immobilized on a substrate for data collection, for example an nCounter™ Cartridge and analysed for example in a Digital Analyzer such that for example color codes are counted and tabulated for each target molecule.

The term “expression level” as used herein in reference to a biomarker refers to a quantity of biomarker that is detectable or measurable in a sample and/or control. The quantity is for example a quantity of polypeptide, or a quantity of nucleic acid e.g. biomarker transcript. Accordingly, a polypeptide expression level refers to a quantity of biomarker polypeptide that is detectable or measurable in a sample and a nucleic acid expression level refers to a quantity of biomarker nucleic acid that is detectable or measurable in a sample.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, hybridization in 6.0× sodium chloride/sodium citrate (SSC) at about 45° C., followed by a wash of 2.0×SSC at 50° C. may be employed.

The term “kit standard” as used herein means a suitable assay standard useful when determining an expression level of a biomarker associated with a cancer disclosed herein. For example, for kits for determining polypeptide biomarker levels, the kit standard optionally comprises a biomarker polypeptide (or peptide fragment) that can for example be used to prepare a standard curve or act as a positive antibody control. Alternatively, the kit standard is an antibody to a non-biomarker polypeptide such as actin for determining relative biomarker levels. For kits for detecting RNA levels for example by hybridization, the kit standard can comprise an oligonucleotide control, useful for example for internal normalization such as GAPDH for standardizing the amount of RNA in the sample and determining relative biomarker transcript levels. The kit standard can also comprise one or more known oligonucleotides that can be used to detect transcript levels of normalization genes, for example, one or more housekeeping genes, for example, genes with approximate constant expression across samples.

The term “primer” as used herein refers to a polynucleotide, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand.

The term “probe” as used herein refers to a nucleic acid sequence that will hybridize to a nucleic acid target sequence. In one example, the probe hybridizes to a biomarker RNA or a nucleic acid sequence complementary to the biomarker RNA. The length of probe depends for example, on the hybridization conditions and the sequences of the probe and nucleic acid target sequence. The probe can be for example, at least 15, 20, 25, 50, 75, 100, 150, 200, 250, 400, 500 or more nucleotides in length.

A person skilled in the art would recognize that “all or part of” a particular probe or primer can be used as long as the portion is sufficient for example in the case a probe, to specifically hybridize to the intended target and in the case of a primer, sufficient to prime amplification of the intended template.

The term “sample” as used herein refers to any biological fluid, or tissue or fraction thereof (e.g. tissue extract, membrane extract, cytosolic extract, plasma or serum in the case of blood) from a subject that can be assessed for biomarker expression products, polypeptide expression products or nucleic acid expression products, including for example an isolated RNA fraction, optionally mRNA for nucleic acid biomarker determinations and a protein fraction for polypeptide biomarker determinations, and includes for example fresh tissue, frozen cells/tissue and fixed cells/tissue including formalin fixed, paraffin embedded (FFPE) samples. The sample can for example be a test sample which is a patient sample to be tested or a control sample which is a sample (or plurality of samples) with known outcome used for comparison. The biological fluid can for example be a blood fraction such as serum or blood (e.g. in the case of pancreas, colon, lung and prostate). Alternatively, the biological fluid can comprise ascites (e.g. in the case of pancreas, lung and colon), seminal plasma (e.g. in the case of prostate cancer), periotenal fluid (e.g. in the case of pancreas, lung and colon), pancreatic juice (e.g. in the case of pancreas), and saliva (in the case of lung cancer).

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “specifically binds” as used herein refers to a binding reaction that is determinative of the presence of the biomarker (e.g. polypeptide or nucleic acid) often in a heterogeneous population of macromolecules. For example, when the biomarker specific reagent is an antibody, specifically binds refers to the specified antibody binding with greater affinity to the cognate antigenic determinant than to another antigenic determinant, for example binds with at least 2, at least 3, at least 5, or at least 10 times greater specificity; and when a probe, specifically binds refers to the specified probe under hybridization conditions binds to a particular gene sequence at least 1.5, at least 2 at least 3, or at least 5 times background.

The term “soluble biomarker” as used herein refers to a polypeptide biomarker gene expression product or fragment thereof that is detectable in a biological fluid such as ascites or blood or a fraction thereof, such as serum or plasma. For example, a soluble biomarker includes a polypeptide that is secreted, released, or shed from a cell and detectable in for example serum.

The term “subject” as used herein refers to any member of the animal kingdom, preferably a human being.

The phrase “therapy” or “treatment” as used herein, refers to an approach aimed at obtaining beneficial or desired results, including clinical results and includes medical procedures and applications including for example chemotherapy, pharmaceutical interventions, surgery, radiotherapy and naturopathic interventions as well as test treatments for treating cancer. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “tissue specific” as used herein means that it is predominantly expressed in a single tissue or related tissue, for example expressed at a level of at least 2 fold, at least 4 fold, at least 6 fold or at least 10 fold greater compared to an unrelated tissue (e.g. from a different organ, of a different origin and/or comprising different cell types, e.g. epithelial, mesenchymal etc). As demonstrated in the Examples, proteins considered tissue specific were typically expressed in less than 20% of tissues examined. For each tissue, proteins with expression profiles showing similar values of expression in, or strong expression in, more than the selected tissue were eliminated (strong expression is defined as ≧10 times the median expression value in all tissues (e.g. more than 3, more than 4 or more than 5 tissues). Moreover, for each tissue, proteins with high/strong expression in the selected tissue and medium/moderate expression (e.g. less than a 2 fold increase) in more than two other tissues were also eliminated.

The term “Resectable cancer” as used herein comprises a subset of cancers that are typically early stage cancer that can be surgically excised. Stage can be used as a proxy for example in terms of pancreatic cancer, Stages IA, IB and IIA Pancreatic Cancer are typically resectable and in the examples are used as a proxy for resectable pancreatic cancer samples. The term “Maybe Resectable” in relation to pancreatic cancer is understood to typically include for example Stage IIB Pancreatic Cancer. Typically the term “Non-resectable” is associated with stage III and IV Pancreatic Cancer.

The term “early stage cancer” as used herein means cancer prior to metastasis and/or organ extravasion. For example with respect to pancreatic cancer, early stage cancer comprises stages IA, IB and IIA.

The term “CA19-9 negative patients” as used herein refer to subjects who have a CA19-9 level that is less than 37 IU/mL and/or individuals who are Lewis^{a-b-}, which is about 5-10% of the Caucasian population. In this population CA19-9 is not appreciably expressed even in those with advanced disease.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described are intended to be applicable to other embodiments herein described for which they are suitable as would be understood by a person skilled in the art. For example, in the above passages, different aspects of the invention are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods and Uses

Recent advances in high-throughput technologies (e.g. high-content microarray chips, serial analysis of gene expression, expressed sequence tags) have enabled the creation of publicly available gene and protein databases that describe the expression of thousands of genes and proteins in multiple tissues. Five gene databases and one protein database were utilized herein to identify tissue specific biomarkers. The C-It [9, 10], Tissue-specific and Gene Expression and Regulation (TiGER) [11, 12], and UniGene [13, 14] databases are based on expressed sequence tags (ESTs). The BioGPS [15-17] and VeryGene [18, 19] databases are based on microarray data. The Human Protein Atlas (HPA) [20, 21] are based on immunohistochemistry (IHC) data.

Diamandis et al. have previously characterized the proteomes of conditioned media (CM) from 44 cancer cell lines and three near normal cell lines and 11 relevant biological fluids (e.g., pancreatic juice and ascites) using multi-dimensional liquid chromatography tandem mass spectrometry, identifying between 1000-4000 proteins per cancer site [22-33, unpublished data].

Numerous candidate biomarkers have been identified from in silico mining of gene-expression profiling [34-36] and the HPA [37-48]. Described herein is a strategy to identify tissue-specific proteins using publicly available gene and protein databases. The strategy mines databases for proteins highly specific to or strongly expressed in one tissue, selects proteins, which are secreted or shed, and integrates proteomic datasets enriched for the cancer secretome to prioritize candidates for further verification and validation studies. Integrating and comparing proteins identified from databases based on different data sources (ESTs, microarray, and IHC) with the proteomes of the conditioned media of cancer cell lines and relevant biological fluids will minimize the shortcomings of any one source, resulting in the identification of more promising candidates.

Tissue-specific proteins were identified as candidate biomarkers for colon, lung, pancreatic, and prostate cancer. The strategy described can be applied to identify tissue-specific proteins for other cancer sites. Colon, lung, pancreatic, and prostate cancer are ranked among the top leading causes of cancer-related deaths, cumulatively accounting for an estimated half of all cancer-related deaths [50]. Early diagnosis is essential for improving patient outcomes as early-stage cancers are less likely to have metastasized and are more amenable to curative treatment. The five-year survival rate when treatment is administered on organ-confined cancer compared to metastatic stages drops dramatically from 91% to 11% in colorectal cancer, 53% to 4% in lung cancer, 22% to 2% in pancreatic cancer, and 100% to 31% in prostate cancer [50].

Forty-eight tissue-specific proteins were identified as candidate biomarkers for the selected tissue types.

Accordingly, an aspect of the disclosure includes a method of identifying a candidate cancer biomarker comprising:
a. querying one and preferably two or more protein databases;
b. identifying one or more putative biomarkers that are tissue specific and/or have increased expressed in the tissue compared to at least 5 other tissues;
c. querying for each of one or more putative biomarkers, at least one nucleic acid database to confirm transcript of putative biomarker is tissue specific and/or has increased expression in the tissue compared to at least 5 other tissues;
d. selecting tissue specific putative biomarkers that are determined to be tissue specific and/or has increased expression compared to at least 5 other tissues in one or more of the queried protein databases and one or more of the nucleic acid databases according to selected thresholds;
e. optionally determining if a tissue specific putative biomarker is likely a soluble protein for example a transmembrane and/or shed protein; and
f. selecting one or more tissue specific putative biomarkers, optionally soluble tissue specific putative markers as a candidate cancer biomarker.

Using the strategy, a number of candidate biomarkers were identified. As described, 14 of the identified set, which were selected according to the described parameters, included known biomarkers. Further, CUZD1 was validated and shown to discriminate pancreas cancer samples from control benign samples as well as to differentiate different stages of pancreatic cancer.

Also described is identification of several candidate biomarkers through differential tissue proteomic analysis of pancreatic adenocarcinoma and adjacent normal tissues. DSP, LAMC2, GP73 and DSG2 were identified as candidates and LAMC2 and DSG2 were validated as biomarkers capable of discriminating between healthy normal and pancreatic cancer patients. LAMC2 appeared significantly elevated in the sera of pancreatic cancer patients

As described in the Examples, colon, lung, pancreas and prostate tissue specific candidate biomarkers were identified.

Another aspect of the disclosure includes a method of validating a candidate biomarker as a cancer biomarker comprising:
a. selecting a candidate biomarker from the group consisting of CEACAM7, CLCA1, GPA33, LEFTY1, ZG16, IRX5, LAMP3, MFAP4, SCGB1A1, SFTPA2, SFTPB, SFTPC, SFTPD, TMEM100, AQP8, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3, DSP, LAMC2, GP73 and/or DSG2;
b. measuring an amount of the selected candidate biomarker in a plurality of samples obtained from a plurality of subjects with cancer wherein the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; the cancer is pancreas cancer if AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2 is selected; or the cancer is prostate cancer if NPY, PSCA, RLN1 and/or SLC45A3 is selected;
c. comparing to a control;
d. identifying an increase in the amount of the selected candidate biomarker in the sample as compared to the control; and
e. identifying a statistically significant increase in the amount of the selected candidate biomarker in the sample as compared to the control;
   wherein a statistically significant increased amount of the selected biomarker in the plurality of samples compared to the control is indicative the selected candidate biomarker is a cancer biomarker.

In an embodiment, the sample is a cell or tissue sample comprising cancer cells. For example, the sample can be a fresh tissue, frozen cells/tissue and/or fixed cells/tissue including formalin fixed, paraffin embedded (FFPE) samples. The sample can be a biopsy. In an embodiment, the sample comprises a biological fluid, such as blood or a fraction thereof such as serum or plasma.

The strategy disclosed can comprise a step of selecting for soluble biomarkers. Accordingly a further aspect includes a method of validating a candidate biomarker as a soluble cancer biomarker comprising:
a. selecting a candidate biomarker from the group consisting of CEACAM7, CLCA1, GPA33, LEFTY1, ZG16, IRX5, LAMP3, MFAP4, SCGB1A1, SFTPA2, SFTPB, SFTPC, SFTPD, TMEM100, AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3, DSP, LAMC2, GP73 and/or DSG2;
b. measuring an amount of the selected candidate in a plurality of biological fluid samples obtained from a plurality of subjects with cancer, wherein the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; the cancer is pancreas cancer if AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2 is selected; or the cancer is prostate cancer if NPY, PSCA, RLN1 and/or SLC45A3 is selected;
c. comparing to a control; and
d. identifying an increase in the amount of selected candidate biomarker in the biological fluid as compared to the control;
   wherein a statistically significant increased amount of the selected biomarker in the plurality of biological fluid samples compared to the control is indicative the selected candidate biomarker is a soluble cancer biomarker.

In an embodiment, the biological fluid is selected from blood or a fraction thereof. In an embodiment, the fraction thereof is serum or plasma.

In an embodiment, the biological fluid is blood or a a blood fraction such as serum or plasma (e.g. in the case of pancreas, colon, lung and prostate). Alternatively, the biological fluid can comprise ascites (e.g. in the case of pancreas, lung and colon), seminal plasma (e.g. in the case of prostate cancer), periotenal fluid (e.g. in the case of pancreas, lung and colon), pancreatic juice (e.g. in the case of pancreas), and saliva (in the case of lung cancer).

For example when the sample is blood or a fraction thereof such as plasma, an ACD (anticoagulant) vacutainer tube can be used to collect the plasma samples. Samples are in an embodiment processed within 24 hours of blood draw, when samples are not frozen. Blood samples can be centrifuged at room temperature for example for about 10 minutes (at 1000×g) to pellet the cells. Right after the centrifugation, the plasma samples can be aliquoted into cryotubes and stored at −80° C. until analysis.

In an embodiment, the biomarker is selected from CEACAM7, CLCA1, GPA33, LEFTY1, ZG16, IRX5, LAMP3, MFAP4, SCGB1A1, TMEM100, AQP8, CTRB1, CTRB2, CUZD1, KLK1, PNLIPRP1, PNLIPRP2, PRSS3, REG3G, SLC30A8, NPY, PSCA, RLN1 and/or SLC45A3.

In another embodiment, the biomarker is selected from CUZD1 and/or LAMC2.

In an embodiment, a combination of candidate biomarkers is validated, the combination comprising two or more selected biomarkers. For example, two or more biomarkers may be used in combination to provide for example increased specificity and/or sensitivity.

In an embodiment, the two or more biomarkers are selected from CEACAM7, CLCA1, GPA33, LEFTY1 and/or ZG16 and the cancer is colon cancer.

In an embodiment, the two or more biomarkers are selected from IRX5, LAMP3, MFAP4, SCGB1A1, SFTPC and/or TMEM100 and the cancer is lung cancer.

In an embodiment, the two or more biomarkers are selected from AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2 and the cancer is pancreas cancer.

In an embodiment, the two or more biomarkers are selected from NPY, PSCA, RLN1 or SLC45A3 and the cancer is prostate cancer.

As disclosed herein, CUZD1 was validated and shown to be useful for discriminating subjects with pancreas cancer and subjects without. LAMC2 and DSG2 were also validated. In particular, CUZD1 and LAMC2 demonstrated strong diagnostic ability individually, retained diagnostic accuracy in CA19.9 negative PDAC cases, and multi-parametric models demonstrated complementarity of CUZD1 and/or LAMC2 with CA19.9, including for example in the detection of early stage PDAC (stages IA, IB, IIA and IIB) from benign conditions.

Accordingly, in an embodiment the method further comprises using a validated cancer biomarker for evaluating a probability a subject has cancer and/or as a diagnostic to diagnose a cancer.

Accordingly a further aspect provides a method of evaluating a probability a subject has cancer and/or diagnosing the subject with cancer, the method comprising:
a. measuring an amount of a biomarker from the group consisting of CEACAM7, CLCA1, GPA33, LEFTY1, ZG16, IRX5, LAMP3, MFAP4, SCGB1A1, SFTPC, TMEM100, AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3, DSP, LAMC2, GP73 and/or DSG2 in a sample from a subject with cancer; wherein the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; the cancer is pancreas cancer if AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2 is selected; or the cancer is prostate cancer if NPY, PSCA, RLN1 and/or SLC45A3 is selected;
b. comparing the measured amount to a control and detecting an increase in the amount of the biomarker compared to control and
c. identifying the subject as having or having an increased probability of having the cancer when an increase in the biomarker compared to control is detected.

Also provided in another aspect, is use of a biomarker selected from the group consisting of CEACAM7, CLCA1, GPA33, LEFTY1, ZG16, IRX5, LAMP3, MFAP4, SCGB1A1, SFTPC, TMEM100, AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3 DSP, LAMC2, GP73 and/or DSG2 for evaluating if a subject has cancer and/or diagnosing cancer, wherein the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; the cancer is pancreas cancer if AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2 is selected; or the cancer is prostate cancer if NPY, PSCA, RLN1 and/or SLC45A3 is selected.

In an embodiment, the biomarker is or has been validated for example according to a method described herein.

In an embodiment, the evaluation is for diagnostic and prognostic and/or disease monitoring.

Several colon specific biomarkers were identified. In an embodiment, the colon cancer specific biomarker is selected from CEACAM7, CLCA1, GPA33, LEFTY1 and/or ZG16.

Several lung specific biomarkers were identified. In an embodiment, the lung cancer specific biomarker is selected from IRX5, LAMP3, MFAP4, SCGB1A1, SFTPC and/or TMEM100. Several pancreas specific biomarkers were identified. In an embodiment, the pancreatic cancer specific biomarker is selected from AQP8, CELA3B, CTRB1, CTRB2, CUZD1, GCG, IAPP, INS, KLK1, PNLIPRP1, PNLIPRP2, PPY, PRSS3, REG3G and/or SLC30A8. In another embodiment, the biomarker is selected from CUZD1 and/or LAMC2

Several prostate specific biomarkers were identified. In an embodiment, the prostate cancer specific biomarker is selected from KLK3, NPY, PSCA, RLN1 and/or SLC45A3. In an embodiment, the biomarker is CUZD1.

In an embodiment, the biomarker is LAMC2.

In another embodiment, the biomarker is DSG2.

As mentioned CUZD1 and LAMC2 demonstrated strong diagnostic ability individually, retained diagnostic accuracy in CA19.9 negative PDAC cases.

In an embodiment, the subject being evaluated and/or diagnosed for pancreatic cancer is CA19.9 negative.

CUZD1 and LAMC2 were able to distinguish and predict benign cases from pancreatic cancer cases. Accordingly in an embodiment, the control comprises a sample or samples of—or cut-off value derived from—benign nonpancreatic cancer illnesses, including for example chronic pancreatitis, pancreatic cyst, PD dilation and/or other benign conditions.

In addition, CUZD1 and LAMC2 were able to distinguish early from late stage pancreatic cancer.

Accordingly in an embodiment, the method comprising measuring CUZD1 and/or LAMC2 is for detecting early stage pancreatic cancer. In an embodiment, the method or use is for determining pancreatic cancer stage (e.g. early stage IA, IB or IIA; late stage can be stage III or IV) or pancreatic cancer resectabilty, and detecting a level of CUZD1 and/or LAMC2 below a control (e.g. where the control is for example derived from distinguishing early and late stage pancreatic cancer) is indicative of early stage and/or resectable cancer and above the control late stage or unresectable cancer. The control can for example be derived from comparing benign and early stage cancers. In such cases, above the cut-off distinguishing control from early stage would identify early stage pancreatic if for example below a second cutoff based on late stage pancreatic cancer.

Multi-parametric models demonstrated complementarity of CUZD1 and/or LAMC2 with CA19.9, including for example in the detection of early stage PDAC (stages IA, IB, IIA and IIB) from benign conditions.

In an embodiment, the cancer is early stage cancer. In an embodiment, pancreatic cancer is early stage pancreatic cancer. In

Two or more biomarkers of the disclosure can be assessed together. In an embodiment, 2, 3, 4, 5, 6, 7, 8, 9, 10 or more biomarkers are assessed.

Multi-parametric models for combinations of markers can be used. Estimated coefficients of the model can be used to construct a combined score for each observation which is then used for the evaluation of the multi-parametric model. For example as described in Example 8, the 3 linear models evaluated for diagnostic performance in that Example are: (1) CA19.9+11.84·CUZD1, (2) CA19.9+0.202·LAMC2, (3) CA19.9+12.41·CUZD1+0.14·LAMC2.

In addition, a biomarker for example used in clinic and/or known in the art can be combined to improve diagnostic efficacy. For example, it is demonstrated that improved and up to 100% specificity could be obtained (for example see Examples 3 and 8) when CUZD1 and known biomarker CA19.9 were assessed together. Accordingly, in an embodiment, the method further comprises measuring the amount of an additional biomarker in the sample (e.g. in addition to a biomarker of the disclosure for example as listed in Tables 5-8 and/or 11).

In an embodiment, the additional biomarker is selected from CA19.9 CEA, CYFRA-21-1 NSE TPA, proGRP, SCC, CA125 and PSA. In an embodiment, the additional biomarker is CA19.9.

In an embodiment, the additional biomarker is selected from SFTPA2, SFTPB, SFTPD, CEL, CELA2A, CPA1, CPA2, CPB1, PNLIP, PRSS1, SYCN, ACPP, FOLH1, KLK2 and/or KLK3.

In an embodiment, the biomarker is CUZD1 and the additional biomarker is CA19.9.

In an embodiment, the biomarker is LAMC2 and the additional biomarker is CA19.9

In an embodiment, the biomarker is DSG2 and the additional biomarker is CA19.9.

In an embodiment, the method comprises measuring the level of CUZD1, LAMC2 and CA19.9.

As CUZD1 and LAMC2 are able to distinguish benign from early stage and early late from late stage pancreatic cancer, the markers can be useful for monitoring cancer.

Accordingly another aspect includes a method of monitoring pancreatic cancer progression, the method comprising:
a. obtaining a test sample from the subject,
b. measuring an amount of CUZD1 and/or LAMC2 in the test sample;
c. comparing the amount of CUZD1 and/or LAMC2 in the test sample to amount of CUZD1 and/or LAMC2 in a base-line sample for the subject; and
d. identifying a difference in the amount of the CUZD1 and/or LAMC2 between the two samples;
   wherein an increase in CUZD1 and/or LAMC2 in the test sample compared to the base-line sample is indicative of progression and a decrease in CUZD1 and/or LAMC2 is indicative of improvement.

The method can be employed to monitor treatment efficacy and/or recurrence. The base line sample can be any suitable comparator that is taken before the test sample, including for example before surgery, before treatment, or during treatment that is before the subsequent sample. The base line sample can be compared to a sample obtained during remission or stable disease to assess recurrence or disease worsening.

As further explained for example in Example 2 and 8, a cut off level can be determined and chosen. The cut off level can be chosen to provide a specific specificity and/or sensitivity. In an embodiment, the specificity is selected to be at least 80%, at least 85% or at least 90%. In another embodiment, the sensitivity is selected to be at least 80%, at least 85% or at least 90%. The specificity and/or sensitivity is in an embodiment between 70% and 99% or any 0.1 increment between and including 70% and 99%.

As an example, an optimized cutoff for each marker can be obtained by minimizing the total prediction error, using for example the following formula: \sqrt{(1−sensitivity)²+(1−specificity)²}.

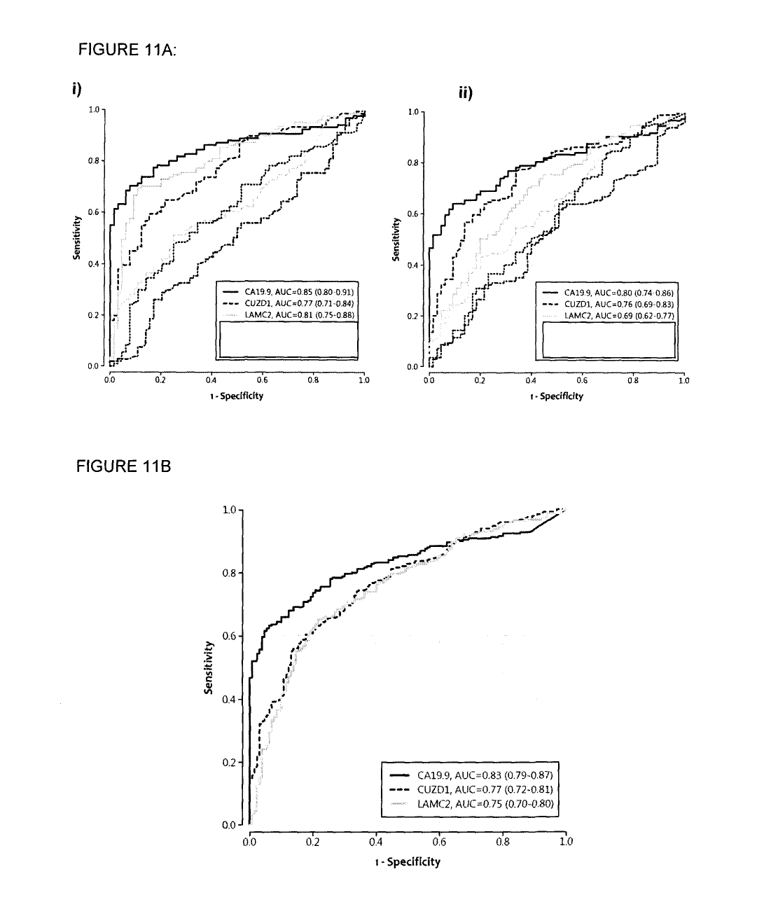

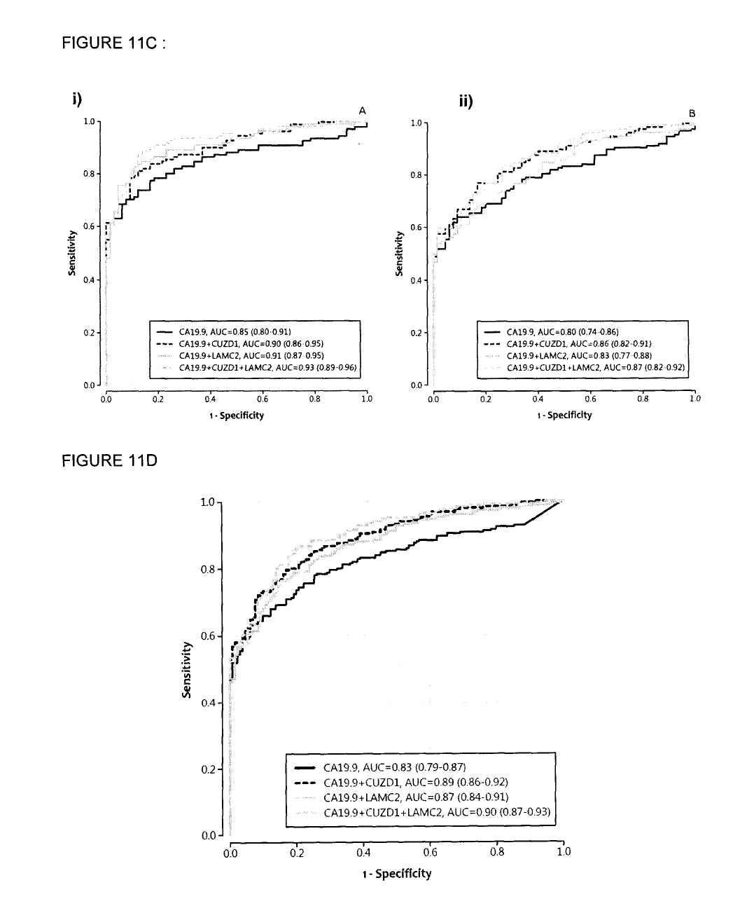

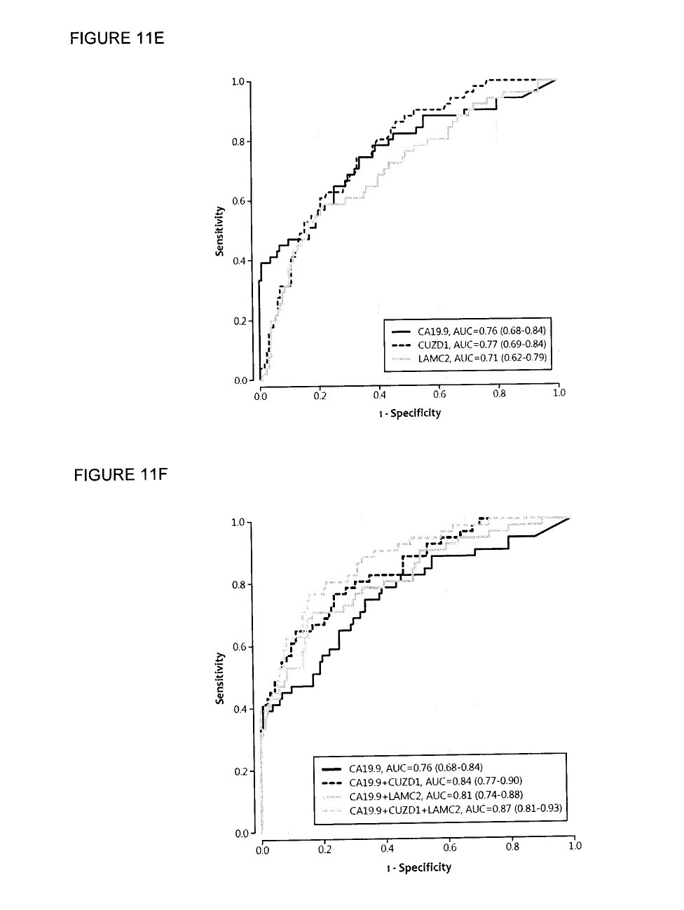

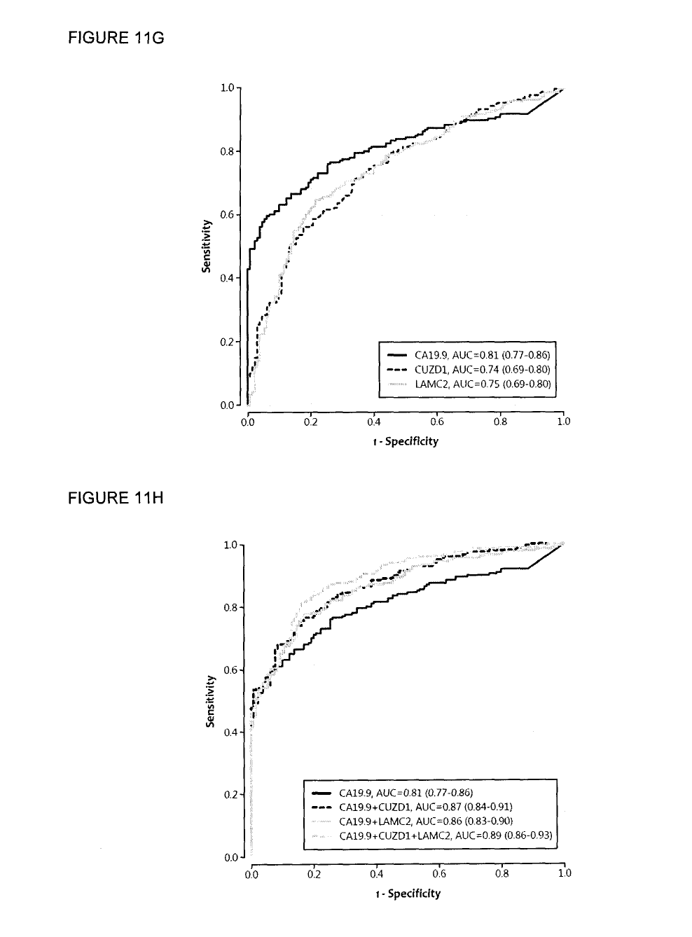

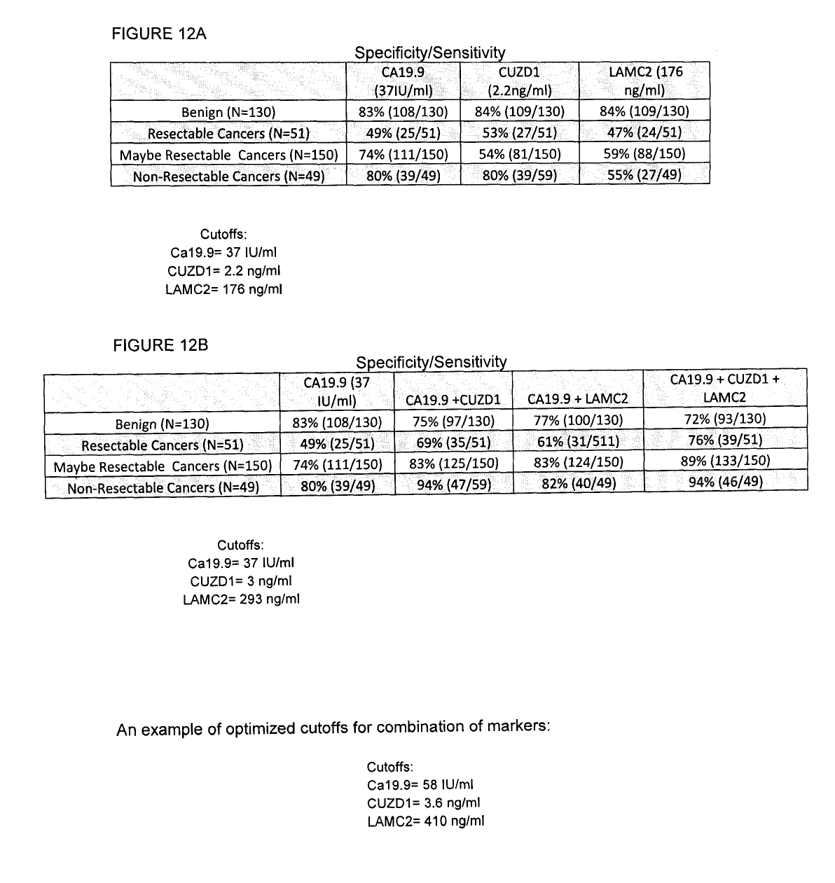

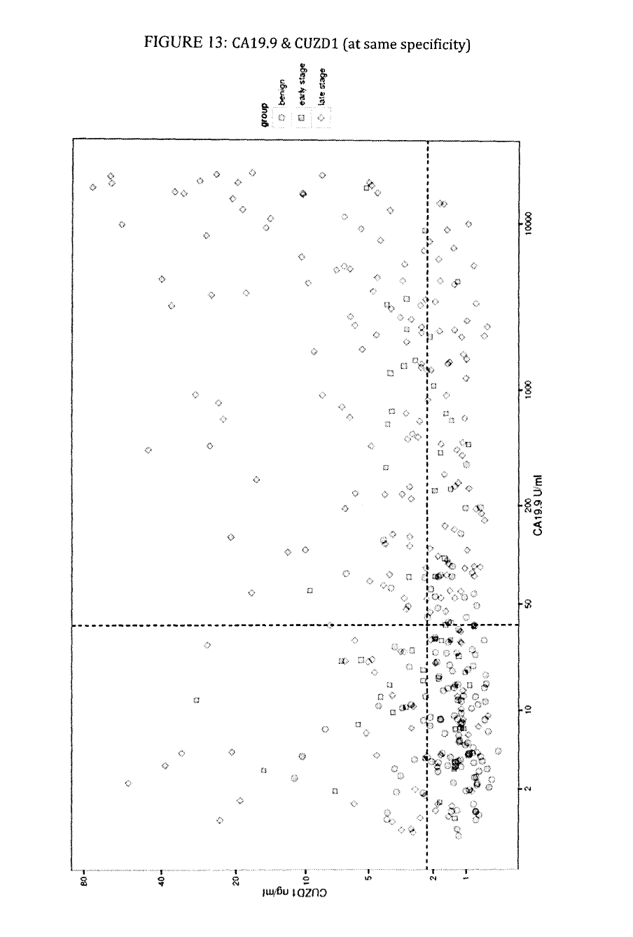

Cutoffs can be chosen based on the shortest distance of the ROC curve to the top-left corner. For example as described in Example 8, ROC curve showed the optimum diagnostic cutoff for CA19.9 was 20.3 U/mL, (area under the curve AUC 0.85, 95% CI 0.80-0.91, sensitivity 77.5%, specificity 83.1%; (FIG. 11A, Table 13). The optimum cutoff for CUZD1 was 1.8 ng/mL (AUC 0.77, 95% CI 0.71-0.84, sensitivity 64.9%, specificity 78.5%) and for LAMC2 was 123.2 ng/mL (AUC 0.81, 95% CI 0.75-0.88, sensitivity 70.3%, specificity 87.7%). Individually, CA19.9 had the greatest AUC in training and validation cohorts (FIG. 11A, Table 13). However, 22 out of 130 patients (approximately 17%) with benign disease were false positives with elevated CA19.9 levels (>37 IU/mL), limiting the specificity of CA19.9.

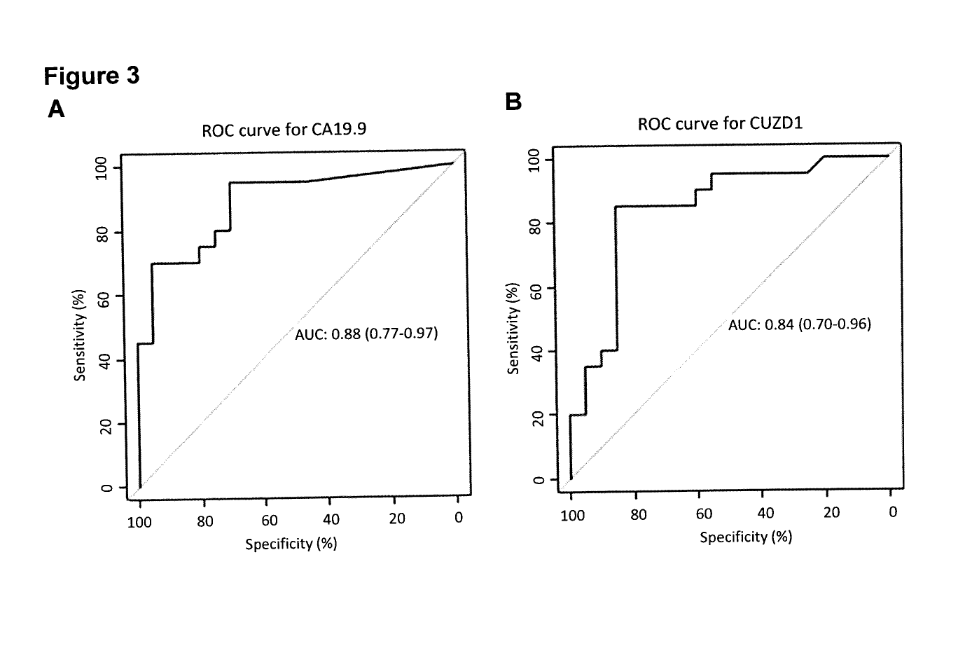

For example, a cut off level of 3.1 ng/ml was selected for CUZD1 in Example 2. Other cut off levels examined include for example 1.8 ng/mL (Example 8), 2.2 ng/mL (e.g. FIG. 3), 4.6 ng/mL 5 ng/mL This value can correspond to the mean concentration of CUZD1 protein corrected for dilution using an ELISA assay. A person skilled in the art would recognize that the cut-off level would vary for example with the method of detection, sample type, sample preparation (e.g. dilution) etc.

In an embodiment, the amount of CUZD1 indicative for cancer is greater than 3.1 ng/ml mean concentration (in the absence of a very optimized immune-assay the cutoff value can range between 1.5 ng/ml up to approx. 10 ng/ml/. In an embodiment, cutoff value for CUZD1 in the diagnosis of pancreatic cancer is about 2 to about 5 ng/ml.

In an embodiment, the amount of CUZD1 indicative the subject has pancreatic cancer or an increased probability of pancreatic cancer is greater than about 2 ng/ml, 2.2 ng/ml, 2.4 ng/ml, 2.6 ng/ml, 2.8 ng/ml, 3 ng/ml, 3.1 ng/ml, 3.2 ng/ml, 3.4 ng/ml, 3.6 ng/ml, 3.8 ng/ml, 4 ng/ml, 4.2 ng/ml, 4.4 ng/ml, 4.6 ng/ml, 4.8 ng/ml, 5 ng/ml.

A person skilled in the art would recognize that the control and/or cut-off level selected can vary, for example according to the method employed eg to evaluate a probability, diagnose, monitor disease or treatment efficacy as well as the number of biomarkers being assessed.

Cut-off levels were also determined for LAMC2. For example a cut off level of 150 ng/ml is used for example in Example 8.

In an embodiment, the amount of LAMC2 indicative the subject has pancreatic cancer or an increased probability of pancreatic cancer is greater than about 100 ng/ml, 120 ng/ml, 140 ng/ml. 160 ng/ml, 170 ng/ml, 180 ng/ml, 200 ng/ml, 220 ng/ml, 240 ng/ml, 260 ng/ml, 280 ng/ml, 300 ng/ml, 320 ng/ml, 340 ng/ml, 360 ng/ml, 380 ng/ml or 400 ng/ml.

The cut-off can also be based on fold increase. In an embodiment, the level of biomarker in the sample is at least 1.5 fold, 2 fold, 3 fold, 4 fold, 5 fold, 6 fold, 7 fold, 8 fold, 9 fold, 10 fold, 11 fold, 12 fold or at least 15 fold increased compared to the control.

The methods can be combined with conventional methods. For example, the methods can be combined and/or confirmed with conventional cancer imaging methods. For example, conventional imaging tools that can be used for example to diagnose pancreatic cancer include computerized tomography (CT) scanning, magnetic resonance imaging (MRI), endoscopic ultrasonography (EUS), and endoscopic retrograde cholangiopancreatography (ERCP). These methods can be costly and/or invasive but are powerful in tumour staging and confirming a suspected pancreatic mass. In an embodiment, CUZD1, LAMC2 optionally in combination with CA19.9 are measured and when an increase amount compared to a control is detected, the method further comprises follow up testing with a conventional imaging tool or other diagnostic method.

A person skilled in the art would recognize that a number of methods can be used to measure the level of a polypeptide biomarker. In an embodiment, the measuring comprises an immunoassay, for example immunohistochemistry, ELISA, Western blot, immunoprecipation and the like, where a biomarker detection agent such as an antibody for example, a labeled antibody, is contacted with the sample specifically binds the biomarker and permits for example relative or absolute ascertaining of the amount of polypeptide biomarker.

In an embodiment, the method comprises incubating the sample with a first antibody specific for the biomarker which is directly or indirectly labeled with a detectable substance and a second antibody specific for the biomarker which is immobilized; separating and removing unbound first antibody from the second antibody; and determining the amount of biomarker by measuring the detectable substance.

Each biomarker is detected by an antibody that binds specifically to the biomarker. In an embodiment, each antibody is independently selected from the group consisting of a monoclonal antibody, a polyclonal antibody, immunologically active antibody fragment, humanized antibody, an antibody heavy chain, an antibody light chain, a genetically engineered single chain Fv molecule, or a chimeric antibody.

For nucleic acid biomarker embodiments, hybridization and PCR protocols where a probe or primer or primer set are used to ascertain the amount of nucleic acid biomarker can be used, including for example probe based and amplification based methods including for example microarray analysis, RT-PCR such as quantitative RT-PCR, serial analysis of gene expression (SAGE), Northern Blot, digital molecular barcoding technology, for example Nanostring nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells.

In an embodiment, the method is for early detection of cancer.

Another aspect includes an array that comprises probes for detecting one or more biomarkers of the disclosure and optionally additional biomarkers. In an embodiment, the array comprises probes for detecting one or more or all of the biomarkers listed in Table 5, 6, 7, 8 and/or 11.

Also provided in another aspect is a kit which can be for use in a method or use described herein. In an embodiment, the kit comprises one or more of: a biomarker specific reagent for a biomarker of the disclosure and optionally an additional biomarker; a kit standard; instructions for use and a vial housing the biomarker specific reagent and/or kit standard.

In an embodiment, the kit comprises two or more antibodies. In an embodiment, the two or more antibodies comprise an antibody specific for CUDZ1 and an antibody specific for CA19.9.

In another embodiment still, the kit further comprises reagents for qRT-PCR, including buffers, reverse transcription and amplification primers for the target genes and endogenous control genes, and control RNA from normal oral tissue.

In another embodiment, the kit further comprises reagents for digital molecular barcoding technology, including for example buffers, hybridization solution, and/or one or more labeled probes.

The kit can optionally comprise sample collection tubes and/or assay plates for conducting one or more assays.

In an embodiment, the kit comprises a kit standard, and at least one biomarker specific agent that can measure or be used in an assay to measure an expression level of a biomarker selected from biomarkers listed in Table 4 and/or 11, or optionally a biomarker listed in Tables 5, 6, 7, 8 and/or 11.

In an embodiment, the kit standard is a quantity of a biomarker for use as a standard.

In another embodiment, the kit standard is an RNA control such as reference RNA.

In an embodiment, the kit comprises an array comprising a plurality of biomarker detection agents for detecting one or more biomarkers listed in Table 4, or optionally Tables 5, 6, 7, 8 and/or 11.

In an embodiment, the kit comprises a sample collection vessel for example a vacutainer tube or other sterile tube for biological fluid (e.g. blood) collection. The sample collection vessel can be uniquely numbered or comprise other identifier. The kit can include instructions, for example stipulating the how to use the kit with a method disclosed herein and/or instructions for obtaining and sending the sample for assessment as well as how to retrieve from an electronic database, the result of the test and/or prognosis

In an embodiment, the kit is a diagnostic kit.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present application:

### EXAMPLES

### Example 1

### Background

There is an important need for the identification of novel serological biomarkers for the early detection of cancer. Current biomarkers suffer from a lack of tissue-specificity, rendering them vulnerable to non-disease-specific increases. The present study details a strategy to rapidly identify tissue-specific proteins using bioinformatics.

### Methods

Previous studies focus on either gene or protein expression databases for the identification of candidates. An strategy was developed that mines six publicly available gene and protein databases for tissue-specific proteins, selects proteins likely to enter the circulation, and integrates proteomic datasets enriched for the cancer secretome, to prioritize candidates for further verification and validation studies.

### Results

Using colon, lung, pancreas, and prostate cancer as case examples, 48 candidate tissue-specific biomarkers were identified.

### Conclusions

A novel strategy using bioinformatics to identify tissue-specific proteins that are potential cancer serum biomarkers is described further in Examples below.

### Example 2

### Methods

### in Silico Discovery

Seven gene and protein databases were mined to identify proteins highly specific to or strongly expressed in one tissue. Colon, lung, pancreatic, and prostate tissues were examined.

Each tissue was searched in the C-It database [10] for proteins enriched in the selected tissue (human data only). Since the C-It database did not have colon data available, only lung, pancreas, and prostate tissue were searched. Literature information search parameters of fewer than five publications in PubMed and fewer than three publications with the Medical Subject Headings (MeSH) term of the searched tissue were used. The option of adding z-scores of the corresponding SymAtlas microarray probe sets to the protein list was included [16]. Only proteins with a corresponding SymAtlas z-score of ≧|1.96|, corresponding to a 95% confidence level of enrichment, were included in the lists. Proteins without a SymAtlas z-score were ignored. The TiGER database [12] was searched for proteins preferentially expressed in each tissue based on ESTs by searching each tissue using ‘Tissue View’. The UniGene database [14] was searched for tissue-restricted genes using the following search criteria: [tissue][restricted]+“Homo sapiens”, for the lung, pancreas, and prostate tissues. Since the UniGene database did not have data for the colon tissue, a search of: [colorectal tumor][restricted]+“Homo sapiens” was used.

The BioGPS database (v. 2.0.4.9037) [17] plugin ‘Gene expression/activity chart’ using the default human data set ‘GeneAtlas U133A, gcrma’ [16] was searched with a protein whose gene expression profile using the BioGPS plugin showed it to be specific to, and strongly expressed in, one tissue of interest. Chloride channel accessory 4 (CLCA4), surfactant protein A2 (SFTPA2), pancreatic lipase (PNLIP), and kallikrein-related peptidase 3 (KLK3) were selected for colon, lung, pancreas, and prostate tissues, respectively. For each protein searched, a correlation cutoff of 0.9 was used to generate a list of proteins with a similar expression pattern to the initial protein searched. Each tissue was searched in the VeryGene database [19] using ‘Tissue View’ for tissue-selective proteins.

The HPA [21] was searched for proteins strongly expressed in each normal tissue with annotated expression. Annotated protein expression is a manually curated score based on IHC staining patterns in normal tissues from two or more paired antibodies binding to different epitopes of the same protein, which describes the distribution and strength of expression of each protein in cells [51].

### Identification of Protein Overlap in Databases

An in-house developed Microsoft Excel macro was utilized to evaluate the number of times a protein was identified in each tissue and which database had identified it. Proteins identified in only one database were eliminated. Proteins identified in databases could represent more promising candidates at this stage, since databases based on varying sources of data identified the protein as being highly specific to or strongly expressed in one tissue.

### Secreted or Shed Proteins

For each tissue type, the list of proteins identified in ≧2 databases was exported into a comma-delimited Microsoft Excel file. An in-house secretome algorithm [Karagiannis G S et al., unpublished] was applied to identify proteins that are either secreted or shed. The secretome algorithm designates a protein as secreted or shed if it is either predicted to be secreted based on the presence of a signal peptide, or through non-classical secretion pathways, or predicted to be a membranous protein based on amino-acid sequences corresponding to transmembrane helices. Proteins that were not designated as secreted or shed were eliminated.

### Verification of in Silico Expression Profiles

The BioGPS and HPA databases were used to manually verify the expression profiles of the proteins identified as being secreted or shed, for strength and specificity of expression. The BioGPS database was chosen above the other gene databases as it offers a gene expression chart and the ability to batch search for a list of proteins, which allowed efficient searching and verification of protein lists. If expression profiles were not available in the BioGPS database, the protein was eliminated.

The BioGPS database plugin ‘Gene expression/activity chart’ using the default human data set ‘GeneAtlas U133A, gcrma’ was searched for each protein. For each tissue, proteins with gene expression profiles showing similar values of expression in, or strong expression in, more than the selected tissue were eliminated (strong expression is defined as ≧10 times the median expression value in all tissues). In BioGPS, the color of the bars in the ‘Gene expression/activity chart’ reflects a grouping of similar samples, based on global hierarchical clustering. If strong expression was seen in more than the selected tissue, but only in tissues with the same bar color, the protein was not eliminated.

The HPA was searched for each protein, and the ‘Normal Tissue’ expression page was evaluated. Tissue presentation order by organ was selected. Preference for the evaluation of the protein's expression in normal tissue was based on the level of annotated protein expression and if annotated expression was not available, evaluation was based on the level of antibody staining. The levels of annotated protein expression are none, low, medium, and high and the levels of antibody staining are negative, weak, moderate, and strong. For each tissue, proteins with high/strong expression in the selected tissue and medium/moderate expression in more than two other tissues were eliminated. Proteins with high/strong or medium/moderate expression in more than the one selected tissue were eliminated. Proteins with low/weak or none/negative expression in the selected tissue were eliminated. If the high/strong and/or medium/moderate was seen in more than the one selected tissue, where the other tissues are in the same organ, and low/weak and/or none/negative expression in all other tissues, the protein was included.

Proteins with pending HPA data were evaluated based on their gene expression profiles. Proteins whose HPA protein expression profiles fit the criteria for elimination but whose gene expression profiles did not fit the criteria for elimination, were eliminated.

### Literature Search

The PubMed database was manually searched for each of the proteins whose expression profile was verified in silico. For each tissue, proteins that had been previously studied as candidate cancer or benign disease serum biomarkers in the selected tissue were identified. Proteins with high abundance in serum (>5 μg/mL) or known physiology and expression were eliminated.

### Proteomic Datasets

An in-house developed Microsoft Excel macro was utilized for comparison of the remaining protein lists against previously characterized in-house proteomes of the CM from 44 cancer cell lines and three near normal cell lines, and 11 relevant biological fluids [22-33, unpublished data]. Proteomes were characterized using multi-dimensional liquid chromatography tandem mass spectrometry on a linear ion trap (LTQ) Orbitrap mass spectrometer. For details, see previous publications [22-33]. The cancer cell lines were from six cancer types (breast, colon, lung, ovarian, pancreatic, and prostate cancer). The relevant biological fluids included amniotic fluid (normal, with Down Syndrome), nipple aspirate fluid, non-malignant peritoneal fluid, ovarian ascites, pancreatic ascites, pancreatic juice, pancreas tissue (normal and malignant), and seminal plasma. A complete list of cell lines and relevant biological fluids is provided in Table 1. If a protein was identified in amniotic fluid and the proteome of a tissue, this was noted but not considered as expression in a non-tissue proteome.

Moreover, the data of proteomes from the CM of 23 cancer cell lines (from 11 cancer types) was integrated, as recently published by Wu et al. [52]. Proteomes were characterized using one-dimensional SDS-PAGE and nano-liquid chromatography tandem mass spectrometry on a LTQ-Orbitrap mass spectrometer. The 11 cancer types include breast, bladder, cervical, colorectal, epidermoid, liver, lung, nasopharyngeal, oral, and pancreatic cancer, and T cell lymphoma [52]. If a protein was identified in a proteomic dataset, the proteome in which it was identified in, was noted.

### Results

### Identification of Proteins

A total of 3615 proteins highly specific to or strongly expressed in the colon, lung, pancreas, or prostate were identified by searching the databases. Searching the databases identified 976, 679, 1059, and 623 unique proteins that were highly specific to or strongly expressed in the colon, lung, pancreas, and prostate, respectively (Table 2). For the four tissue types, the C-It database identified 254 tissue-enriched proteins, the TiGER database identified 636 proteins preferentially expressed in tissue, and the UniGene database identified 84 tissue-restricted proteins. The BioGPS database identified 127 proteins similarly expressed as a protein with known tissue specificity, and the VeryGene database identified 365 tissue-selective proteins. The HPA identified 2149 proteins showing strong tissue staining and with annotated expression. A complete list of proteins identified in each tissue, by each database is summarized in Table 3.

### Protein Identification Overlap in Databases

A total of 32 proteins in the colon, 36 proteins in the lung, 81 proteins in the pancreas, and 48 proteins in the prostate were identified in ≧2 databases. Selecting for proteins identified in ≧2 databases eliminated between 92%-97% of the proteins in each of the tissue types. The majority of the remaining proteins were identified in only two of the databases, and no proteins were identified in six or all the databases. This data is summarized in Table 2.

### Secreted or Shed Proteins

The majority of the proteins identified in ≧2 databases were identified as being secreted or shed. In total, 143 of the 197 proteins from all tissues were designated as being secreted or shed (Table 2). Specifically, 26 proteins in the colon, 25 proteins in the lung, 58 proteins in the pancreas, and 34 proteins in the prostate were designated as being secreted or shed.

### Verification of in Silico Expression Profiles

Manual verification of the expression profiles of the secreted or shed proteins identified in ≧2 databases (as exemplified in the Experimental Procedures) eliminated the majority of the proteins. Twenty-one proteins in the colon, 16 proteins in the lung, 32 proteins in the pancreas, and 26 proteins in the prostate were eliminated. Only five (0.5%) of the 976 proteins initially identified as highly specific to or strongly expressed in the colon, were found to meet the filtering criteria. Nine (1.3%) of 679 proteins in the lung, 26 (2.4%) of 1059 proteins in the pancreas, and eight (1.3%) of 623 proteins in the prostate were found to meet the filtering criteria. These remaining 48 proteins are tissue-specific and secreted or shed and therefore, represent candidate biomarkers (Table 4).

### Performance of Databases

The performance of the databases was evaluated by determining how many of the 48 proteins that passed the filtering criteria were initially identified by each database. The TiGER database had been responsible for initially identifying the greatest number of proteins that passed the filtering criteria. The TiGER database, the BioGPS database, and the VeryGene database had each identified >68% of the 48 proteins. The TiGER database had identified 40 of the 48 proteins, and the BioGPS and VeryGene databases had both identified 33 of 48 proteins. The UniGene database identified 35% (17 of 48) of the proteins and the C-It database and the HPA both identified 19% (nine of 48) of the proteins (Table 4).

The accuracy of the initial protein identifications was evaluated by comparing the proportion of proteins which each database had initially identified, that passed the filtering criteria, to the total number of proteins each database initially identified. The BioGPS database showed the highest accuracy of initial protein identification. Of the proteins initially identified by the BioGPS database, 26% (33 of 127) met all the filtering criteria. The UniGene database showed 20% accuracy (17 of 84), VeryGene showed 9% (33 of 365), TiGER showed 6% (40 of 636), C-It showed 4% (9 of 254), and HPA showed 0.4% (9 of 2149).

### Literature Search

None of the colon-specific proteins had been previously studied as serum colon cancer biomarkers. Surfactant proteins have been extensively studied in relation to various lung diseases [53], and surfactant protein A2 (SFTPA2), surfactant protein B (SFTPB), and surfactant protein D (SFTPD) have been studied as serum lung cancer/lung disease biomarkers [54-56]. Elastase proteins have been studied in pancreatic function and disease [57], islet amyloid polypeptide, and pancreatic polypeptide are normally secreted [58,59] and glucagon and insulin are involved in the normal function of healthy individuals. Eight of the pancreas-specific proteins had been previously studied as serum pancreatic cancer/pancreatitis biomarkers [33,60-65]. Four of the prostate-specific proteins had been previously studied as serum prostate cancer biomarkers [66-68] (Table 4).

Protein Overlap with Proteomic Datasets

Of the tissue-specific proteins that had not been studied as serum tissue cancer biomarkers, 18 of the 26 proteins were identified in proteomic datasets (Tables 5-8). Nine proteins were exclusively identified in datasets of corresponding tissues. Of the colon-specific proteins, only glycoprotein A33 (GPA33) was identified exclusively in colon datasets. GPA33 was identified in the CM of three colon cancer cell lines (LS174T, LS180, and Colo205) [Karagiannis et al., unpublished, 52] (Table 5). None of the lung-specific proteins were identified in lung datasets (Table 6). Seven pancreas-specific proteins were exclusively identified in pancreas datasets: in the pancreatic cancer ascites [32], pancreatic juice [33], and/or normal and/or cancerous pancreatic tissue [Kosanam et al., unpublished] (Table 7). None were identified in the CM of pancreatic cancer cell lines. Neuropeptide Y (NPY) was the only prostate-specific protein identified exclusively in prostate datasets. NPY was identified in the CM of the prostate cancer cell line VCaP [Saraon et al., unpublished] and the seminal plasma proteome [25] (Table 8).

### Discussion

A strategy to identify tissue-specific biomarkers using publicly available gene and protein databases is described. Since serological biomarkers are protein-based, using only protein expression databases for the initial identification of candidate biomarkers seems more relevant. While the HPA has characterized more than 50% of human protein-encoding genes (11200 unique proteins to date), it has not completely characterized the proteome [51]. Therefore, proteins which have not been characterized by HPA but fulfill the desired criteria would be missed by searching only the HPA. There are also important limitations in using gene expression databases since there is considerable variation between mRNA and protein expression [69,70] and gene expression does not account for post-translational modification events [71]. Therefore, mining both gene and protein expression databases minimizes the limitations of each platform. To the best of the knowledge, no studies for the initial identification of candidate cancer biomarkers have been conducted using both gene and protein databases.

Initially, the databases were searched for proteins highly specific to or strongly expressed in one tissue. The search criteria were tailored to accommodate for the design of the databases, which did not allow for the simultaneous searching with both criteria. Identifying proteins that were highly specific to and strongly expressed in one tissue was considered in a later step. In the verification of the expression profiles (see Experimental Procedures), only 34% (48 of 143) of the proteins were found to meet both criteria. The number of databases mined in the initial identification can be varied at the discretion of the investigator. Additional databases will result in the same number of, or more, proteins being identified in ≧2 databases.

In the gene expression databases, the criteria used were set for maximum stringency for protein identification, to identify a manageable number of candidates. A more exhaustive search can be conducted using lower stringency criteria. The stringency could be varied in the correlation analysis using the BioGPS database plugin and the C-It database. The correlation cutoff of 0.9 used in identifying similarly expressed genes in the BioGPS database plugin could be reduced to as low as 0.75. The SymAtlas Z-Score of ≧|1.96| could be reduced to ≧|1.15|, corresponding to a 75% confidence level of enrichment. The literature information parameters used in the C-It database of fewer than five publications in Pubmed and fewer than three publications with MeSH term of the selected tissue could be reduced in stringency, to allow identification of well-studied proteins. Since C-It does not look at the content of publications in PubMed, it filters out proteins that have been studied even if they have not been studied in relation to cancer.

Although proteins which have been well-studied, but not as cancer biomarkers, represent potential candidates, in this study emphasis was on identifying novel candidates which have been, overall, minimally studied. A gene's mRNA level and protein expression can have significant variability. Therefore, if lower stringency criteria were used when identifying proteins from gene expression databases, a greater number of protein would have been identified in at least two of the databases, potentially leading to a greater number of candidate protein biomarkers identified after application of the remaining filtering criteria.

The HPA was searched for proteins strongly expressed in one normal tissue with annotated IHC expression. Annotated IHC expression was selected since it uses paired antibodies to validate the staining pattern, providing the most reliable estimation of protein expression. Approximately, 2020 of the 10100 proteins in version 7.0 of the HPA have annotated protein expression [51]. Makawita et al. [33] included the criteria of annotated protein expression when searching for proteins with ‘strong’ pancreatic exocrine cell staining for prioritization of pancreatic cancer biomarkers. A more exhaustive search could be conducted by searching the HPA without annotated IHC expression.

Secreted or shed proteins have the highest chance of entering circulation and being detected in the serum. Many groups, including the Diamandis group[23-25, 27-33], use Gene Ontology (GO) [72] protein cellular localization annotations of ‘extracellular space’ and ‘plasma membrane’ to identify a protein as secreted or shed. GO cellular annotations do not completely describe all proteins and are not always consistent with if a protein is secreted or shed. An in-house designed secretome algorithm [Karagiannis et al., unpublished data] designates a protein as secreted or shed if it is either predicted to be secreted based on the presence of signal peptide, predicted non-classical secretion or predicted as a membranous protein based on amino-acid sequences corresponding to transmembrane helices. It more robustly defines proteins as secreted or shed and was therefore used in this study.

Evaluating which of the databases had initially identified the 48 tissue-specific proteins that passed the filtering criteria, showed that the gene expression databases had identified more of the proteins than the protein expression database. The HPA had initially identified only nine of the 48 tissue-specific proteins. The low initial identification of tissue-specific proteins was due to the stringent search criteria requiring annotated IHC expression. For example, 20 of the 48 tissue-specific proteins had protein expression data available in the HPA, of which the 11 proteins that were not initially identified by HPA did not have annotated IHC expression. The expression profiles of those proteins would have passed the ‘Verification of In Silico Expression Profiles’ filtering criteria, and therefore, would have resulted in a greater initial identification of tissue-specific proteins by the HPA.

The HPA has characterized 11200 unique proteins, which is more than 50% of human protein-encoding genes [51]. Of the 48 tissue-specific proteins that met the selection criteria, only nine were initially identified from mining the HPA. Twenty of the tissue-specific proteins have been characterized by the HPA. This demonstrates the importance of combining gene and protein databases to identify candidate cancer serum biomarkers. If only the HPA was searched for tissue-specific proteins, even with lowered stringency, the 28 proteins that met the filtering criteria and represent candidate biomarkers would not have been identified.

The TIGER, UniGene, and C-It databases are based on ESTs and collectively identified 46 of the 48 proteins. Of those, only 41% (19 of the 46) were identified in of those databases. The BioGPS and VeryGene databases are based on microarray data and collectively identified 46 of the 48 proteins. Of those, 56% (26 of the 46) were identified uniquely by BioGPS and VeryGene. Clearly, even though databases are based on similar sources of data, individual databases still identified unique proteins. This demonstrates the validity of the initial approach of using databases that differently mine the same data source. The TIGER, BioGPS, and VeryGene databases collectively identified all 48 of the tissue-specific proteins. From those three databases, 88% (42 of the 48) were identified in databases, demonstrating the validity of selecting proteins identified in more than one database.

The accuracy of the databases' initial protein identification is related to how explicitly the database could be searched for the filtering criteria of proteins highly specific to and strongly expressed in one tissue. The BioGPS database had 26% accuracy, the highest, as it was searched for proteins similarly expressed as a protein of known tissue specificity and strong expression. The UniGene database, accuracy of 20%, could only be searched for proteins with tissue-restricted expression, without the ability to search for proteins also with strong expression in the tissue. The VeryGene database, accuracy of 9%, was searched for tissue-selective proteins and the TiGER database, accuracy of 6%, was searched for proteins preferentially expressed in a tissue. Their lower accuracies reflect that they could not be explicitly searched for proteins highly specific to only one tissue. The C-It database, accuracy of 4%, searched for tissue-enriched proteins and the HPA, accuracy of 0.4%, searched for proteins with strong tissue staining. These very low accuracies reflect that the search looked for proteins with strong expression in a tissue, but could not be searched for proteins highly specific to only one tissue.

The low identification of tissue-specific proteins by the C-It database is not unexpected. Given that the literature search parameters initially used, filtered out any proteins, which have ≧5 publications in PubMed, regardless of whether those publications were related to cancer, C-It only identified proteins enriched in a selected tissue which have been minimally, if at all, studied. Of the nine proteins C-It initially identified from the tissue-specific list, eight of the proteins had not been previously studied as serum candidate cancer biomarkers. Syncollin (SYCN) has only very recently been shown to be elevated in the serum of pancreatic cancer patients [33]. The eight remaining proteins C-It had identified represent especially interesting candidate biomarkers because they represent proteins that fulfill the filtering criteria but have not been well studied.

A PubMed search revealed that 14 of the 48 tissue-specific proteins identified had been previously studied or suggested as serum markers of cancer or benign disease, providing credence to the approach. The most widely used biomarkers currently suffer from a lack of sensitivity and specificity due to the fact they are not tissue-specific. CEA is a widely used colon and lung cancer biomarker. It was identified by the BioGPS and TIGER databases and the HPA as highly specific to or strongly expressed in the colon, but not by any of the databases for the lung. CEA was eliminated upon evaluating the protein expression profile in silico, since it is not tissue specific. High levels of CEA protein expression were seen in the normal tissues of the digestive tract, such as esophagus, small intestine, appendix, colon, and rectum, as well as in bone marrow, and medium levels were seen in the tonsil, nasopharynx, lung, and vagina. PSA is an established, clinically relevant biomarker for prostate cancer with demonstrated tissue-specificity. PSA was identified in the strategy as a prostate-specific protein, after passing all the filtering criteria. This provides credence to the approach since the known clinical biomarkers and the strategy filtered out the biomarkers based on tissue-specificity were re-identified.

From the list of candidate proteins that have not been studied as serum cancer or benign disease biomarkers, 18 of the 26 proteins were identified in proteomic datasets. The proteomic datasets primarily contain the CM proteomes of various cancer cell lines, as well as other relevant fluids, enriched for the secretome. For proteins that have not been characterized by the HPA, it is possible the transcripts are not translated, in which case they would represent unviable candidates. If the transcripts are translated and the protein enters circulation, it must do so at a level detectable by current proteomic techniques. Proteins that have been characterized by the HPA may not necessarily enter circulation. The identification of proteins in the proteomic datasets verifies the presence of the protein in the secretome of cancer, at a detectable level, and therefore represent viable candidates. Since cancer is a highly heterogeneous disease, the integration of multiple cancer cell lines and relevant biological fluids likely provides a more, but not necessarily complete picture of the cancer proteome.

Relaxin 1 (RLN1) is a candidate protein which was not identified in any of the proteomes but its expression was confirmed by semi-quantitative RT-PCR in prostate carcinomas [73]. Therefore, if a protein was not identified in any of the proteomic datasets it does not necessarily imply that the protein is not expressed in cancer.

The proposed strategy seeks to identify candidate tissue-specific biomarkers for further experimental studies. Using colon, lung, pancreas, and prostate cancer as case examples, a total of 26 tissue-specific candidate biomarkers were identified. Using this strategy, investigators can rapidly screen for candidate tissue-specific serum biomarkers and prioritize candidates for further study based on overlap with proteomic datasets. This strategy can be used to identify candidate biomarkers for any tissue, contingent on the data availability in the mined databases, and incorporate various proteomic datasets, at the discretion of the investigator.

### Example 3

### CUZD1

Pancreatic cancer is the fourth leading cause of cancer-related deaths and one of the most highly aggressive and lethal of all solid malignancies [50]. Because of the asymptomatic nature of its early stages, coupled with inadequate methods for early detection, the majority of patients (>75%) present with locally advanced and inoperable disease at the time of diagnosis [50]. At these advanced stages, chemotherapy, radiation, and combinatorial therapies are largely anecdotal, and less than 5% of patients survive up to five-years post diagnosis [50, 75].

One way to aid in the clinical management of cancer patients is through the use of serum biomarkers. Currently, the most widely used biomarker for pancreatic cancer is carbohydrate antigen 19.9 (CA19.9), a sialylated Lewis A antigen found on the surface of proteins [5, 76]. Although CA19.9 is elevated mainly in late stage pancreatic cancer, it is also elevated in benign diseases of the pancreas and in other malignancies of the gastrointestinal tract [77]. Other tumor markers such as members of the carcinoembryonic antigen (CEA) [78, 79] and mucin (MUC) [80-82] families have also been associated with pancreatic cancer. When used in combination, with or without CA-19.9, some of these markers have shown enhanced sensitivity and specificity; however none have become a constant fixture in the clinic. The lack of a single highly specific and sensitive marker has led to a growing consensus in the field toward the development of multiparametric panels of biomarkers, whereby the combinatorial assessment of multiple molecules can likely achieve increased sensitivity and specificity for disease detection and management [83-85].

CUZD1 [Swiss-Prot: Q86UP6] is a protein of unknown function that has homology to chimpanzee, dog, mouse, rat, and chicken. Previously, CUZD1 has been identified by immunohistochemistry in normal ovarian and ovarian tumor cells [86]. These findings suggest that CUZD1 has a role in cell motility, cell-cell interactions and/or interactions with the extracellular matrices [86].

### Discovery of CUZD1 Using an in Silico Discovery Platform

Five gene databases and one protein database were mined to identify proteins highly specific to or strongly expressed in the pancreas tissue. The C-It [9, 10], Tissue-specific and Gene Expression and Regulation (TiGER) [11, 12], and UniGene [13, 14] databases are based on expressed sequence tags (ESTs). The BioGPS [15-17] and VeryGene [18, 19] databases are based on microarray data. The Human Protein Atlas (HPA) [20, 21] is based on immunohistochemistry (IHC) data.

The C-It database [10] was searched for proteins enriched in the pancreas. Literature information search parameters of fewer than five publications in PubMed and fewer than three publications with the Medical Subject Headings (MeSH) term of the pancreas were used. The option of adding z-scores of the corresponding SymAtlas microarray probe sets to the protein list was included [16]. Only proteins with a corresponding SymAtlas z-score of ≧|1.96|, corresponding to a 95% confidence level of enrichment, were included in our lists. Proteins without a SymAtlas z-score were ignored. The TIGER database [12] was searched for proteins preferentially expressed in the pancreas based on ESTs by searching using ‘Tissue View’. The UniGene database [14] was searched for pancreas-restricted genes using the following search criteria: [pancreas][restricted]+“Homo sapiens”. The BioGPS database (v. 2.0.4.9037) [17] plugin ‘Gene expression/activity chart’ using the default human data set ‘GeneAtlas U133A, gcrma’ [16] was searched with a protein whose gene expression profile using the BioGPS plugin showed it to be specific to, and strongly expressed in, the pancreas. Pancreatic lipase (PNLIP) was selected. A correlation cutoff of 0.9 was used to generate a list of proteins with a similar expression pattern to the initial protein searched. The VeryGene database [19] was searched for pancreas-selective proteins using ‘Tissue View’. The HPA [21] was searched for proteins strongly expressed in the normal pancreas with annotated expression. Annotated protein expression is a manually curated score based on IHC staining patterns in normal tissues from two or more paired antibodies binding to different epitopes of the same protein, which describes the distribution and strength of expression of each protein in cells [51].

### Identification of Protein Overlap in Databases

An in-house developed Microsoft Excel macro was utilized to evaluate the number of times a protein was identified in the pancreas and which database had identified it. Proteins identified in only one database were eliminated. Proteins identified in ≧2 databases could represent more promising candidates at this stage, since databases based on varying sources of data identified the protein as being highly specific to or strongly expressed in one tissue.

### Secreted or Shed Proteins

The list of proteins identified in ≧2 databases was exported into a comma-delimited Microsoft Excel file. An in-house secretome algorithm [Karagiannis G S et al., unpublished] was applied to identify proteins that are either secreted or shed. The secretome algorithm designates a protein as secreted or shed if it is either predicted to be secreted based on the presence of a signal peptide, or through non-classical secretion pathways, or predicted to be a membranous protein based on amino-acid sequences corresponding to transmembrane helices. Proteins that were not designated as secreted or shed were eliminated.

### Verification of in Silico Expression Profiles

The BioGPS and HPA databases were used to manually verify the expression profiles of the proteins identified as being secreted or shed, for strength and specificity of expression. The BioGPS database was chosen above the other gene databases as it offers a gene expression chart and the ability to batch search for a list of proteins, which allowed efficient searching and verification of protein lists. If expression profiles were not available in the BioGPS database, the protein was eliminated.

The BioGPS database plugin ‘Gene expression/activity chart’ using the default human data set ‘GeneAtlas U133A, gcrma’ was searched for each protein. Proteins with gene expression profiles showing similar values of expression in, or strong expression in, more than the pancreas were eliminated (strong expression is defined as ≧10 times the median expression value in all tissues). In BioGPS, the color of the bars in the ‘Gene expression/activity chart’ reflects a grouping of similar samples, based on global hierarchical clustering. If strong expression was seen in more than the pancreas, but only in tissues with the same bar color, the protein was not eliminated.

The HPA was searched for each protein, and the ‘Normal Tissue’ expression page was evaluated. Tissue presentation order by organ was selected. Preference for the evaluation of the protein's expression in normal tissue was based on the level of annotated protein expression and if annotated expression was not available, evaluation was based on the level of antibody staining. The levels of annotated protein expression are none, low, medium, and high and the levels of antibody staining are negative, weak, moderate, and strong. Proteins with high/strong expression in the pancreas and medium/moderate expression in more than two other tissues were eliminated. Proteins with high/strong or medium/moderate expression in more than the pancreas were eliminated. Proteins with low/weak or none/negative expression in the pancreas were eliminated. If the high/strong and/or medium/moderate was seen in more than the pancreas, where the other tissues are in the same organ, and low/weak and/or none/negative expression in all other tissues, the protein was included.

Proteins with pending HPA data were evaluated based on their gene expression profiles. Proteins whose HPA protein expression profiles fit the criteria for elimination but whose gene expression profiles did not fit the criteria for elimination, were eliminated.

### Literature Search

The PubMed database was manually searched for each of the proteins whose expression profile was verified in silico. Proteins that had been previously studied as candidate pancreatic cancer or benign disease serum biomarkers were identified and excluded. Proteins with high abundance in serum (>5 μg/mL) or known physiology and expression were also eliminated. The remaining subset is presented in Tables 5-8.

### Proteomic Datasets

An in-house developed Microsoft Excel macro was utilized for comparison of the remaining protein lists against previously characterized in-house proteomes of the culture medium (CM) from 44 cancer cell lines and three near normal cell lines, and 11 relevant biological fluids [22-33, our unpublished data]. Proteomes were characterized using multi-dimensional liquid chromatography tandem mass spectrometry on a linear ion trap (LTQ) Orbitrap mass spectrometer. For details, see our previous publications [22-33]. The cancer cell lines were from six cancer types (breast, colon, lung, ovarian, pancreatic, and prostate cancer). The relevant biological fluids included amniotic fluid (normal, with Down Syndrome), nipple aspirate fluid, non-malignant peritoneal fluid, ovarian ascites, pancreatic ascites, pancreatic juice, pancreas tissue (normal and malignant), and seminal plasma.

Data of proteomes from the CM of 23 cancer cell lines (from 11 cancer types) was also integrated, as recently published by Wu et al. [52]. Proteomes were characterized using one-dimensional SDS-PAGE and nano-liquid chromatography tandem mass spectrometry on a LTQ-Orbitrap mass spectrometer. The 11 cancer types include breast, bladder, cervical, colorectal, epidermoid, liver, lung, nasopharyngeal, oral, and pancreatic cancer, and T cell lymphoma [52]. If a protein was identified in a proteomic dataset, the proteome in which it was identified in, was noted.

### Results

### Validation of CUZD1 as a Serum Pancreatic Biomarker

Both CA19.9 and CUZD1 were quantified in serum with commercially available ELISA kits (Roche and USCN, respectively) as per the manufacturer's recommendations.

Validation of CA19.9 (for comparison) and CUZD1 was performed using 20 benign, pancreatic cyst serum samples and 20 pancreatic cancer samples of mixed stages. At a cutoff of 37 IU/mL, CA19.9 showed 70% specificity and 80% sensitivity (identified six false positives, shown in Table 9 in squares, four false negatives, shown in circles). At a cutoff of 3.1 ng/mL, CUZD1 showed 85% specificity and 85% sensitivity (identified three false positives, shown in Table 9 in squares, and three false negatives, shown in circles). CUZD1 had a similar area under the curve (AUC) value to CA19.9 (Table 10, FIG. 3). Only two of the six samples which CA19.9 identified as false positives were also identified as false positives by CUZD1. None of the samples which CA19.9 identified as false negatives were identified as false negatives by CUZD1. Based on these data, CUZD1 represents a marker with better sensitivity and specificity than CA19.9. Combination of both CA 19.9 and CUZD1 results in 100% sensitivity but specificity drops slightly from 70% with CA 19.9 alone, to 65%.

### Example 4

In the previous dataset (Example 3), CA19-9 and CUZD-1 performed very similarly (slightly better for CA19-9) for the discrimination between benign and cancer patients. Next, it was decided to test the performance of these two markers in a bigger dataset which consisted of 50 normal, 50 benign (chronic pancreatitis, pancreatic cyst, PD dilation) and 50 cancer (of unknown stage) serum samples.

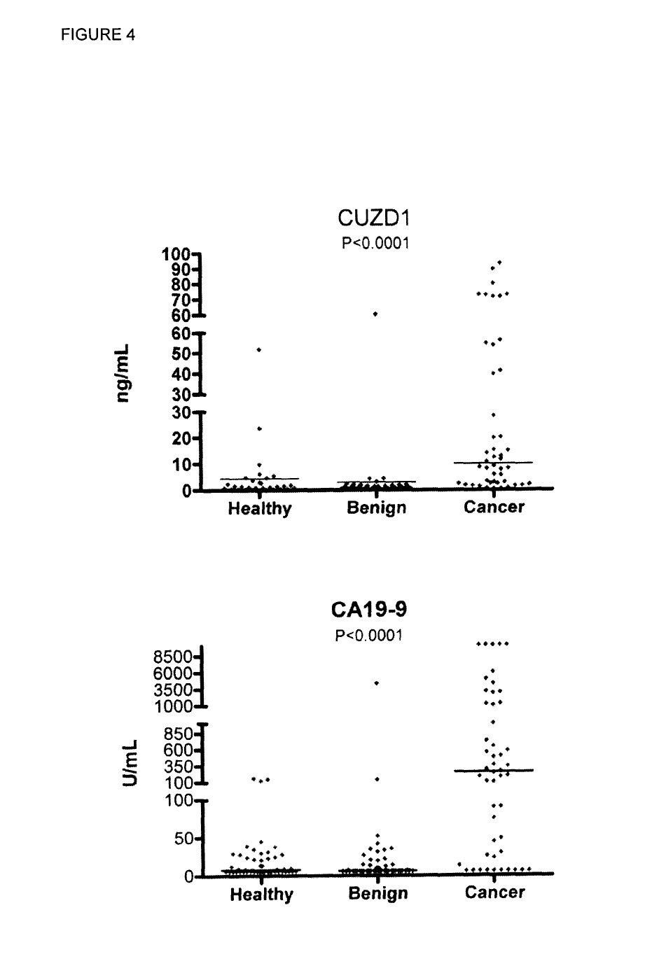

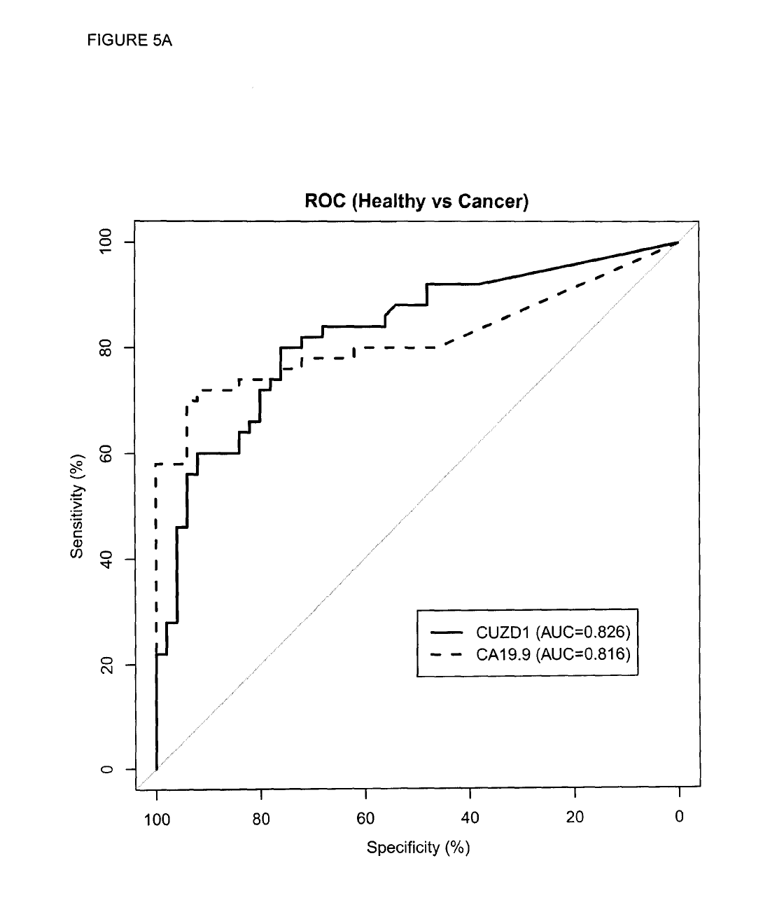

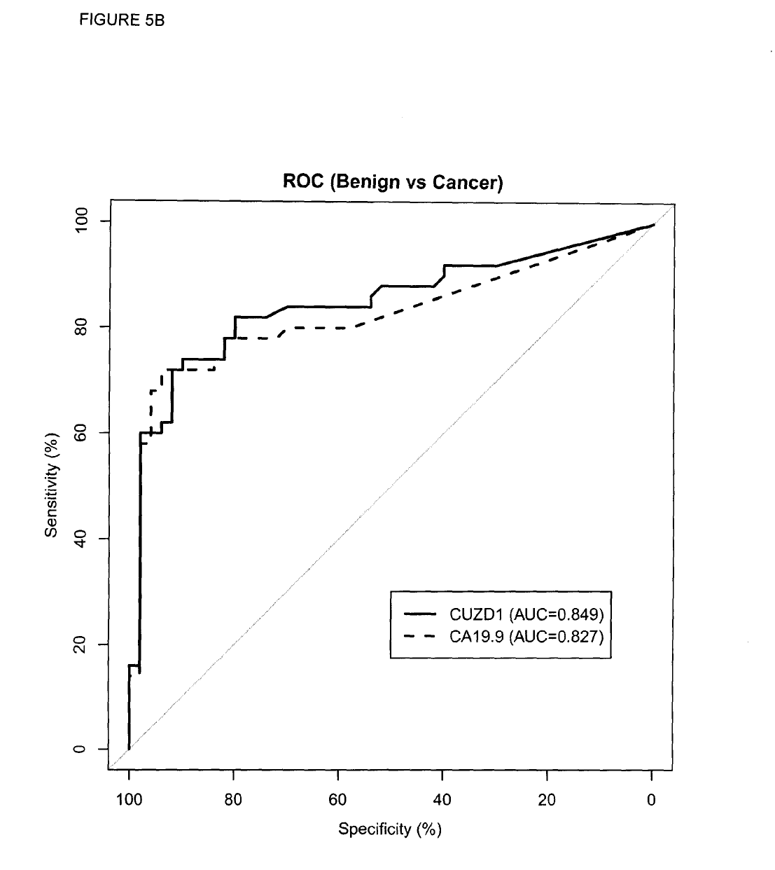

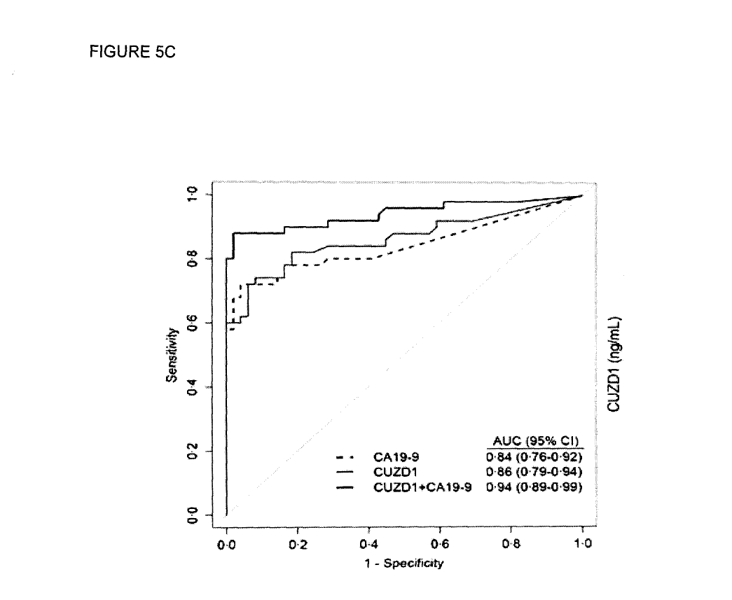

The scatter plot analysis for CUZD1 and CA19-9 (FIG. 4.) and the ROC curve analyses for CUZD1 and CA19-9—Normal Vs Cancer (FIG. 5A) and Benign Vs Cancer (FIG. 5B) demonstrate that CUZD1 out-performed CA19-9 in discriminating between benign and cancer patients. Interestingly, when the results of CA19-9 were examined, it was found that 14 out of the 50 cancer patients were negative for CA19-9 (less than 37 IU/L). However, among these, 8 were positive for CUZD-1 (more than 5 ng/ml) and another 3 were positive for LAMC2 (more than 150 ng/ml). The ROC curve analysis combining both CUZD1 and CA19-9 in Benign Vs PDAC shows that the combining these two markers out-performs CA19-9 and CUZD1 alone in discriminating between benign and cancer patients. FIG. 5C depicts the diagnostic performance of CA19-9 and CUZD1 in the dataset which consisted of 50 benign and 50 cancer (mixed stage) serum samples. The two markers displayed a similar potency in discriminating benign from neoplastic cases. Interestingly, there was a significant complementarity of the two markers.

### Example 5

### Discovery of LAMC2 and DSG2 Through Differential Tissue Proteomic Analysis of Pancreatic Adenocarcinoma (Benign Vs. Malignant)

Differential label-free semi-quantitative proteomining of pancreatic adenocarcinoma (PDAC) tissues and their adjacent benign tissues is a convenient approach for biomarker discovery. Herein, it is performed offline multi-dimensional chromatography/Orbitrap® mass spectrometry proteomic analysis of four PDAC tissues and their closest benign tissues to identify 2190 non-redundant proteins. 16 potential candidates using a systematic scoring algorithm were segregated, based on pancreatic cancer-specific mRNA overexpression, identification in malignant ascitic fluid, PDAC-label free quantitative value and cellular localization.

The preliminary serological verification of the top four candidates, DSP, LAMC2, GP73 and DSG2 in 20 patients diagnosed with pancreatic cancer and 20 with benign pancreatic cyst showed a significant (p<0.05) elevation for LAMC2 and DSG2 in pancreatic cancer serum. To validate the initial findings, it was decided to test the performance of these two markers in a bigger dataset which consisted of 50 normal, 50 benign (chronic pancreatitis, pancreatic cyst, PD dilation) and 50 cancer (of unknown stage) serum samples. Based on these initial results we decided to not analyze DSG2 further.

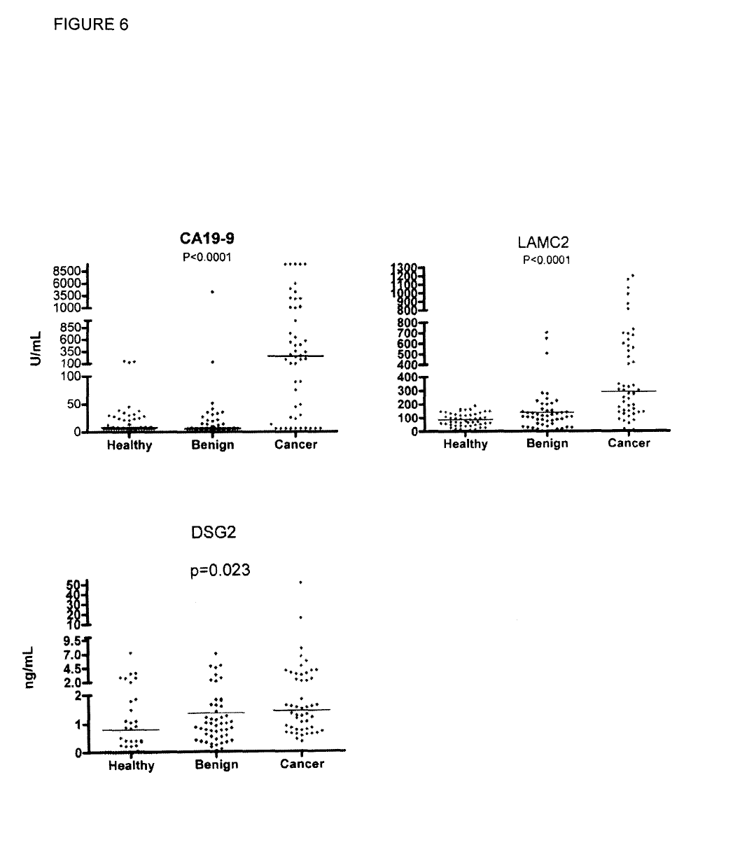

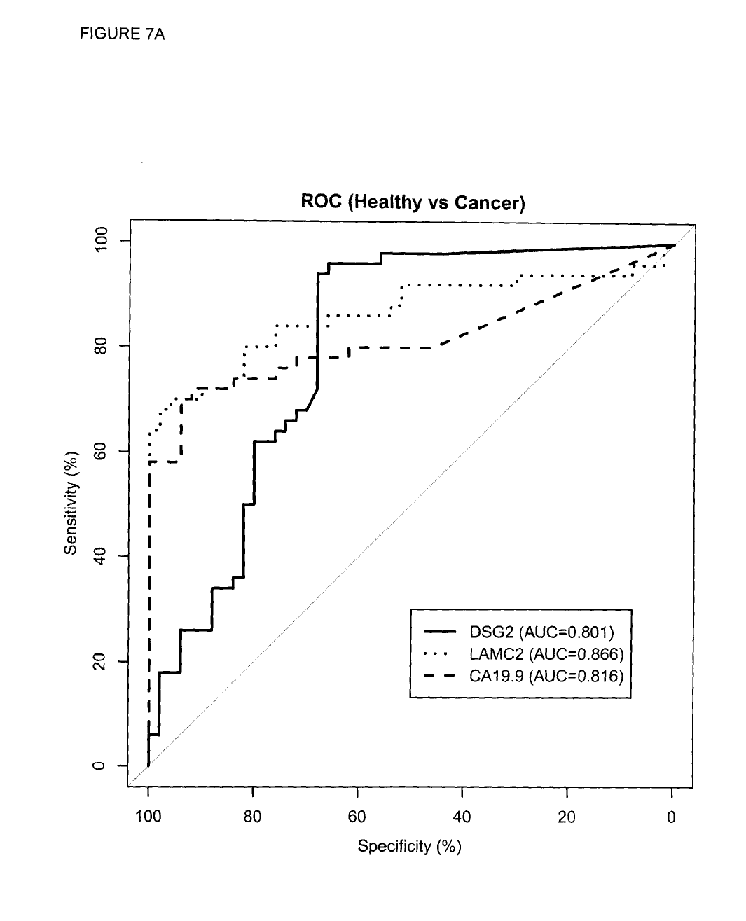

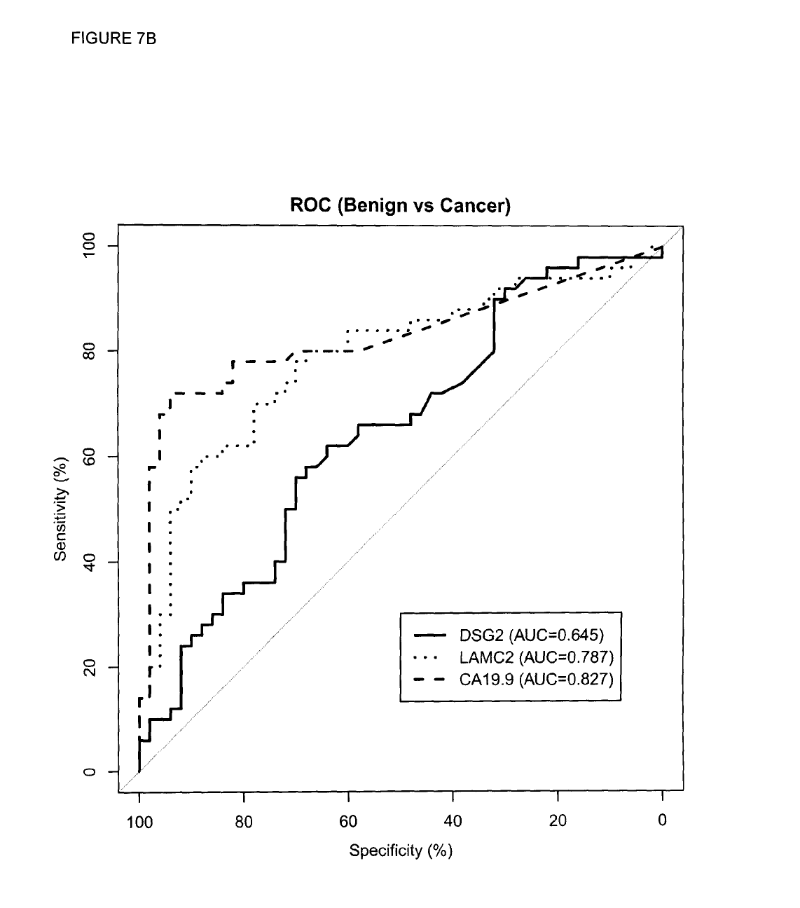

The scatter plot analyses for LAMC2, DSG2 and CA19-9 (FIG. 6) and the ROC curve analyses for these proteins (FIGS. 7A and 7B) demonstrate that LAMC2 outperformed CA19.9 (AUCs: 0.866 vs 0.816) in discriminating healthy individuals from cancer patients. On the contrary, CA19.9 displayed a higher discriminating efficiency between benign and cancer individuals, compared to both DSG2 and LAMC2 (AUCs; 0.827 for CA19-9, 0.787 for LAMC2, 0.645 for DSG2).

### Example 6

Given the results from the dataset of example 4, consisting of 50 benign and 50 cancer (mixed stage) serum samples, it was desired to investigate whether CUZD1 is also elevated in earlier stages of pancreatic cancer (stages I and II). To assess the performance of CUZD1 in early stages the serum levels of CUZD1 was measured in a second sample dataset which consisted of 50 normal, 50 benign, 50 cancer/stage II and 50 cancer/stage IV samples. CUZD1 was significantly elevated in the serum of pancreatic cancer patients even at stage II. Again a significant complementarity was seen when the two markers were used simultaneously.

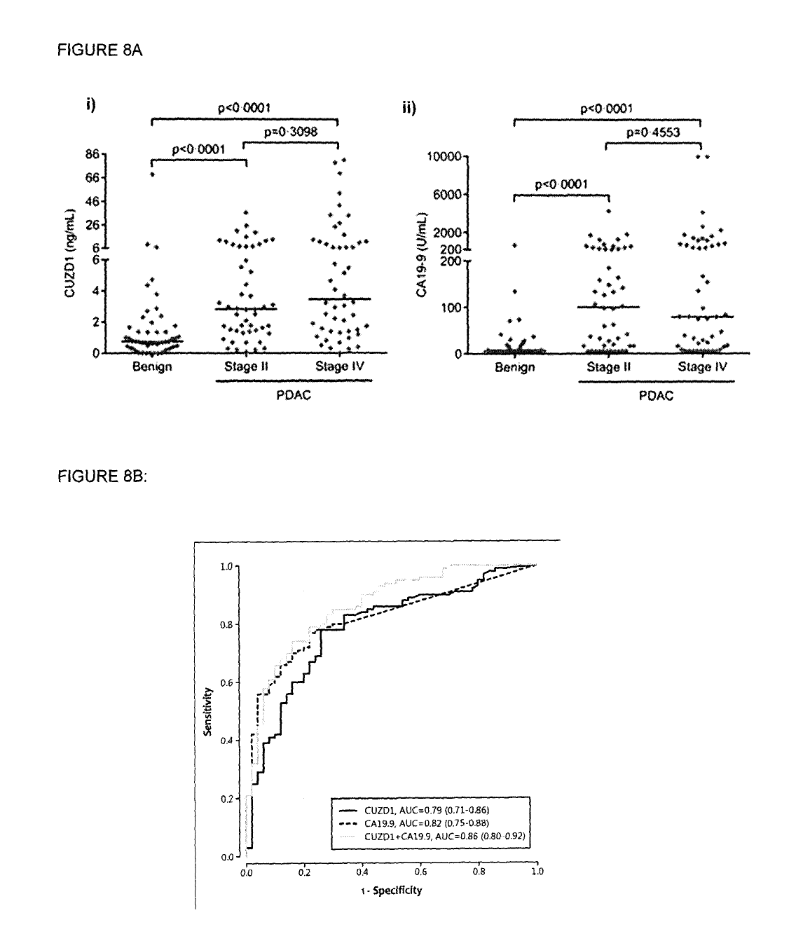

In the sample set, levels of CUZD1 were significantly elevated in patients with stage II and stage IV PDAC compared to patients with benign disease (stage II PDAC: median 2.83 ng/mL, IQR 1.43-7.42, P<0.0001; stage IV PDAC: median 3.46, IQR 1.40-11.48, P<0.0001), as were levels of CA19-9. ROC curve analysis (FIG. 8) showed similar performance of CUZD1 (AUC 0.79) and CA19-9 (AUC 0.82) in discriminating stage II and stage IV PDAC combined versus benign controls, with the combination of both markers increasing AUC to 0.85. CUZD1 was similarly informative between stage II (AUC=0.77) and stage IV disease (AUC=0.80). A greater proportion of patients with stage II and stage IV PDAC combined were positive for CA19-9 (63%) than CUZD1 (40%). The addition of CUZD1 increased the diagnostic sensitivity of CA19-9 from 63% to 74%, with a decreased specificity (four additional false positives, 88% to 80%). Furthermore, of the 37 CA19-9-negative patients with PDAC, 11 (30%) were positive for CUZD1.

### Example 7

### Blinded Study of American Cohort Comprising 85 Samples

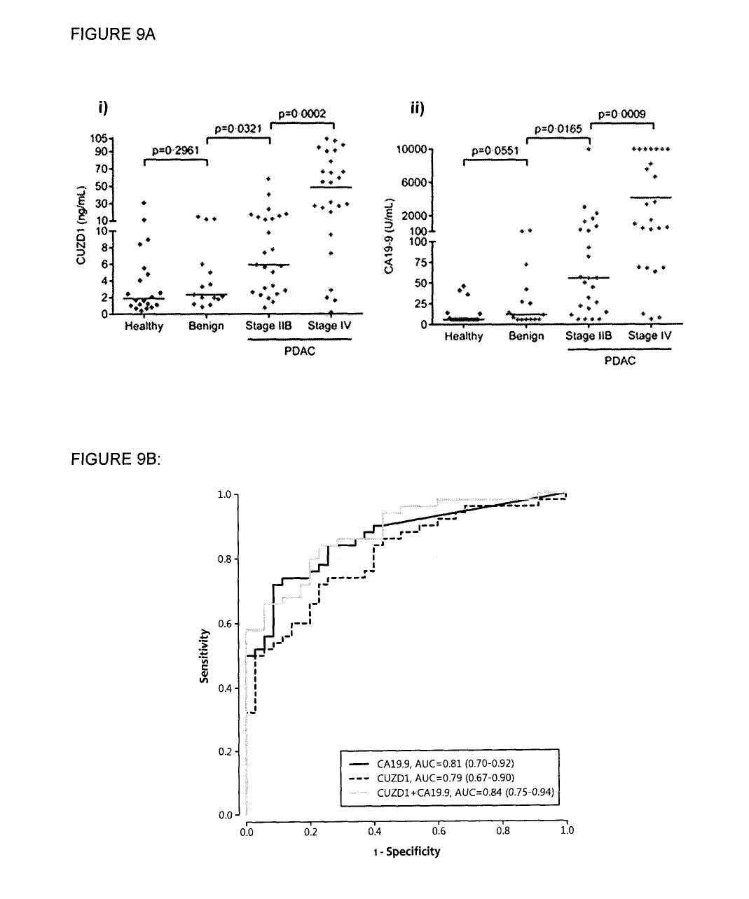

In the blinded sample set from Pittsburgh, Pa., USA, serum levels of CUZD1 were similar in patients with benign disease and healthy controls (P=0.2961). Levels of CUZD1 were significantly elevated in patients with stage IIB PDAC compared to patients with benign disease (stage IIB PDAC median 5.93 ng/mL, IQR 2.85-14.47; P=0.0321;). Levels of CUZD1 were also significantly elevated in patients with stage IV PDAC compared to those with stage IIB PDAC (stage IV PDAC median 54.40 ng/mL, IQR 20.33-79.02; P=0.0002;), FIG. 9A. This was similarly seen with CA19-9 (FIG. 9B). A slightly greater proportion of patients with stage IIB PDAC were positive for CUZD1 (64%) than CA19-9 (60%) and the addition of CUZD1 increased the diagnostic sensitivity of CA19-9 from 60% to 84% with some compromise in overall specificity (three additional false positives;). Furthermore, of the 10 CA19-9 negative patients with stage IIB PDAC, six (60%) were positive for CUZD1. ROC curve analysis (FIG. 9C) showed similar diagnostic value between CUZD1 (AUC 0.79) and CA19-9 (AUC 0.81) in discriminating between stage IIB and stage IV PDAC combined and benign controls, as well as complementarity between the two markers (AUC 0.85). The addition of CUZD1 increased the diagnostic sensitivity of CA19-9 in stage IIB and stage IV PDAC combined, from 74% to 90%, with some compromise in overall specificity (three additional false positives).

### Example 8

### Detection in Early Pancreatic Cancer

Pancreatic cancer (pancreatic ductal adenocarcinoma, PDAC) is the tenth most commonly diagnosed cancer but it ranks fourth in cancer-related deaths in North America^{101, 102}. In contrast to other major human malignancies (lung, breast, colon and prostate) which have shown notable reductions in mortality rate, attributed to earlier diagnosis and advancements in management and treatment, pancreatic cancer has had minimal improvement in patients' survival rate over the past 30 years¹⁰¹.

At the time of diagnosis, approximately 80% of patients demonstrate aggressive and metastatic tumours which are not suitable for surgical resection¹⁰³. The 5-year survival rate improves from 2% to 23% if the disease is diagnosed at its localized stage compared to a distant metastatic stage¹⁰⁴. Failure in therapeutic response in advanced disease is mainly attributed to the intense stromal effect in pancreatic cancer^{105, 106} and randomized clinical trials have suggested that adjuvant chemotherapy significantly enhance survival rates of patients who undergone surgical resection^{107, 108}, emphasizing the importance of early detection of the disease. The late presentation of disease-specific symptoms often leads to missed or delayed diagnosis of pancreatic cancer patients and hence decreased survival rates, emphasizing on the urgent clinical need to detect pancreatic cancer early before its progression to an advanced stage.

In terms of diagnosis, sensitive or specific screening tests for early detection of pancreatic cancer would be useful. Conventional imaging tools include computerized tomography (CT) scanning, magnetic resonance imaging (MRI), endoscopic ultrasonography (EUS), and endoscopic retrograde cholangiopancreatography (ERCP), which are powerful in tumour staging and confirming a suspected pancreatic mass^{109, 110}, are relatively costly, time-consuming and invasive. In the contrary, serum biomarkers have low cost and they are easily accessible, they remain to be an ideal way for early diagnosis¹¹¹. The current gold-standard serum biomarker CA19.9 is used in the clinic mainly for disease monitoring and prognosis^{102, 112, 113}. CA19.9 has limited sensitivity in pancreatic cancer detection due to its absence in Lewis^{a-b-} individuals (5-10% of Caucasian population) even in advanced disease stage, as well as it is barely detectable in early premalignant disease. CA19.9 is not a specific marker because of its elevation in other benign conditions and multiple cancer types. Taken together, it is critical to discover novel biomarkers to complement CA19.9 in order to improve both its sensitivity and specificity.

Using tissue proteomics¹¹⁶ and bioinformatics approaches¹¹⁷ CUB and zona pellucida-like domains 1 (CUZD1) and laminin, gamma C2 (LAMC2) respectively have been identified, which were recently discovered and validated as described above using three large independent sample sets with a total of 425 samples^{116, 119}.

Prior to our discovery and validation studies, there are very limited studies done on both of these markers in pancreatic cancer. In our validation results, CUZD1 and LAMC2 have demonstrated robust diagnostic performances in distinguishing pancreatic cancer from benign disease and they appear to have significant complementarity with CA19.9^{116, 119} A large blinded validation study of these markers using 400 patient plasma samples to evaluate their individual performances as well as their performance in a panel to complement CA19.9 in diagnosing early pancreatic cancer patients is described.

### Methods

### Study Population

Patients and control subjects were recruited on a consecutive basis from participating investigators in two major hospitals.

Subjects with a histologically confirmed or CT scan confirmed diagnosis of PDAC or with an abnormal abdominal imaging study (CT, MRI, MRCP and EUS) were eligible for the study. Control subjects with a clinical diagnosis of a pancreas, liver or intestinal condition, or being evaluated for non-pancreatic malignancies were included in the study. Subjects under the age of 18 years old and those without informed consent were excluded. Any patients with a prior history of any other malignancy except non-melanoma skin cancers for ten years were not included. Healthy controls were eligible volunteers without any of the pancreatic conditions or malignant diseases. A subset of patients was selected from the available subject pool based on desired characteristics (retrospective sample collection-prospective patient recruitment).

A total of 400 blinded plasma samples were obtained comprising of a training set (n=186) and an independent validation set (n=214). Overall, the 400 samples comprised of 20 healthy individuals, 130 benign condition patients, 51 stage IA, IB, 150 stage IIB and 49 stage IV pancreatic cancer patients. Details about sample population are shown in (Table 11). All samples were collected prior to any treatment following informed consent with an Institutional Review Board approved protocol.

### Measurement of Markers in Blood Samples

Blood was collected in ACD (anticoagulant) vacutainer tubes and plasma samples were processed within 24 hours of blood draw. Blood samples were centrifuged at room temperature for 10 minutes (at 1000×g) to pellet the cells. Right after the centrifugation, the plasma samples were aliquoted into 1 mL cryotubes stored in −80° C. until analysis.

Using commercially available sandwich enzyme-linked immunosorbent assays (ELISA) kits for, CUZD1 and LAMC2 purchased from USCN Life Sciences (Missouri City, Tex., USA), the levels of these proteins were measured in duplicates according to the manufacturer's protocols. CA19.9 levels were measured using the Abbott Architect XR CA19.9 ELISA immunoassay.

Prior to all validation assays, CUZD1 and LAMC2 ELISAs were first tested to optimize the analytical performances, to select appropriate internal controls (low, medium and high) and the dilution factor for each of the ELISA kits. Internal controls were used to assess the inter-plate variability.

Samples were diluted in assay buffer diluent as follows: 1 in 5 dilution for CUZD1 and 1 in 100 dilution for LAMC2. 100 uL of diluted sample was incubated in pre-coated ELISA 96-well plates along with standards for 2 hours in 37° C. After washing the strips, 100 uL of biotin-labeled polyclonal secondary antibody (detection reagent A) was added and incubated for another hour in 37° C. After washing, 100 uL of avidin-conjugated horseradish peroxidase (detection reagent B) was added and incubated for 30 minutes at 37° C. After a final washing step, 90 uL of tetramethylbenzidine (TMB) substrate was added to each well and incubated for approximately 10-15 minutes in the dark at 37° C. until the second lowest standard could be distinguished from the blank by a change of colour. 50 uL of stopping solution (sulphuric acid solution) was then added and the absorbance was measured using the Perkin-Elmer Envision 2103 Multilabel Reader at 450 nm wavelength standardized with a background absorbance at 540 nm.

The validation study was conducted according to the “Standards for the reporting of diagnostic accuracy studies (STARD) initiative”¹²⁰ (Table 15). Table 15 depicts an overall summary of the performance of CUZD1 and LAMC2 in comparison to CA19-9 in healthy, benign and cancer population.

### Statistical Analysis

Comparisons of levels of markers between groups was performed using the Mann Whitney-Wilcoxon test. Mean level comparisons were performed using a t-test and/or an ANOVA test.

Discriminative ability of biomarkers was assessed by building receiver operating characteristic curves (ROC) for individual markers and combined predictors. The diagnostic value of the markers was evaluated based on area under the curve (AUC) calculations and evaluation sensitivity at predetermined specificity thresholds of 80% and 90%. Confidence intervals (95%) for areas under the curve and p-value for comparison between two correlated ROC curves were performed using the method described by DeLong¹³⁰. An optimized cutoff for each marker was obtained by minimizing the total prediction error, by the following formula: \sqrt{(1−sensitivity)²+(1−specificity)²}.

Multi-parametric models for combinations of markers were constructed by fitting logistic regression models using the marker concentrations as predictors. The estimated coefficients of the model were used to construct a combined score for each observation which was then used for the evaluation of the multi-parametric model. The resulting 3 linear models evaluated for diagnostic performance are: (1) CA19.9+11.84·CUZD1, (2) CA19.9+0.202·LAMC2, (3) CA19.9+12.41·CUZD1+0.14·LAMC2.

Statistical analysis in the training set was performed while being blinded to clinical annotations of the validation set. After multi-parametric prediction models were build based on the training set samples, clinical information for validation samples were unblinded and model prediction were evaluated. Hypothesis testing was two-tailed, and p-values of less than 0.05 were considered as significant. Statistical analysis was performed in the R environment (version 2.15.2) available from http://www.R-project.org. ROC curve analysis and comparisons between ROC curves was performed using the pROC package¹²¹.

### Results

### Assay Performance

Prior to all validation assays, CUZD1 and LAMC2 ELISAs were first tested to optimize the analytical performances, to select appropriate internal controls (low, medium and high) and the dilution factor for each of the ELISA kits. Inter-plate assay imprecision was assessed across the 12 plates used for each marker using three internal controls (low, medium and high) (table 12). The coefficient of variation (CV) was calculated for each marker (Table 12). Overall, CUZD1 and LAMC2 assays demonstrated acceptable reproducibility across 12 plates, with <20% CVs in all three internal controls. As an additional quality control step, all samples were analyzed in duplicate to assess the intra-plate variations. The mean and median CV amongst duplicates samples ranged from 5% to 12% for all markers, which is indicative of good intra-plate performance of the assays.

All samples (n=400) were analyzed using ELISA assays on the same day for each candidate. Researchers I.P. and A.C. performed this step while being blinded to the clinical information of each sample.

### Performances of Markers in the Training and Validation Sets:

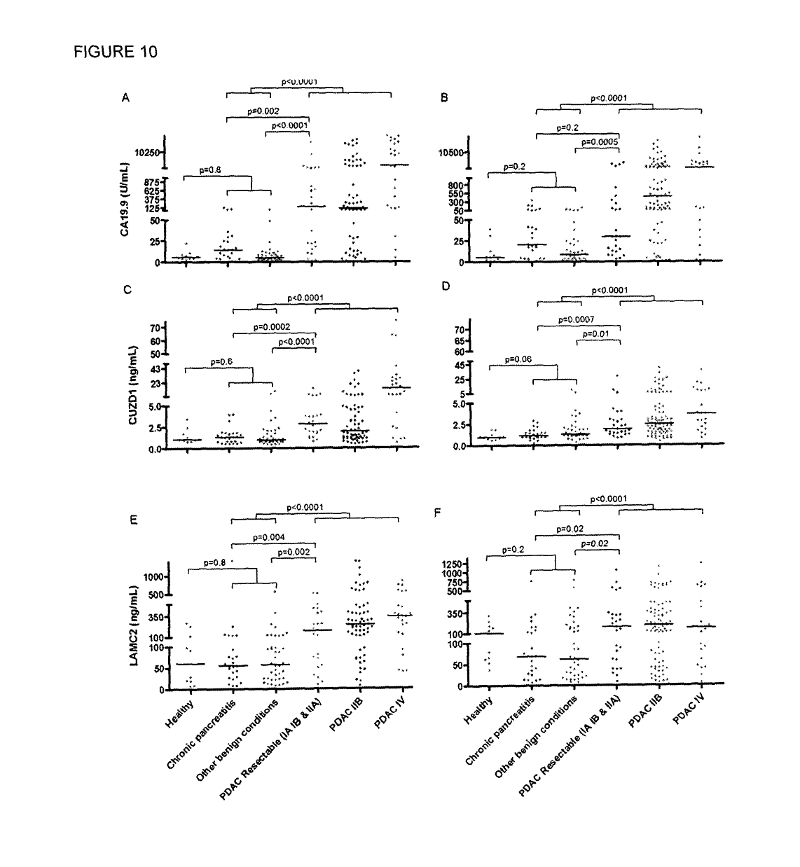

As individual markers, the performances of the candidates were compared to CA19.9 in discriminating benign patients versus PDAC patients in both training and validation cohorts (FIGS. 10 and 11A). CA19.9 concentrations were significantly higher in all patients with PDAC than all benign controls (median, mean, IQR; p<0.0001) in both training and validation cohorts (FIG. 10). CA19.9 was significantly elevated in resectable PDAC patients (stages IA, IB and IIA) compared to patients with chronic pancreatitis and other benign conditions (p<0.05) in the test cohort, but not in the validation cohort (FIG. 10). CUZD1 and LAMC2 demonstrated similar or better diagnostic ability than CA19.9 CUZD1 and LAMC2 concentrations were significantly increased in all PDAC cases compared to all benign controls in both training and validation cohorts (p<0.0001). Notably, CUZD1 and LAMC2 levels significantly differentiated early resectable PDAC patients (stages IA, IB and HA) from patients with chronic pancreatitis and other benign conditions (p<0.05) (FIG. 10). To compare individual markers, cutoffs were chosen based on the shortest distance of the ROC curve to the top-left corner. ROC curve showed the optimum diagnostic cutoff for CA19.9 was 20.3 U/mL, (area under the curve AUC 0.85, 95% CI 0.80-0.91, sensitivity 77.5%, specificity 83.1%; (FIG. 11A, Table 13). The optimum cutoff for CUZD1 was 1.8 ng/mL (AUC 0.77, 95% CI 0.71-0.84, sensitivity 64.9%, specificity 78.5%) and for LAMC2 was 123.2 ng/mL (AUC 0.81, 95% CI 0.75-0.88, sensitivity 70.3%, specificity 87.7%). Individually, CA19.9 had the greatest AUC in training and validation cohorts (FIG. 11A, Table 13). However, 22 out of 130 patients (approximately 17%) with benign disease were false positives with elevated CA19.9 levels (>37 IU/mL), limiting the specificity of CA19.9. CUZD1 appeared to have a higher specificity, whereas LAMC2 appeared to be a more sensitive candidate.

CA19.9 is not a reliable biomarker test in detecting early stage pancreatic cancer patients. The diagnostic ability CUZD1 and LAMC2 in complementing CA19.9 in early stages of pancreatic cancer patients (stages IA, IB and IIA), at which point the tumours are still generally resectable. Given that chronic pancreatitis often shows elevated level of CA19.9, CA19.9 lacks specificity in differentiating inflammatory from malignant masses, resulting in important therapeutic implications such as unnecessary surgery and undetected pancreatic malignancy. Therefore, the differential diagnostic accuracy of CUZD1 and LAMC2 was also assessed in chronic pancreatitis versus early PDAC patients.

Multi-parametric modeling for the combination of CA19.9, CUZD1 and LAMC2 as a two or three markers panel was constructed based on the training set and applied to the blinded validation set. ROC curves showed the performances of three models established in the training and validated sets respectively (FIG. 11C). Both performances of CA19.9 alone and the three models dropped in the validation set when compared to the training set. This may be resulted from different sample distribution in the two sets. Nevertheless, three models including CA19.9+CUZD1, CA19.9+LAMC2 and CA19.9+CUZD1+LAMC2 were found to significantly improve the AUC of CA19.9 alone in distinguishing all PDAC cases from all benign controls in training cohort (FIG. 11C, Table 13). Complementarity of CA19.9 with CUZD1 and LAMC2 was also demonstrated in distinguishing early resectable PDAC from chronic pancreatitis and other benign conditions compared to CA19.9 alone, with significant increase in the AUC from 0.69 to 0.79 and 0.59 to 0.73 respectively in the validation cohort (Table 13).

Performances of Candidates in PDAC Patients with CA19.9 Values Below 37 IU/mL

At its clinical cutoff value of 37 IU/mL, for diagnosing positive pancreatic cancer patients, CA19.9 has a reported sensitivity of 79-81% and specificity of 82-90%². Consequently, many PDAC cases are missed by CA19.9. The levels of CUZD1 and LAMC2 specifically in PDAC cases that had CA19.9 level <37 IU/mL were evaluated. Out of 250 PDAC cases in both training and validation sets, 75 PDAC cases (approximately 30%) had CA19.9 levels <37 IU/mL. In CA19.9-negative PDAC patients, CUZD1 and LAMC2 retained significant diagnostic ability to capture differentiate PDAC from benign conditions in both training and validation cohorts (p<0.05; Table 14). Notably, both CUZD1 and LAMC2 also had significant differential diagnosis of resectable CA19.9 negative PDAC patients (Table 14), demonstrating potential for complementarity for CA19.9.

The levels of CUZD1 and LAMC2 were evaluated specifically in PDAC cases that had CA19.9 level <37 IU/mL. Out of 250 PDAC cases in both training and validation sets, 75 PDAC cases (approximately 30%) had CA19.9 levels <37 IU/mL. In CA19.9-negative PDAC patients, CUZD1 and LAMC2 retained significant diagnostic ability to capture differentiate PDAC from benign conditions in both training and validation cohorts (p<0.05). Notably, both CUZD1 and LAMC2 also had significant differential diagnosis of resectable CA19.9 negative PDAC patients, demonstrating potential for complementarity for CA19.9.

### Discussion

A plethora of high-throughput discovery studies result in generation of thousands of potential diagnostic candidates, however, subsequent verification and validation studies are lacking in the biomarker field¹²². As a result, true biomarkers remained masked¹²³. To the best of our knowledge, there is currently no marker that can substitute CA19.9 in the clinic. CA19.9 is elevated in benign conditions and cancer types and can be undetectable in early resectable PDAC patients.

The present study is an extensive blinded validation and examines the diagnostic ability of CUZD1 and LAMC2 in complementing CA19.9 for example for detecting early stage PDAC patients, as well as differentiating between patients with benign conditions and PDAC patients. To avoid possible biases, we conducted our validation study according to the “Standards for the reporting of diagnostic accuracy studies (STARD) initiative”¹²⁰ (Table 15)3 CUZD1 and LAMC2 showed consistent and robust diagnostic performance throughout validation studies described in other Examples (n=425 samples)^{116, 119} and retained good diagnostic performances in the current 400 blinded sample set. CUZD1 and LAMC2 demonstrated strong diagnostic ability individually, they retained diagnostic accuracy in CA19.9 negative PDAC cases, and multi-parametric models demonstrated remarkable complementarity of CUZD1 and LAMC2 with CA19.9, especially in the detection of early stage PDAC (stages IA, IB, IIA and IIB) from benign conditions.

Recent research has suggested that it takes up to a decade before the initial tumour acquires metastatic ability, offering a long window of opportunity for early detection of pancreatic cancer^{124, 125}. Considering that no single marker possesses sufficient sensitivity and specificity for early diagnosis of pancreatic cancer, research interest has been shifted into the development of biomarker panels^{111, 126, 127}. A biomarker panel consisting of CA19.9, CUZD1 and LAMC2 can achieve better diagnostic performance in detecting PDAC patients than CA19.9 alone. This improvement is most notable at early disease stages when the disease may be treatable.

### Example 9

### CUZD1 and LAMC2 as a Monitoring Marker for Pancreatic Cancer

Monitoring pancreatic cancer patients is challenging. The only currently used marker is (CA19-9). Notably, almost 10% of the general population is genetically negative to CA19-9. Therefore, there is a need to identify novel markers that can complement CA19-9 as monitoring markers of the disease. Based on the data disclosed herein with CUZD1 and LAMC2, both marker could also be used as a monitoring marker for pancreatic cancer. Serum and tumour samples are currently being collecting from patients prior to surgery and/or during cycles of post-surgery chemotherapeutic treatments. Samples will be assessed for CUZD1 and compared to earlier and later obtained samples and correlated with disease progression. Prospective collection of serum from pancreatic cancer patients will follow.

### Example 10

Five highly colon-specific proteins were identified in the bioinformatics strategy to identify candidate biomarkers for colon cancer. In particular, the proteins: CLCA1 (HGNC_{—}2015, Entrez Gene_{—}1179, OMIM_{—}603906), GPA33 (HGNC_{—}4445, Entrez Gene_{—}10223 OMIM_{—}602171), LEFTY1 (HGNC_{—}6552, Entrez Gene_{—}10637, OMIM_{—}603037), ZG16 (Entrez_{—}16p11.2, HGNC_{—}16p11.2) and CEACAM7 (HGNC_{—}18191, Entrez Gene_{—}10872, Ensembl_ENSG000000073067, UniProtKB_Q140023) seem to fulfill the identified criteria that could characterize a promising biomarker candidate. Their expression is highly restricted to the colon, they are secreted or membrane-bound proteins and they have never been tested before as colon cancer serum markers. Serum samples are being collected from colon cancer patients in order to obtain an assessment of their performance in diagnosing colon cancer. Based on our results in-house immunoassays (ELISAs) will be made.

### Tables

TABLE 1
List of cell lines and relevant biological fluids of previously
characterized in-house proteomes

| | Tissue | Reference | |
|---|---|---|---|
| Conditioned | | | |
| Media of | | | |
| Cancer Cell | | | |
| Lines | | | |
| Colo320HSR | Colon | [Karagiannis G et al., unpublished] | |
| HCT116 | Colon | [Karagiannis G et al., unpublished] | |
| HT29 | Colon | [Karagiannis G et al., unpublished] | |
| LoVo | Colon | [Karagiannis G et al., unpublished] | |
| LS174T | Colon | [Karagiannis G et al., unpublished] | |
| LS180 | Colon | [Karagiannis G et al., unpublished] | |
| RKO | Colon | [Karagiannis G et al., unpublished] | |
| SW1116 | Colon | [Karagiannis G et al., unpublished] | |
| SW480 | Colon | [Karagiannis G et al., unpublished] | |
| SW620 | Colon | [Karagiannis G et al., unpublished] | |
| WiDr | Colon | [Karagiannis G et al., unpublished] | |
| H1688 | Lung | [27] | |
| H23 | Lung | [27] | |
| H460 | Lung | [27] | |
| H520 | Lung | [27] | |
| BxPc3 | Pancreas | [33] | |
| CAPAN1 | Pancreas | [33] | |
| CFPAC1 | Pancreas | [33] | |
| MIA-PaCa2 | Pancreas | [33] | |
| PANC1 | Pancreas | [33] | |
| SU.86.86 | Pancreas | [33] | |
| 22Rv1 | Prostate | [28] | |
| DU-145 | Prostate | [Saraon P, Diamandis EP; unpublished] | |
| LNCaP | Prostate | [28] | |
| LNCaP-SF | Prostate | [Saraon P, Diamandis EP; unpublished] | |
| PC3 | Prostate | [28] | |
| PPC-1 | Prostate | [Saraon P, Diamandis EP; unpublished] | |
| VCaP | Prostate | [Saraon P, Diamandis EP; unpublished] | |
| Conditioned | | | |
| Media of Near | | | |
| Normal Cell | | | |
| Lines | | | |
| RWPE | Prostate | [Saraon P, Diamandis EP; unpublished] | |
| HPDE | Pancreas | [33] | |
| Relevant | | | |
| Biological Fluid | Tissue | Condition | Reference |
| Ascites | Pancreas | | [32] |
| Tissue | Pancreas | Normal, Cancer | [Kosanam H, |
| | | | Diamandis EP; |
| | | | unpublished] |
| Seminal Plasma | | | [25] |
| Periotenal Fluid | | Non-malignant | [26] |
| Juice | Pancreas | | [33] |

TABLE 2
Total number of proteins identified from
mining gene and protein databases

| | Tissue | | | |
|---|---|---|---|---|
| | Colon | Lung | Pancreas | Prostate |
| Total Unique Proteins^{a} | 976 | 679 | 1059 | 623 |
| [in ≧2 databases] | [32] | [36] | [81] | [48] |
| Number of Proteins | | | | |
| Identified in . . . | | | | |
| 1 Database | 944 | 643 | 968 | 575 |
| 2 Databases | 23 | 30 | 46 | 32 |
| 3 Databases | 7 | 5 | 23 | 11 |
| 4 Databases | 1 | 1 | 9 | 4 |
| 5 Databases | 1 | | 3 | 1 |
| Number [%] of Secreted or Shed | 26 | 25 | 58 | 34 |
| Proteins in ≧2 Databases^{b} | [81%] | [69%] | [72%] | [71%] |

^{a}All proteins identified in ≧1 database; the number of total proteins identified with ≧2 databases is enclosed in brackets
^{b}Pertains to proteins identified using a Secretome Algorithm

TABLE 3
The number of proteins identified in each tissue, by each database.

| | Database | | | | | |
|---|---|---|---|---|---|---|
| | C-It | TiGER | UniGene | BioGPS | VeryGene | HPA |
| Colon | unavail- | 199 | 27 | 21 | 23 | 750 |
| | able | | | | | |
| Lung | 86 | 130 | 3 | 43 | 78 | 382 |
| Pancreas | 52 | 180 | 38 | 32 | 200 | 678 |
| Prostate | 116 | 127 | 16 | 31 | 64 | 339 |
| Total | 254 | 636 | 84 | 127 | 365 | 2149 |

TABLE 4
Forty eight proteins identified as tissue-specific, strongly expressed,
and secreted or shed in colon, lung, pancreas, or prostate tissue^{a}

| | | | | | | | | | Previously studied as a |
|---|---|---|---|---|---|---|---|---|---|
| | | | | | | | | | [tissue] cancer or |
| | | | | | | | | Very | benign disease |
| Tissue | | Accession | BioGPS | C-It | HPA | TiGER | UniGene | Gene | serum biomarker |
| Gene | Protein Name | Number | (12, 13) | (9) | (16) | (10) | (11) | (15) | (reference shown) |

Colon

| | | | | | | |
|---|---|---|---|---|---|---|
| CEACAM7 | Carcinoembryonic | IPI00028270 | ✓ | | ✓ | |
| | antigen-related | | | | | |
| | cell adhesion | | | | | |
| | molecule 7 | | | | | |
| CLCA1 | Chloride channel | IPI00014625 | ✓ | | ✓ | ✓ |
| | accessory 1 | | | | | |
| GPA33 | Glycoprotein A33 | IPI00293853 | | ✓ | ✓ | |
| | (transmembrane) | | | | | |
| LEFTY1 | Left-right | IPI00604473 | ✓ | | ✓ | |
| | determination | | | | | |
| | factor 1 | | | | | |
| ZG16 | Zymogen granule | IPI00029647 | ✓ | | ✓ | |
| | protein 16 | | | | | |
| | homolog (rat) | | | | | |

Lung

| | | | | | | | | |
|---|---|---|---|---|---|---|---|---|
| IRX5 | Iroquois | IPI00456865 | ✓ | ✓ | | ✓ | | |
| | homeobox 5 | | | | | | | |
| LAMP3 | Lysosomal- | IPI00004307 | | | | ✓ | ✓ | |
| | associated | | | | | | | |
| | membrane | | | | | | | |
| | protein 3 | | | | | | | |
| MFAP4 | Microfibrillar- | IPI00022792 | ✓ | ✓ | | | | |
| | associated | | | | | | | |
| | protein 4 | | | | | | | |
| SCGB1A1 | Secretoglobin, | IPI00006705 | ✓ | | | ✓ | ✓ | |
| | family 1A, | | | | | | | |
| | member 1 | | | | | | | |
| | (uteroglobin) | | | | | | | |
| SFTPA2 | Surfactant | IPI00293120 | ✓ | | | | ✓ | [54-56] |
| | protein A2 | | | | | | | |
| SFTPB | Surfactant | IPI00296083 | ✓ | | | ✓ | ✓ | [55] |
| | protein B | | | | | | | |
| SFTPC | Surfactant | IPI00006707 | | | ✓ | ✓ | | |
| | protein C | | | | | | | |
| SFTPD | Surfactant | IPI00291878 | ✓ | | | ✓ | ✓ | [56] |
| | protein D | | | | | | | |
| TMEM100 | Transmembrane | IPI00305416 | ✓ | ✓ | | | ✓ | |
| | protein 100 | | | | | | | |

Pancreas

| | | | | | | | | | |
|---|---|---|---|---|---|---|---|---|---|
| AQP8 | Aquaporin 8 | IPI00395685 | ✓ | | | | | ✓ | |
| CEL | Carboxyl ester | IPI00099670 | ✓ | | | ✓ | | ✓ | [60] |
| | lipase (bile salt- | | | | | | | | |
| | stimulated lipase) | | | | | | | | |
| CELA2A | Chymotrypsin- | IPI00829925 | | | | | ✓ | ✓ | [61] |
| | like elastase | | | | | | | | |
| | family, member | | | | | | | | |
| | 2A | | | | | | | | |
| CELA2B | Chymotrypsin- | IPI00027723 | ✓ | ✓ | | | ✓ | ✓ | |
| | like elastase | | | | | | | | |
| | family, member | | | | | | | | |
| | 2B | | | | | | | | |
| CELA3B | Chymotrypsin- | IPI00643846 | ✓ | | | | ✓ | | |
| | like elastase | | | | | | | | |
| | family, member | | | | | | | | |
| | 3B | | | | | | | | |
| CPA1 | Carboxypeptidase | IPI00009823 | ✓ | | ✓ | ✓ | ✓ | ✓ | [62] |
| | A1 (pancreatic) | | | | | | | | |
| CPA2 | Carboxypeptidase | IPI00941312 | ✓ | | ✓ | ✓ | | ✓ | [62] |
| | A2 (pancreatic) | | | | | | | | |
| CPB1 | Carboxypeptidase | IPI00009826 | ✓ | | | ✓ | ✓ | ✓ | [63] |
| | B1 (tissue) | | | | | | | | |
| CTRB1 | Chymotrypsinogen | IPI00015133 | ✓ | | | ✓ | | | |
| | B1 | | | | | | | | |
| CTRB2 | Chymotrypsinogen | IPI00742763 | ✓ | ✓ | | | ✓ | | |
| | B2 | | | | | | | | |
| CTRC | Chymotrypsin C | IPI00018553 | ✓ | | | ✓ | | ✓ | |
| | (caldecrin) | | | | | | | | |
| CUZD1 | Cub and zona | IPI00249672 | | ✓ | | ✓ | | ✓ | |
| | pellucida-like | | | | | | | | |
| | domains 1 | | | | | | | | |
| GCG | Glucagon | IPI00744153 | | | | ✓ | ✓ | ✓ | |
| IAPP | Islet amyloid | IPI00023679 | | | | ✓ | ✓ | ✓ | |
| | polypeptide | | | | | | | | |
| INS | Insulin | IPI00001508 | | | ✓ | ✓ | | ✓ | |
| KLK1 | Kallikrein 1 | IPI00304808 | ✓ | | | ✓ | | ✓ | |
| PNLIP | Pancreatic lipase | IPI00027720 | ✓ | | | ✓ | ✓ | ✓ | [64] |
| PNLIPRP1 | Pancreatic | IPI00005923 | | ✓ | | ✓ | | ✓ | |
| | lipase-related | | | | | | | | |
| | protein 1 | | | | | | | | |
| PNLIPRP2 | Pancreatic | IPI00005924 | ✓ | | | ✓ | | ✓ | |
| | lipase-related | | | | | | | | |
| | protein 2 | | | | | | | | |
| PPY | Pancreatic | IPI00000982 | | | | ✓ | ✓ | ✓ | |
| | prohormone | | | | | | | | |
| PRSS1 | Protease, serine, | IPI00946754 | ✓ | | ✓ | ✓ | ✓ | ✓ | [65] |
| | 1 (trypsin 1) | | | | | | | | |
| PRSS3 | Protease, serine, 3 | IPI00015614 | | | | ✓ | | ✓ | |
| REG1B | Regenerating | IPI00916240 | | | | ✓ | ✓ | ✓ | |
| | islet-derived 1 | | | | | | | | |
| | beta | | | | | | | | |
| REG3G | Regenerating | IPI00394807 | | ✓ | | ✓ | | ✓ | |
| | islet-derived 3 | | | | | | | | |
| | gamma | | | | | | | | |
| SLC30A8 | Solute carrier | IPI00217394 | | | | ✓ | ✓ | ✓ | |
| | family 30 (zinc | | | | | | | | |
| | transporter), | | | | | | | | |
| | member 8 | | | | | | | | |
| SYCN | Syncollin | IPI00397717 | ✓ | ✓ | | ✓ | ✓ | ✓ | [33] |

Prostate

| | | | | | | | | |
|---|---|---|---|---|---|---|---|---|
| ACPP | Acid | IPI00396434 | ✓ | ✓ | ✓ | ✓ | ✓ | [66] |
| | phosphatase, | | | | | | | |
| | prostate | | | | | | | |
| FOLH1 | Folate hydrolase | IPI00028514 | ✓ | | ✓ | | | [67] |
| | (prostate-specific | | | | | | | |
| | membrane | | | | | | | |
| | antigen) 1 | | | | | | | |
| KLK2 | Kallikrein-related | IPI00022227 | ✓ | | ✓ | | | [68] |
| | peptidase 2 | | | | | | | |
| KLK3 | Kallikrein-related | IPI00010858 | ✓ | ✓ | ✓ | | | [66] |
| | peptidase 3 | | | | | | | |
| NPY | Neuropeptide Y | IPI00001506 | | | ✓ | | ✓ | |
| PSCA | Prostate stem | IPI00013446 | ✓ | | ✓ | | | |
| | cell antigen | | | | | | | |
| RLN1 | Relaxin 1 | IPI00025853 | ✓ | | ✓ | ✓ | ✓ | |
| SLC45A3 | Solute carrier | IPI00064353 | ✓ | ✓ | ✓ | | ✓ | |
| | family 45, | | | | | | | |
| | member 3 | | | | | | | |

^{a}Tissue-specific proteins as it applies to this table indicates protein expression was manually verified in BioGPS and/or HPA databases. For database full names see “Non-Standard Abbreviations”

TABLE 5
List of colon tissue-specific proteins which have
not been previously studied as serum cancer

| | | Proteome Identified In: | |
|---|---|---|---|
| | | CM Proteome from | |
| | | Colon Cancer Cell | |
| Gene | Protein Name | Lines | Non-Colon Proteome |
| CEACAM7 | Carcinoembryonic | | ✓ CM proteome from |
| | antigen-related cell | | Hep 3B [52], pancreatic |
| | adhesion molecule 7 | | juice proteome [33] |
| CLCA1 | Chloride channel | | ✓ Normal, Down |
| | accessory 1 | | syndrome amniotic fluid |
| | | | [22, 23] |
| GPA33 | Glycoprotein A33 | ✓ LS174T^{a}, | |
| | | LS180^{a}, Colo205 | |
| | | [52] | |
| LEFTY1 | Left-right determination | | |
| | factor 1 | | |
| ZG16 | Zymogen granule | | ✓ CM proteome from |
| | protein 16 homolog (rat) | | Hep 3B [52] |

^{a}CM (conditioned media) proteome of colon cancer cell lines [Karagiannis G et al., unpublished].

TABLE 6
List of lung tissue-specific proteins which have
not been previously studied as serum cancer

| | | Proteome Identified In: |
|---|---|---|
| Gene | Protein Name | Non-Lung Proteome |
| IRX5 | Iroquois homeobox 5 | |
| LAMP3 | Lysosomal-associated | |
| | membrane protein 3 | |
| MFAP4 | Microfibrillar-associated | ✓ Normal and cancer |
| | protein 4 | pancreas tissue^{a}, seminal |
| | | plasma proteome [25], |
| | | non-malignant peritoneal fluid |
| SCGB1A1 | Secretoglobin, family | ✓ [22, 23, 25, 26, 31-33] |
| | 1A, member 1 | |
| | (uteroglobin) | |
| TMEM100 | Transmembrane protein | |
| | 100 | |

^{a}Proteome of normal and cancer pancreas tissue [Kosanam H et al., unpublished].

TABLE 7
List of pancreas tissue-specific proteins which
have not been previously studied as serum cancer

| | | Proteome Identified In: | | | | |
|---|---|---|---|---|---|---|
| | | Pancreatic | | | | |
| | | Cancer | Pancreatic | | | |
| | | Ascites | Juice | | | Non- |
| | | Proteome | Proteome | Pancreas Tissue^{a} | | Pancreas |
| Gene | Protein Name | [32] | [33] | Normal | Cancer | Proteome |
| AQP8 | Aquaporin 8 | | | | | |
| CTRB1 | Chmyotrypsinogen B1 | ✓ | | | | ✓ Down |
| | | | | | | Syndrome |
| | | | | | | amniotic |
| | | | | | | fluid [22] |
| CTRB2 | Chmyotrypsinogen B2 | | ✓ | ✓ | | |
| CUZD1 | CUB and zona | | ✓ | ✓ | | |
| | pellucida-like domains 1 | | | | | |
| KLK1 | Kallikrein 1 | | ✓ | ✓ | ✓ | |
| PNLIPRP1 | Pancreatic lipase- | | ✓ | ✓ | | |
| | related protein 1 | | | | | |
| PNLIPRP2 | Pancreatic lipase- | | ✓ | ✓ | | ✓ CM |
| | related protein 2 | | | | | proteome |
| | | | | | | from Hep 3B |
| | | | | | | [52] |
| PRSS3 | Protease, serine, 3 | ✓ | ✓ | ✓ | ✓ | ✓ HCC-38^{b}, |
| | | | | | | HCC-1143^{b}, |
| | | | | | | Normal |
| | | | | | | amniotic |
| | | | | | | fluid [23] |
| REG3G | Regenerating islet- | | ✓ | | | ✓ Seminal |
| | derived 3 gamma | | | | | plasma |
| | | | | | | proteome |
| | | | | | | [25] |
| SLC30A8 | Solute carrier family | | | | | |
| | 30 (zinc transporter), | | | | | |
| | member 8 | | | | | |

^{a}Proteome of normal and cancer pancreas tissue [Kosanam H et al., unpublished].
^{b}CM Proteome of breast cancer cell lines [Pavlou M et al., unpublished].

TABLE 8
List of prostate-specific proteins which have not been previously
studied as serum cancer

| | | Proteome Identified In: | | |
|---|---|---|---|---|
| | | CM Proteome | Seminal | |
| | | from Prostate | Plasma | |
| | | Cancer Cell | Proteome | Non-Prostate |
| Gene | Protein Name | Lines | (20) | Proteome |
| NPY | Neuropeptide | ✓ VCaP^{a} | ✓ | |
| | Y | | | |
| PSCA | Prostate stem | ✓ PC3 [28] | ✓ | ✓ Normal and |
| | cell antigen | | | cancer pancreas |
| | | | | tissue^{b}, CM |
| | | | | proteome from |
| | | | | pancreatic |
| | | | | cancer |
| | | | | cell lines |
| | | | | SU.86.86, |
| | | | | CAPAN1 [33] |
| RLN1 | Relaxin 1 | | | |
| SLC45A3 | Solute carrier | | | |
| | family 45, | | | |
| | member 3 | | | |

^{a}CM proteome from prostate cancer cell line [Saraon P et al., unpublished].
^{b}Proteome of normal and cancer pancreas tissue [Kosanam H et al., unpublished].

TABLE 9
ELISA serum levels of CA19.9 and CUZD1 in 20 pancreatic cyst and 20
pancreatic cancer samples.

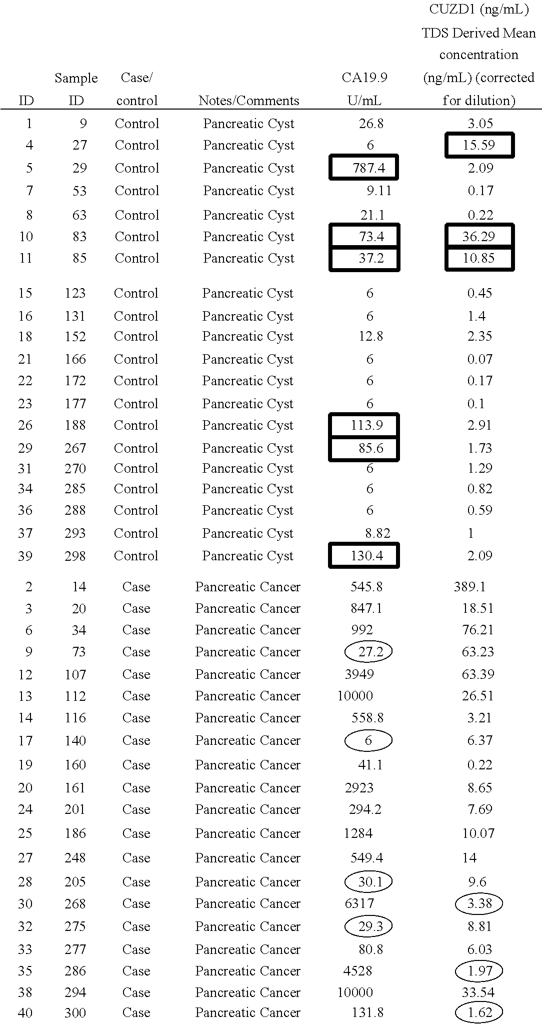

TABLE 10
Descriptive statistics of CA19.9 and CUZD1 (CI = 95% confidence interval)

| | | | | | p-value | | | |
|---|---|---|---|---|---|---|---|---|
| | Median | Median | Median | Mean | (Wilcoxon | | Lower | Upper |
| | Benign | Cancer | Ratio | Ratio | test) | AUC | CI | CI |
| CA19.9 | 8.97 | 554.1 | 61.81 | 31.7 | 0.000034 | 0.88 | 0.76 | 0.97 |
| CUZD1 | 1.35 | 9.21 | 6.84 | 9.04 | 0.00026 | 0.84 | 0.69 | 0.95 |

TABLE 11
Sample characteristics in training and validation sets.

| Sample | | | |
|---|---|---|---|
| characteristics | Training | Validation | Total |
| Healthy control | 10 | 10 | 20 |
| Acute pancreatitis | 6 | 23 | 29 |
| Chronic pancreatitis | 25 | 25 | 50 |
| CBD stones | 19 | 0 | 19 |
| Other benign | 15 | 17 | 32 |
| conditions | | | |
| PDAC, stage IA | 4 | 5 | 9 |
| PDAC, stage IB | 3 | 5 | 8 |
| PDAC, stage IIA | 17 | 17 | 34 |
| PDAC, stage IIB | 62 | 88 | 150 |
| PDAC, stage IV | 25 | 24 | 49 |
| Total | 186 | 214 | 400 |
| Number of | 84/101* | 110/104 | 194/205 |
| females/males | | | |
| Median (mean) age | 66.0 (63.0) | 64.0 (63.1) | 65.0 (63.1) |
| Smoking history | 35C/88NE/62P | 43C/74NE/70P | 78C/162NE/132P |
| | (1 unknown) | (2 unknown) | |
| Diabetic history | 53Y/131N | 25Y/189N | 78Y/320N |
| | (2 unknown) | | |

PDAC = pancreatic ductal adenocarcinoma;
Y = yes;
N = no;
C = current;
NE = never;
P = past
*One sample did not contain sex information

Samples characterized by Acute pancreatitis, Chronic pancreatitis, CBD stones and Other benign conditions are identified as being “Benign”; Samples characterized by PDAC, stage IA, IB, IIA are identified as being “Resectable”; Samples characterized by PDAC, stage IIB are identified as “Maybe resectable”; Samples characterized as PDAC, stage IV are identified as “Non-resectable”.

TABLE 12
a. % CV and mean of three internal controls for each protein
(intra-assay reproducibility). b. Mean and median of % CV
for duplicates in all samples for each protein.

| | | LAMC2 | CUZD1 |
|---|---|---|---|
| a. Internal | Low control | 9 (208.6) | 16 (0.4) |
| Controls | Medium control | 12 (6009.9) | 14 (1.9) |
| % CV (Mean) | High control | 13 (7053.5) | 16 (7.6) |
| b. Duplicates | Median | 6.62 | 5.31 |
| % CV | Mean | 10.07 | 7.85 |

Concentrations (ng/mL) prior to correcting for dilution factor are listed for all five candidates. Blank cells were not shown.

TABLE 13
Performances of CA19.9, CUZD1, LAMC2, two- and three- markers models in diagnosis of PDAC

| Test | | | Validation | | |
|---|---|---|---|---|---|
| AUC (95% CI) | Sensitivity | Specificity | AUC (95% CI) | Sensitivity | Specificity |

Benign vs all PDAC

| | | | | | | |
|---|---|---|---|---|---|---|
| CA19.9 | 0.85 (0.80-0.91) | 77.5% | 83.1% | 0.80 (0.74-0.86) | 79.1% | 63.1% |
| CUZD1 | 0.77 (0.71-0.84) | 64.9% | 78.5% | 0.76 (0.69-0.83) | 66.2% | 72.3% |
| LAMC2 | 0.81 (0.75-0.88) | 70.3% | 87.7% | 0.69 (0.62-0.77) * | 61.2% | 69.2% |
| CA19.9 + CUZD1 | 0.90 (0.86-0.95) * | 81.1% | 87.7% | 0.86 (0.82-0.91) ** | 81.3% | 70.8% |
| CA19.9 + LAMC2 | 0.91 (0.87-0.95) * | 82.9% | 89.2% | 0.83 (0.77-0.88) | 80.6% | 61.5% |
| CA19.9 + CUZD1 + LAMC2 | 0.93 (0.89-0.96) ** | 87.4% | 87.7% | 0.87 (0.82-0.92) ** | 86.3% | 64.6% |

Benign vs early PDAC (stage IA, IB & IIA)

| | | | | | | |
|---|---|---|---|---|---|---|
| CA19.9 | 0.82 (0.69-0.94) | 70.8% | 83.1% | 0.69 (0.57-0.82) | 59.3% | 63.1% |
| CUZD1 | 0.81 (0.72-0.91) | 75.0% | 78.5% | 0.72 (0.60-0.83) | 51.9% | 72.3% |
| LAMC2 | 0.73 (0.60-0.86) | 58.3% | 87.7% | 0.68 (0.56-0.80) | 59.3% | 69.2% |
| CA19.9 + CUZD1 | 0.91 (0.84-0.98) | 75.0% | 86.2% | 0.75 (0.63-0.86) | 59.3% | 70.8% |
| CA19.9 + LAMC2 | 0.85 (0.74-0.96) | 75.0% | 89.2% | 0.75 (0.64-0.86) | 74.1% | 61.5% |
| CA19.9 + CUZD1 + LAMC2 | 0.91 (0.83-0.99) | 79.2% | 86.2% | 0.79 (0.70-0.89) * | 74.1% | 64.6% |

CP vs early PDAC

| | | | | | | |
|---|---|---|---|---|---|---|
| CA19.9 | 0.76 (0.62-0.90) | 70.8% | 68.0% | 0.59 (0.44-0.75) | 59.3% | 48.0% |
| CUZD1 | 0.82 (0.70-0.94) | 75.0% | 84.0% | 0.78 (0.65-0.90) | 51.9% | 80.0% |
| LAMC2 | 0.74 (0.59-0.88) | 58.3% | 88.0% | 0.69 (0.54-0.83) | 59.3% | 72.0% |
| CA 19.9 + CUZD1 | 0.88 (0.79-0.98) * | 75.0% | 84.0% | 0.68 (0.54-0.83) * | 59.3% | 60.0% |
| CA19.9 + LAMC2 | 0.82 (0.69-0.95) | 70.8% | 88.0% | 0.70 (0.55-0.84) | 74.1% | 44.0% |
| CA19.9 + CUZD1 + LAMC2 | 0.89 (0.79-0.99) * | 75.0% | 84.0% | 0.73 (0.60-0.87) * | 74.1% | 52.0% |

PDAC = pancreatic ductal adenocarcinoma.
CP = chronic pancreatitis.
AUC = area under curve.
* p < 0.05,
** p < 0.005 in comparison to CA19.9.

TABLE 14
Performances of CUZD1, LAMC2 in diagnosis of CA19.9 negative PDAC patients

| CA19.9 | Test | | | Validation | | |
|---|---|---|---|---|---|---|
| negative | AUC (95% CI) | Sensitivity | Specificity | AUC (95% CI) | Sensitivity | Specificity |

Benign vs all PDAC

| | | | | | | |
|---|---|---|---|---|---|---|
| CUZD1 | 0.75 (0.65-0.85) ** | 61.8% | 76.7% | 0.76 (0.66-0.87) ** | 65.9% | 77.1% |
| LAMC2 | 0.76 (0.65-0.86) ** | 52.9% | 88.3% | 0.64 (0.53-0.76) * | 53.7% | 68.8% |

Benign vs early PDAC (stage IA, IB & IIA)

| | | | | | | |
|---|---|---|---|---|---|---|
| CUZD1 | 0.78 (0.63-0.93) ** | 63.6% | 75.0% | 0.73 (0.58-0.88) * | 53.3% | 77.1% |
| LAMC2 | 0.66 (0.47-0.85) | 36.4% | 86.7% | 0.70 (0.54-0.85) * | 66.7% | 68.8% |

PDAC = pancreatic ductal adenocarcinoma.
AUC = area under curve.
* p < 0.05,
** p < 0.005.

TABLE 15
Statistics of each marker in healthy, benign and cancer patients.

| Marker | Group | # | min | max | median | mean | IQR |
|---|---|---|---|---|---|---|---|
| CA19.9 | adenocarcinoma | 51 | 2 | 16244.99 | 36.672 | 1005.682 | 673.917 |
| | stage IA/IB/IIA | | | | | | |
| | adenocarcinoma | 150 | 2 | 17501.66 | 246.031 | 2226.131 | 2203.462 |
| | stage IIB | | | | | | |
| | adenocarcinoma | 49 | 2 | 19858.33 | 2370.807 | 5285.785 | 9257.63 |
| | stage IV | | | | | | |
| | benign condition | 130 | 2 | 354.973 | 8.461 | 23.214 | 19.417 |
| | healthy control | 20 | 2 | 39.356 | 5.714 | 9.465 | 5.512 |
| CUZD1 | adenocarcinoma | 51 | 0.82 | 29.055 | 2.295 | 3.546 | 2.292 |
| | stage IA/IB/IIA | | | | | | |
| | adenocarcinoma | 150 | 0.49 | 40 | 2.417 | 5.175 | 3.415 |
| | stage IIB | | | | | | |
| | adenocarcinoma | 49 | 0.635 | 74.65 | 6.655 | 15.782 | 18.36 |
| | stage IV | | | | | | |
| | benign condition | 130 | 0.28 | 11.265 | 1.23 | 1.688 | 0.994 |
| | healthy control | 20 | 0.655 | 3.475 | 1.075 | 1.295 | 0.572 |
| LAMC2 | adenocarcinoma | 51 | 9.568 | 1059.826 | 172.172 | 230.776 | 272.53 |
| | stage IA/IB/IIA | | | | | | |
| | adenocarcinoma | 150 | 7.196 | 1387.508 | 207.868 | 288.555 | 296.284 |
| | stage IIB | | | | | | |
| | adenocarcinoma | 49 | 6.492 | 1226.765 | 229.12 | 304.453 | 367.804 |
| | stage IV | | | | | | |
| | benign condition | 130 | 0 | 1402.762 | 59.905 | 114.086 | 92.738 |
| | healthy control | 20 | 7.127 | 315.591 | 85.848 | 111.314 | 137.562 |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications as well as sequences corresponding to the accession numbers listed in the Tables, are herein incorporated by reference in their entirety to the same extent as if each individual publication, patent, patent application or sequence was specifically and individually indicated to be incorporated by reference in its entirety. Specifically, the sequence associated with each accession number provided herein is incorporated by reference in its entirely.

### REFERENCES

1. Kulasingam V, Diamandis E P: Strategies for discovering novel cancer biomarkers through utilization of emerging technologies. Nat Clin Pract Oncol 2008, 5:588-599.
2. Diamandis E P: Cancer biomarkers: can we turn recent failures into success? J Natl Cancer Inst 2010,102:1462-1467.
3. Fletcher R H: Carcinoembryonic antigen. Ann Intern Med 1986, 104:66-73.
4. Duffy M J: CA 19-9 as a marker for gastrointestinal cancers: A review. Ann Clin Biochem 1998, 35:364-370.
5. Goonetilleke K S, Siriwardena A K: Systematic review of carbohydrate antigen (CA 19-9) as a biochemical marker in the diagnosis of pancreatic cancer. Eur J Surg Oncol 2007,33:266-270.
6. Schneider J: Tumor markers in detection of lung cancer. Adv Clin Chem 2006, 42:1-41.
7. Bostwick D G: Prostate-specific antigen. Current role in diagnostic pathology of prostate cancer. Am J Clin Pathol 1994, 102(4 Suppl 1):S31-7.
8. Barry M J: Screening for prostate cancer—the controversy that refuses to die. N Engl J Med 2009,360:1351-1354.
9. Gellert P, Jenniches K, Braun T, Uchida S: C-It: a knowledge database for tissue-enriched genes. Bioinformatics 2010, 26:2328-2333.
10. The C-It Database [http://c-it.mpi-bn.mpg.de].
11. Liu X, Yu X, Zack D J, Zhu H, Qian J: TiGER: a database for tissue-specific gene expression and regulation. BMC Bioinformatics 2008, 9:271.
12. The TiGER Database [http://bioinfo.wilmer.jhu.edu/tiger].
13. Pontius J U, Wagner L, Schuler G D: UniGene: a unified view of the transcriptome. In The NCBI Handbook. Edited by McEntyre J, Ostell J. Bethesda (MD): National Center for Biotechnology Information (US); 2002:21.1-21.11.
14. The UniGene Database [http://www.ncbi.nlm.nih.gov/unigene].
15. Wu C, Orozco C, Boyer J, Leglise M, Goodale J, Batalov S, Hodge C L, Haase J, Janes J, Huss J W 3rd, Su A I: BioGPS: an extensible and customizable portal for querying and organizing gene annotation resources. Genome Biol 2009, 10:R130.
16. Su A, Wiltshire T, Batalov S, Lapp H, Ching K, Block D, Zhang J, Soden R, Hayakawa M, Kreiman G, Cooke M, Walker J, Hogenesch J: A gene atlas of the mouse and human protein-encoding transcriptomes. Proc Natl Acad Sci USA 2004, 101:6062-6067.
17. The BioGPS Database [http://biogps.org].
18. Yang X, Ye Y, Wang G, Huang H, Yu D, Liang S: VeryGene: linking tissue-specific genes to diseases, drugs, and beyond for knowledge discovery. Physiol Genomics 2011, 43:457-460.
19. The VeryGene Database [http://www.verygene.com].
20. Uhlen M, Oksvold P, Fagerberg L, Lundberg E, Jonasson K, Forsberg M, Zwahlen M, Kampf C, Wester K, Hober S, Wernerus H, Björling L, Ponten F: Towards a knowledge-based Human Protein Atlas. Nat Biotechnol 2010, 28:1248-1250.
21. The Human Protein Atlas [http://proteinatlas.org].
22. Cho C K, Smith C R, Diamandis E P: Amniotic fluid proteome analysis from Down syndrome pregnancies for biomarker discovery. J Proteome Res 2010, 9:3574-3582.
23. Cho C K, Shan S J, Winsor E J, Diamandis E P: Proteomics analysis of human amniotic fluid. Mol Cell Proteomics 2007, 6:1406-1415.
24. Kuk C, Kulasingam V, Gunawardana C G, Smith C R, Batruch I, Diamandis E P: Mining the ovarian cancer ascites proteome for potential ovarian cancer biomarkers. Mol Cell Proteomics 2009, 8:661-669.
25. Batruch I, Lecker I, Kagedan D, Smith C R, Mullen B J, Grober E, Lo K C, Diamandis E P, Jarvi K A: Proteomic analysis of seminal plasma from normal volunteers and post-vasectomy patients identifies over 2000 proteins and candidate biomarkers of the urogenital system. J Proteome Res 2011, 10:941-953.
26. Gunawardana C G, Memari N, Diamandis E P: Identifying novel autoantibody signatures in ovarian cancer using high-density protein microarrays. Clin Biochem 2009, 42:426-429.
27. Planque C, Kulasingam V, Smith C R, Reckamp K, Goodglick L, Diamandis E P: Identification of five candidate lung cancer biomarkers by proteomics analysis of conditioned media of four lung cancer cell lines. Mol Cell Proteomics 2009, 8:2746-2758.
28. Sardana G, Jung K, Stephan C, Diamandis E P: Proteomic analysis of conditioned media from the PC3, LNCaP, and 22Rv1 prostate cancer cell lines: discovery and validation of candidate prostate cancer biomarkers. J Proteome Res 2008,7:3329-3338.
29. Gunawardana C G, Kuk C, Smith C R, Batruch I, Soosaipillai A, Diamandis E P: Comprehensive analysis of conditioned media from ovarian cancer cell lines identifies novel candidate markers of epithelial ovarian cancer. J Proteome Res 2009, 8:4705-4713.
30. Kulasingam V, Diamandis E P: Proteomics analysis of conditioned media from three breast cancer cell lines: a mine for biomarkers and therapeutic targets. Mol Cell Proteomics 2007, 6:1997-2011.
31. Pavlou M P, Kulasingam V, Sauter E R, Kliethermes B, Diamandis E P: Nipple aspirate fluid proteome of healthy females and patients with breast cancer. Clin Chem 2010, 56:848-855.
32. Kosanam H, Makawita S, Judd B, Newman A, Diamandis E P: Mining the malignant ascites proteome for pancreatic cancer biomarkers. Proteomics 2011, 11:4551-4558.
33. Makawita S, Smith C, Batruch I, Zheng Y, Rückert F, GrOtzmann R, Pilarsky C, Gallinger S, Diamandis E P: Integrated proteomic profiling of cell line conditioned media and pancreatic juice for the identification of pancreatic cancer biomarkers. Mol Cell Proteomics 2011, 10:M111.008599.
34. Nannini M, Pantaleo M A, Maleddu A, Astolfi A, Formica S, Biasco G: Gene expression profiling in colorectal cancer using microarray technologies: results and perspectives. Cancer Treat Rev 2009, 35:201-209.
35. Petty R D, Nicolson M C, Kerr K M, Collie-Duguid E, Murray G I: Gene expression profiling in non-small cell lung cancer: from molecular mechanisms to clinical application. Clin Cancer Res 2004, 10:3237-3248.
36. Cardoso J, Boer J, Morreau H, Fodde R: Expression and genomic profiling of colorectal cancer. Biochim Biophys Acta 2007, 1775:103-137.
37. Magnusson K, de Wit M, Brennan D J, Johnson L B, McGee S F, Lundberg E, Naicker K, Klinger R, Kampf C, Asplund A, Wester K, Gry M, Bjartell A, Gallagher W M, Rexhepaj E, Kilpinen S, Kallioniemi O P, Belt E, Goos J, Meijer G, Birgisson H, Glimelius B, Borrebaeck C A, Navani S, Uhlén M, O'Connor D P, Jirström K, Pontén F: SATB2 in combination with Cytokeratin 20 identifies over 95% of all colorectal carcinomas. Am J Surg Pathol 2011, 35:937-948.
38. Ehlen O, Nodin B, Rexhepaj E, Brandstedt J, Uhlen M, Alvarado-Kristensson M, Ponten F, Brennan D J, Jirstrom K: RBM3-regulated genes promote DNA integrity and affect clinical outcome in epithelial ovarian cancer. Transl Oncol 2011, 4:212-221.
39. Borgquist S, Djerbi S, Ponten F, Anagnostaki L, Goldman M, Gaber A, Manjer J, Landberg G, Jirstrom K: HMG-CoA reductase expression in breast cancer is associated with a less aggressive phenotype and influenced by anthropometric factors. Int J Cancer 2008, 123:1146-1153.
40. Borgquist S, Jögi A, Pontén F, Rydén L, Brennan D J, Jirström K: Prognostic impact of tumour-specific HMG-CoA reductase expression in primary breast cancer. Breast Cancer Res 2008, 10:R79.
41. Gaber A, Johansson M, Stenman U H, Hotakainen K, Ponten F, Glimelius B, Bjartell A, Jirstrom K, Birgisson H: High expression of tumour-associated trypsin inhibitor correlates with liver metastasis and poor prognosis in colorectal cancer. Br J Cancer 2009, 100:1540-1548.
42. Ghanipour A, Jirstrom K, Ponten F, Glimelius B, Pahlman L, Birgisson H: The prognostic significance of tryptophanyl-tRNA synthetase in colorectal cancer. Cancer Epidemiol Biomarkers Prev 2009, 18:2949-2956.
43. Wallin U, Glimelius B, Jirström K, Darmanis S, Nong R Y, Pontén F, Johansson C, Påhlman L, Birgisson H: Growth differentiation factor 15: a prognostic marker for recurrence in colorectal cancer. Br J Cancer 2011,104:1619-1627.
44. Strömberg S, Agnarsdóttir M, Magnusson K, Rexhepaj E, Bolander A, Lundberg E, Asplund A, Ryan D, Rafferty M, Gallagher W M, Uhlen M, Bergqvist M, Ponten F: Selective expression of Syntaxin-7 protein in benign melanocytes and malignant melanoma. J Proteome Res 2009, 8:1639-1646.
45. Agnarsdóttir M, Sooman L, Bolander A, Strömberg S, Rexhepaj E, Bergqvist M, Ponten F, Gallagher W, Lennartsson J, Ekman S, Uhlen M, Hedstrand H: Sox10 expression in superficial spreading and nodular malignant melanomas. Melanoma Res 2010, 20:468-478.
46. Ryan D, Rafferty M, Hegarty S, O'Leary P, Faller W, Gremel G, Bergqvist M, Agnarsdottir M, Strömberg S, Kampf C, Pontén F, Millikan R C, Dervan P A, Gallagher W M: Topoisomerase I amplification in melanoma is associated with more advanced tumours and poor prognosis. Pigment Cell Melanoma Res 2010, 23:542-553.
47. Jaraj S J, Augsten M, Häggarth L, Wester K, Pontén F, Ostman A, Egevad L: GAD1 is a biomarker for benign and malignant prostatic tissue. Scand J Urol Nephrol 2011, 45:39-45.
48. Häggarth L, Hägglöf C, Jaraj S J, Wester K, Pontén F, Ostman A, Egevad L: Diagnostic biomarkers of prostate cancer. Scand J Urol Nephrol 2011, 45:60-67.
49. Kulasingam V, Pavlou M P, Diamandis E P: Integrating high-throughput technologies in the quest for effective biomarkers for ovarian cancer. Nat Rev Cancer 2010, 10:371-378.
50. Jemal A, Siegel R, Xu J, Ward E: Cancer statistics 2010. CA Cancer J Clin 2010, 60:277-300.
51. Poten F, Schwenk J M, Asplund A, Edgvist P H: The Human Protein Atlas as a proteomic resource for biomarker discovery. J Intern Med 2011,270:428-446.
52. Wu C C, Hsu C W, Chen C D, Yu C J, Chang K P, Tai D I, Liu H P, Su W H, Chang Y S, Yu J S: Candidate serological biomarkers for cancer identified from the secretomes of 23 cancer cell lines and the human protein atlas. Mol Cell Proteomics 2010, 9:1100-1117.
53. Griese M: Pulmonary surfactant in health and human lung diseases: State of the art. Eur Respir J 1999, 13:1455-1476.
54. Kuroki Y, Tsutahara S, Shijubo N, Takahashi H, Shiratori M, Hattori A, Honda Y, Abe S, Akino T: Elevated levels of lung surfactant protein a in sera from patients with idiopathic pulmonary fibrosis and pulmonary alveolar proteinosis. Am Rev Respir Dis 1993, 147:723-729.
55. Robin M, Dong P, Hermans C, Bernard A, Bersten A D, Doyle I R: Serum levels of CC16, SP-A and SP-B reflect tobacco-smoke exposure in asymptomatic subjects. Eur Respir J 2002, 20:1152-1161.
56. Greene K E, King T E, Kuroki Y, Bucher-Bartelson B, Hunninghake G W, Newman L S, Nagae H, Mason R J: Serum surfactant proteins-A and -D as biomarkers in idiopathic pulmonary fibrosis. Eur Respir J 2002, 19:439-446.
57. Goldberg D M: Proteases in the evaluation of pancreatic function and pancreatic disease. Clin Chim Acta 2000, 291:201-221.
58. Tomita T: Amylin in human pancreatic islets. Pathology 2003, 35:34-36.
59. Lonovics J, Devitt P, Watson L C, Rayford P L, Thompson J C: Pancreatic polypeptide. A review. Arch Surg 1981,116:1256-1264.
60. Lombardo D, Montalto G, Roudani S, Mas E, Laugier R, Sbarra V, Abouakil N: Is bile salt-dependent lipase concentration in serum of any help in pancreatic cancer diagnosis? Pancreas 1993, 8:581-588.
61. Millson C E, Charles K, Poon P, Made J, Mitchell C J: A prospective study of serum pancreatic elastase-1 in the diagnosis and assessment of acute pancreatitis. Scand J Gastroenterol 1998, 33:664-668.
62. Matsugi S, Hamada T, Shioi N, Tanaka T, Kumada T, Satomura S: Serum carboxypeptidase A activity as a biomarker for early-stage pancreatic carcinoma. Clin Chim Acta 2007, 378:147-153.
63. Fernstad R, Kylander C, Tsai L, Tyden G, Pousette A: Isoforms of procarboxypeptidase B, (pancreas-specific protein, PASP) in human serum, pancreatic tissue and juice. Scand J Clin Lab Invest Suppl 1993, 213:9-17.
64. Hayakawa T, Kondo T, Shibata T, Kigatawa M, Ono H, Sakai Y, Kiriyama S: Enzyme immunoassay for serum pancreatic lipase in the diagnosis of pancreatic disease. Gastroenterol Jpn 1989,24:556-560.
65. Adrian T E, Besterman H S, Mallinson C N, Pera A, Redshaw M R, Wood T P, Bloom S R: Plasma trypsin in chronic pancreatitis and pancreatic adenocarcinoma. Clin Chim Acta 1979, 97:205-212.
66. Killian C S, Emrich L J, Vargas F P, Yang N, Wang M C, Priore R L, Murphy G P, Chu T M: Relative reliability of five serially measured markers for prognosis of progression in prostate cancer. J Natl Cancer Inst 1986; 76:179-185.
67. Murphy G, Ragde H, Kenny G, Barren R 3rd, Erickson S, Tjoa B, Boynton A, Holmes E, Gilbaugh J, Douglas T: Comparison of prostate specific membrane antigen, and prostate specific antigen levels in prostatic cancer patients. Anticancer Res 1995, 15:1473-1479.
68. Recker F, Kwiatkowski M K, Piironen T, Pettersson K, Huber A, Lümmen G, Tscholl R: Human glandular kallikrein as a tool to improve discrimination of poorly differentiated and non-organ-confined prostate cancer compared with prostate-specific antigen. Urology 2000, 55:481-485.
69. Chen G, Gharib T G, Huang C C, Taylor J M, Misek D E, Kardia S L, Giordano T J, lannettoni M D, Orringer M B, Hanash S M, Beer D G: Discordant protein and mRNA expression in lung adenocarcinomas. Mol Cell Proteomics 2002, 1:304-313.
70. Pradet-Balade B, Boulme F, Beug H, Mullner E W, Garcia-Sanz J A: Translational control: bridging the gap between genomics and proteomics? Trends Biochem Sci 2001, 26:225-229.
71. Tian Q, Stepaniants S B, Mao M, Weng L, Feetham M C, Doyle M J, Yi E C, Dai H, Thorsson V, Eng J, Goodlett D, Berger J P, Gunter B, Linseley P S, Stoughton R B, Aebersold R, Collins S J, Hanlon W A, Hood L E: Integrated genomic and proteomic analyses of gene expression in mammalian cells. Mol Cell Proteomics 2004, 3:960-969.
72. GeneOntology Tools [http://geneontology.org/GO.tools.shtml].
73. Welsh J B, Sapinoso L M, Kern S G, Brown D A, Liu T, Bauskin A R, Ward R L, Hawkins N J, Quinn D I, Russell P J, Sutherland R L, Breit S N, Moskaluk C A, Frierson H F, Hampton G M: Large-scale delineation of secreted protein biomarkers overexpressed in cancer tissue and serum. Proc Natl Acad Sci USA 2003, 100:3410-3415.
74. Graddis T J, McMahan C J, Tamman J, Page K J, Trager J B: Prostatic acid phosphatase expression in human tissues. Int J Clin Exp Pathol 2011, 4:295-306.
75. Maitra A, Hruban R H: Pancreatic cancer. Annu Rev Pathol 2008, 3:157-188.
76. Magnani J L, Steplewski Z, Koprowski H, Ginsburg V: Identification of the gastrointestinal and pancreatic cancer-associated antigen detected by monoclonal antibody 19-9 in the sera of patients as a mucin. Cancer Res 1983, 43:5489-5492.
77. Marrelli D, Caruso S, Pedrazzani C, Neri A, Fernandes E, Marini M, Pinto E, Roviello F: CA19-9 serum levels in obstructive jaundice: clinical value in benign and malignant conditions. Am J Surg 2009, 198:333-339.
78. Nazli O, Bozdag A D, Tansug T, Kir R, Kaymak E: The diagnostic importance of CEA and CA 19-9 for the early diagnosis of pancreatic carcinoma. Hepatogastroenterology 2000, 47:1750-1752.
79. Tsavaris N, Kosmas C, Papadoniou N, Kopteridis P, Tsigritis K, Dokou A, Sarantonis J, Skopelitis H, Tzivras M, Gennatas K, Polyzos A, Papastratis G, Karatzas G, Papalambros A: CEA and CA-19.9 serum tumor markers as prognostic factors in patients with locally advanced (unresectable) or metastatic pancreatic adenocarcinoma: a retrospective analysis. J Chemother 2009, 21:673-680.
80. Gold D V, Modrak D E, Ying Z, Cardillo T M, Sharkey R M, Goldenberg D M: New MUC1 serum immunoassay differentiates pancreatic cancer from pancreatitis. J Clin Oncol 2006, 24:252-258.
81. Ringel J, Löhr M: The MUC gene family: their role in diagnosis and early detection of pancreatic cancer. Mol Cancer 2003, 2:9.
82. Rückert F, Pilarsky C, Grützmann R: Serum tumor markers in pancreatic cancer-recent discoveries. Cancers 2010, 2:1107-1124.
83. Robin X, Turck N, Hainard A, Lisacek F, Sanchez J C, Müller M: Bioinformatics for protein biomarker panel classification: what is needed to bring biomarker panels into in vitro diagnostics? Expert Rev Proteomics 2009, 6:675-689.
84. Tanase C P, Neagu M, Albulescu R, Hinescu M E: Advances in pancreatic cancer detection. Adv Clin Chem 2010, 51:145-180.
85. Yurkovetsky Z R, Linkov F Y, D E M, Lokshin A E: Multiple biomarker panels for early detection of ovarian cancer. Future Oncol 2006, 2:733-741.
86. Leong C T, Ng C Y, Ong C K, Ng C P, Ma Z S, Nguyen T H, Tay S K, Huynh H: Molecular cloning, characterization and isolation of novel spliced variants of the human ortholog of a rat estrogen-regulated membrane-associated protein, UO-44. Oncogene 2004, 23:5707-5718.
101. Siegel R, Naishadham D, Jemal A. Cancer statistics, 2012. CA: a cancer journal for clinicians. 2012; 62(1): 10-29.
102. Goonetilleke K S, Siriwardena A K. Systematic review of carbohydrate antigen (CA 19-9) as a biochemical marker in the diagnosis of pancreatic cancer. Eur J Surg Oncol. 2007; 33(3): 266-70.
103. Vincent A, Herman J, Schulick R, Hruban R H, Goggins M. Pancreatic cancer. Lancet. 2011; 378(9791): 607-20.
104. Conrad C, Lillemoe K D. Surgical palliation of pancreatic cancer. Cancer J. 2012; 18(6): 577-83.
105. Costello E, Greenhalf W, Neoptolemos J P. New biomarkers and targets in pancreatic cancer and their application to treatment. Nature reviews Gastroenterology & hepatology. 2012; 9(8): 435-44.
106. Bardeesy N, DePinho R A. Pancreatic cancer biology and genetics. Nature reviews. 2002; 2(12): 897-909.
107. Neoptolemos J P, Stocken D D, Friess H, Bassi C, Dunn J A, Hickey H, et al. A randomized trial of chemoradiotherapy and chemotherapy after resection of pancreatic cancer. The New England journal of medicine. 2004; 350(12): 1200-10.
108. Neoptolemos J P, Stocken D D, Tudur Smith C, Bassi C, Ghaneh P, Owen E, et al. Adjuvant 5-fluorouracil and folinic acid vs observation for pancreatic cancer: composite data from the ESPAC-1 and -3(v1) trials. British journal of cancer. 2009; 100(2): 246-50.
109. Hidalgo M. Pancreatic cancer. The New England journal of medicine. 2010; 362(17): 1605-17.
110. Ghaneh P, Costello E, Neoptolemos J P. Biology and management of pancreatic cancer. Gut. 2007; 56(8): 1134-52.
111. Chan A, Diamandis E P, Blasutig I M. Strategies for discovering novel pancreatic cancer biomarkers. Journal of proteomics. 2012.
112. Locker G Y, Hamilton S, Harris J, Jessup J M, Kemeny N, Macdonald J S, et al. ASCO 2006 update of recommendations for the use of tumor markers in gastrointestinal cancer. J Clin Oncol. 2006; 24(33): 5313-27.
113. Duffy M J, Sturgeon C, Lamerz R, Haglund C, Holubec V L, Klapdor R, et al. Tumor markers in pancreatic cancer: a European Group on Tumor Markers (EGTM) status report. Ann Oncol. 2010; 21(3): 441-7.
114. Makawita S, Smith C, Batruch I, Zheng Y, Ruckert F, Grutzmann R, et al. Integrated proteomic profiling of cell line conditioned media and pancreatic juice for the identification of pancreatic cancer biomarkers. Mol Cell Proteomics. 2011; 10(10): M111 008599.
115. Kosanam H, Makawita S, Judd B, Newman A, Diamandis E P. Mining the malignant ascites proteome for pancreatic cancer biomarkers. Proteomics. 2011; 11(23): 4551-8.
116. Kosanam H, Prassas I, Chrystoja C C, Soleas I, Chan A, Dimitromanolakis A, et al. LAMC2: A promising new pancreatic cancer biomarker identified by proteomic analysis of pancreatic adenocarcinoma tissues. Mol Cell Proteomics (submitted). 2012.
117. Prassas I, Chrystoja C C, Makawita S, Diamandis E P. Bioinformatic identification of proteins with tissue-specific expression for biomarker discovery. BMC medicine. 2012; 10: 39.
118. Makawita S, Dimitromanolakis A, Soosaipillai A, Soleas I, Chan A, Gallinger S, et al. Validation of four candidate pancreatic cancer serological biomarkers identifies multiple panels that significantly improve the performance of CA19.9. BMC Med (Submitted). 2012.
119. Chrystoja C C, Prassas I, Kosanam H, Chan A, Blasutig I M, Dimitromanolakis A, et al. CUB and zona pellucida-like domains 1 (CUZD1) is a novel serological biomarker for pancreatic adenocarcinoma. J Clin Oncol (Submitted). 2012.
120. Rennie D. Improving reports of studies of diagnostic tests: the STARD initiative. JAMA: the journal of the American Medical Association. 2003; 289(1): 89-90.
121. Robin X, Turck N, Hainard A, Tiberti N, Lisacek F, Sanchez J C, et al. pROC: an open-source package for R and S+ to analyze and compare ROC curves. BMC bioinformatics. 2011; 12: 77.
122. Pavlou M P, Diamandis E P, Blasutig I M. The Long Journey of Cancer Biomarkers from the Bench to the Clinic. Clinical chemistry. 2012.
123. Diamandis E P. Cancer biomarkers: can we turn recent failures into success? Journal of the National Cancer Institute. 2010; 102(19): 1462-7.
124. Campbell P J, Yachida S, Mudie U, Stephens P J, Pleasance E D, Stebbings L A, et al. The patterns and dynamics of genomic instability in metastatic pancreatic cancer. Nature. 2010; 467(7319): 1109-13.
125. Yachida S, Jones S, Bozic I, Antal T, Leary R, Fu B, et al. Distant metastasis occurs late during the genetic evolution of pancreatic cancer. Nature. 2010; 467(7319): 1114-7.
126. Faca V M, Song K S, Wang H, Zhang Q, Krasnoselsky A L, Newcomb L F, et al. A mouse to human search for plasma proteome changes associated with pancreatic tumor development. PLoS medicine. 2008; 5(6): e123.
127. Brand R E, Nolen B M, Zeh H J, Allen P J, Eloubeidi M A, Goldberg M, et al. Serum biomarker panels for the detection of pancreatic cancer. Clin Cancer Res. 2011; 17(4): 805-16.
130. Elisabeth R. DeLong, David M. DeLong and Daniel L. Clarke-Pearson (1988) “Comparing the areas under two or more correlated receiver operating characteristic curves: a nonparametric approach”. Biometrics 44, 837-845.

1. A method of evaluating a probability a subject has a cancer and/or diagnosing the subject with cancer, the method comprising:
a. measuring an amount of a biomarker selected from the group consisting of CUZD1 and/or LAMC2 and/or the group CUZD1, LAMC2, AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, GP73, DSG2, CEACAM7, CLCA1, GPA33, LEFTY1, ZG16, IRX5, LAMP3, MFAP4, SCGB1A1, SFTPC, TMEM100, NPY, PSCA, RLN1 and/or SLC45A3 in a test sample from a subject with cancer; wherein the cancer is pancreas cancer if CUZD1, LAMC2, AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, GP73 and/or DSG2 is selected; the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; or the cancer is prostate cancer if NPY, PSCA, RLN1 and/or SLC45A3 is selected;
b. comparing the measured amount to a control and detecting an increase in the amount of the biomarker compared to control; and
c. identifying the subject as having or having an increased probability of having the cancer when an increase in the biomarker compared to control is detected.

2. A method of monitoring cancer progression, the method comprising:
e. obtaining a test sample from the subject,
f. measuring an amount of biomarker according to the method of claim 1a.) in the test sample;
g. comparing the measured amount of biomarker in the test sample to the amount of biomarker in a base-line sample for the subject and/or a control; and
h. identifying a difference in the amount of the biomarker between the test sample and the base-line sample for the subject and/or the control;
wherein an increase in biomarker amount in the test sample compared to the base-line sample and/or the control is indicative of progression and a decrease in biomarker amount is indicative of lack of progression.

3. The method of claim 1 or 2, wherein the biomarkers comprise CUZD1 and/or LAMC2.

4. A method of monitoring pancreatic cancer progression, the method comprising:
e. obtaining a test sample from the subject,
f. measuring an amount of CUZD1 and/or LAMC2 in the test sample;
g. comparing the amount of CUZD1 and/or LAMC2 in the test sample to amount of CUZD1 and/or LAMC2 in a base-line sample for the subject and/or control; and
h. identifying a difference in the amount of the CUZD1 and/or LAMC2 between the test sample and the base-line sample and/or control;
wherein an increase in CUZD1 and/or LAMC2 in the test sample compared to the base-line sample is indicative of progression and a decrease in CUZD1 and/or LAMC2 is indicative of lack of progression.

5. A method of validating a candidate biomarker as a cancer biomarker comprising:
a. selecting a candidate biomarker from the group consisting of AQP8, CELA2B, CELA3B, CTRB1, CTRB2, CUZD1, GCG, IAPP, INS, LAMC2, KLK1, PNLIPRP1, PNLIPRP2, PPY, PRSS3, REG3G, SLC30A8, DSP, GP73, DSG2, CEACAM7, CLCA1, GPA33, LEFTY1, ZG16, IRX5, LAMP3, MFAP4, SCGB1A1, SFTPC, TMEM100, NPY, PSCA, RLN1 and/or SLC45A3 in a test sample from a subject with cancer, wherein the cancer is pancreas cancer if AQP8, CELA2B, CELA3B, CTRB1, CTRB2, GCG, IAPP, INS, KLK1, PNLIPRP1, PNLIPRP2, PPY, PRSS3, REG3G, SLC30A8, DSP, and/or GP73 is selected; the cancer is colon cancer if CEACAM7, CLCA1, GPA33, LEFTY1 and/or ZG16 is selected, the cancer is lung cancer if IRX5, LAMP3, MFAP4, SCGB1A1, SFTPC and/or TMEM100 is selected; or the cancer is prostate cancer if NPY, PSCA, RLN1 and/or SLC45A3 is selected;
b. measuring an amount of the selected candidate biomarker according to the method of claim 1 a.) in a plurality of samples from a plurality of subjects with cancer;
c. comparing the measured amount of the selected candidate biomarker in the plurality of test samples to a control;
d. identifying an increase in the amount of the selected candidate biomarker in the plurality of test samples as compared to the control; and
e. identifying a statistically significant increase in the amount of the selected candidate biomarker in the plurality of test samples as compared to the control;
wherein a statistically significant increased amount of the selected biomarker in the plurality of samples compared to the control is indicative the selected candidate biomarker is a cancer biomarker for the corresponding cancer.

6. The method of any one of claims 1 to 5, wherein the test sample is a biological fluid.

7. The method of claim 6 wherein the biological fluid is blood or a fraction thereof selected from serum and plasma.

8. The method of any one of claims 1-2 and 5, wherein the biomarkers is selected from CEACAM7, CLCA1, GPA33, LEFTY1 and/or ZG16.

9. The method of any one of claims 1-2 and 5, wherein the biomarker is selected from IRX5, LAMP3, MFAP4, SCGB1A1, SFTPC, SFTPD and TMEM100.

10. The method of any one of claims 1-2 and 5, wherein the biomarker is selected from AQP8, CELA2B, CELA3B, CPA1, CTRB1, CTRB2, CUZD1, GCG, IAPP, INS, KLK1, PNLIPRP1, PNLIPRP2, PPY, PRSS3, REG3G, SLC30A8, DSP, LAMC2, GP73 and/or DSG2.

11. The method of any one of claims 1-2 and 5, wherein the biomarker is selected from NPY, PSCA, RLN1 and SLC45A3.

12. The method of any one of claims 1-11, wherein the control is a cut-off for associated with a specificity and sensitivity and the specificity is selected to be at least 65%, at least 70%, at least 75%, at least 80%, at least 85% or at least 90%.

13. The method of any one of claims 1-12, wherein the sensitivity is selected to be at least 65%, at least 70%, at least 75%, at least 80%, at least 85% or at least 90%.

14. The method of any one of claims 1-13, wherein the amount of CUZD1 indicative the subject has pancreatic cancer or an increased probability of pancreatic cancer is greater than about 2 ng/ml, 2.2 ng/ml, 2.4 ng/ml, 2.6 ng/ml, 2.8 ng/ml, 3 ng/ml, 3.1 ng/ml, 3.2 ng/ml, 3.4 ng/ml, 3.6 ng/ml, 3.8 ng/ml, 4 ng/ml, 4.2 ng/ml, 4.4 ng/ml, 4.6 ng/ml, 4.8 ng/ml, 5 ng/ml.

15. The method of any one of claims 1-14, wherein the amount of LAMC2 indicative the subject has pancreatic cancer or an increased probability of pancreatic cancer is greater than about 100 ng/ml, 120 ng/ml, 140 ng/ml. 160 ng/ml, 170 ng/ml, 180 ng/ml, 200 ng/ml, 220 ng/ml, 240 ng/ml, 260 ng/ml, 280 ng/ml, 300 ng/ml, 320 ng/ml, 340 ng/ml, 360 ng/ml, 380 ng/ml or 400 ng/ml.

16. The method of any one of claims 1-15, further comprising measuring the amount of an additional biomarker in the sample.

17. The method of claim 16, wherein the additional biomarker is selected from CA19.9 CEA, CYFRA-21-1 NSE TPA, proGRP, SCC, CA125 and PSA.

18. The method of claim 16 wherein the additional biomarker is CA19.9

19. The method of claim 18 wherein the biomarker is CUZD1, LAMC2 and/or DSG2 and the additional biomarker is CA19.9.

20. The method of any one of claims 1 to 19, wherein the measuring comprises an antibody based immunoassay.

21. The method of claim 20, wherein the immunoassay is an ELISA.

22. Use of a biomarker selected from the group consisting of CUZD1 and/or LAMC2 and/or the group consisting of CEACAM7, CLCA1, GPA33, LEFTY1, ZG16, IRX5, LAMP3, MFAP4, SCGB1A1, SFTPC, TMEM100, AQP8, CELA2B, CELA3B, CTRB1, CTRB2, CUZD1, GCG, IAPP, INS, KLK1, PNLIPRP1, PNLIPRP2, PPY, PRSS3, REG3G, SLC30A8, KLK3, NPY, PSCA, RLN1, SLC45A3, DSP, LAMC2, GP73 and/or DSG2 for evaluating if a subject has cancer according to the method of any one of claims 1-4 and 6 to 21.

23. A method of validating a candidate biomarker as a soluble tissue specific cancer biomarker comprising:
a. selecting a candidate biomarker according to the method of claim 5a.);
b. measuring an amount of the selected candidate biomarker in a plurality of biological fluid test samples from a plurality of subjects afflicted by the cancer for the candidate marker and comparing to a control;
c. identifying an increase in the amount of the selected biomarker in the plurality of test samples as compared to the control; and;
d. identifying a statistically significant increase in the amount of the selected candidate biomarker in the plurality of biological fluid test samples as compared to the control;
wherein a statistically significant increased amount of the selected biomarker in the plurality of biological fluid test samples compared to the control is indicative the selected candidate biomarker is a soluble cancer biomarker for the corresponding cancer.

24. The method or use of any one of claims 6-23, wherein the biological fluid is selected from ascites, seminal plasma, peritoneal fluid, pancreatic juice and/or saliva.

25. The method or use of any one of claims 1 to 24, wherein 2, 3, 4, 5, 6, 7 or more biomarkers are measured.

26. The method or use of any one of claims 1 to 25 wherein the biomarkers comprise CUZD1, LAMC2 and CA19.9.

27. A kit comprising:
a. a biomarker specific reagent for a biomarker of the disclosure and optionally an additional biomarker; and
b. optionally one or more of
i. a kit standard;
ii. instructions for use and a vial housing the biomarker specific reagent and/or kit standard;
iii. reagents for qRT-PCR, including buffers, reverse transcription and amplification primers for the target genes and endogenous control genes, and control RNA from normal oral tissue;
iv. reagents for digital molecular barcoding technology, including for example buffers, hybridization solution, and/or one or more labeled probes;
v. collection tubes and/or assay plates for conducting one or more assays; and
vi. a sample collection vessel for example a vacutainer tube or other sterile tube for biological fluid.

28. The kit of claim 27, comprising two or more antibodies, optionally coupled to a solid surface.

29. The kit of claim 29, wherein the two or more antibodies comprise an antibody specific for CUDZ1 and an antibody specific for CA19.9.

30. The kit of claim 27-29, for use in the method or use of any one of claims 1 to 26.
